(19) United States
(12) Patent Application Publication
(10) Pub. No.: US 2020/0337973 A1
(43) Pub. Date: Oct. 29, 2020

(54) FLUORESCENT DIRECT DYE BEARING AN ALIPHATIC CHAIN AND A DISULFIDE/THIOL/PROTECTED-THIOL FUNCTION FOR DYEING KERATIN MATERIALS

(71) Applicant: L'OREAL, Paris (FR)

(72) Inventors: Christian BLAISE, AULNAY-SOUS-BOIS (FR); Virginie BURCKBUCHLER, AULNAY-SOUS-BOIS (FR)

(21) Appl. No.: 16/611,437

(22) PCT Filed: May 9, 2018
(86) PCT No.: PCT/EP2018/062038

§ 371 Date: Nov. 6, 2019

(30) Foreign Application Priority Data
May 10, 2017 (FR) 1754094

(51) Int. Cl. A61K 8/49; A61Q 5/06

(52) U.S. Cl. CPC A61K 8/4933; A61K 8/4926; A61Q 5/065; A61K 2800/30; A61K 2800/434; A61K 2800/10; A61K 2800/432

(57) ABSTRACT

The invention relates to a process for dyeing and/or lightening keratin materials, in particular keratin fibres, preferably human keratin fibres such as the hair, using at least one fluorescent direct dye bearing a disulfide, thiol or protected-thiol function and comprising at least one aliphatic chain of formula (I). The invention also relates to the use i) of at least one fluorescent direct dye bearing a disulfide, thiol or protected-thiol function (I) combined with ii) steam and/or a heating iron for straightening keratin fibres, for dyeing and lightening keratin materials, and to novel fluorescent direct dyes bearing a disulfide, thiol or protected-thiol function of formula (I), and also a cosmetic composition comprising same.

The process used and the use of the thiol, protected-thiol or disulfide fluorescent direct dyes of formula (I) combined with steam makes it possible especially to obtain long-lasting colouring with care valency on keratin materials.

The invention relates to a process for dyeing and/or lightening keratin materials, in particular keratin fibres, preferably human keratin fibres such as the hair, using at least one cationic, anionic, nonionic or zwitterionic direct fluorescent dye bearing a disulfide, thiol or protected-thiol function and comprising on at least one of the two chromophores at least one aliphatic chain of formula (I). The invention also relates to the use i) of at least one cationic, anionic, nonionic or zwitterionic fluorescent direct dye bearing a disulfide, thiol or protected-thiol function, comprising on at least one of the two chromophores at least one aliphatic chain of formula (I), combined with ii) steam and/or a heating iron for straightening keratin fibres, for the dyeing and/or lightening of keratin fibres, and of novel cationic, anionic, nonionic or zwitterionic fluorescent direct dyes bearing a disulfide, thiol or protected-thiol function, comprising on at least one of the two chromophores at least one aliphatic chain of formula (I), and also to a cosmetic composition comprising same.

It is known practice to dye keratin fibres by direct dyeing or semi-permanent dyeing. Direct dyeing or semi-permanent dyeing consists in introducing the colour via a coloured molecule which becomes adsorbed at the surface of the individual hair or which penetrates into the individual hair. Thus, the process conventionally used in direct dyeing consists in applying to keratin fibres direct dyes, which are coloured and colouring molecules that have affinity for the fibres, leaving the fibres in contact with the colouring molecules and then rinsing the fibres. Generally, this technique leads to chromatic colourings.

Scientific research has been conducted for several years to modify the colour of keratin materials, in particular keratin fibres, and in particular to mask white fibres, to modify the colour of the fibres permanently or temporarily, and to satisfy new desires and needs in terms of colours and durability.

Patent applications EP 1 647 580, WO 2005/097 051, EP 2 004 759, EP 2 075 289, WO 2007/110 541, WO 2007/110 540, WO 2007/110 539, WO 2007/110 538, WO 2007/110 537, WO 2007/110 536, WO 2007/110 535, WO 2007/110 534, WO 2007/110 533, WO 2007/110 532, WO 2007/110 531, EP 2 070 988, WO 2009/040 354 and WO 2009/034 059 disclose direct dyes bearing a disulfide, thiol or protected-thiol function, which may be grafted onto the hair using a reductive treatment. As it happens, the majority of reducing agents have a tendency to impair the integrity of keratin fibres, having the effect of making them brittle. In addition, when combined with dyes bearing a disulfide function, reducing agents generally generate unpleasant odours.

Moreover, the colourings obtained do not always give the keratin fibre a quality of softness and easy untangling, or the associated treatments are not always long-lasting.

The aim of the present invention is to provide novel systems for dyeing keratin materials, in particular keratin fibres, preferably human keratin fibres such as the hair, for obtaining colourings which are fast with respect to external agents, uniform and very strong, and/or which do not impair the cosmetic properties of keratin fibres, or even which give said materials care valency, and do so even without the use of a reducing agent and/or of a chemical oxidizing agent.

Another aim of the invention is to provide a dyeing system for obtaining visible colourings on naturally or artificially dark keratin materials, in particular keratin fibres, preferably human keratin fibres such as the hair, with lightening effects even in the absence of a chemical oxidizing agent, without degradation of the fibre, and whose colouring remains persistent with respect to external agents such as shampoos, while at the same time providing care to the fibres.

These aims are achieved with the present invention, the first subject of which is a process for dyeing keratin materials, in particular keratin fibres, especially human keratin fibres such as the hair, consisting in applying to said materials at least one cationic, anionic, nonionic or zwitterionic, preferably cationic, fluorescent direct dye bearing a disulfide function or a thiol or protected-thiol function, of formula (I):

A-(X)ₚ—Cₛₐₜ—S—U (I)

salts thereof with organic or mineral acids or bases, optical or geometric isomers thereof, tautomers thereof, and solvates thereof such as the hydrates;
in which formula (I):
U represents a radical chosen from: a) —S—C′ₛₐₜ—(X′)_{p′}-A′; and b) —Y;
A and A′, which may be identical or different, represent a cationic, anionic, nonionic or zwitterionic, preferably cationic, fluorescent chromophore, it being understood that at least one of the two chromophores bears at least one linear or branched, saturated or unsaturated C₁₀-C₃₀ aliphatic chain, preferably at least one of the two chromophores A and/or A′ bears at least one ammonium group bearing a linear or branched, saturated or unsaturated C₁₀-C₃₀ aliphatic chain;
Y represents i) a hydrogen atom; or ii) a thiol-function protecting group;
X and X′, which may be identical or different, represent a linear or branched, saturated or unsaturated divalent C₁-C₃₀ hydrocarbon-based chain, optionally interrupted and/or optionally terminated at one or both of its ends with one or more divalent groups or combinations thereof chosen from:
   —N(R)—, —N⁺(R)(R^{o})—, —O—, —C(O)—, —S(O)— and —S(O)₂—, with R and R^{o}, which may be identical or different, chosen from a hydrogen and a C₁-C₄ alkyl, hydroxyalkyl or aminoalkyl radical;
   an aromatic or non-aromatic, saturated or unsaturated, fused or non-fused (hetero)cyclic radical optionally comprising one or more identical or different, optionally substituted heteroatoms;
p and p′, which may be identical or different, are equal to 0 or 1, preferentially equal to 0;
Cₛₐₜ and C′ₛₐₜ, which may be identical or different, represent an optionally cyclic, optionally substituted, linear or branched C₁-C₁₈ alkylene chain;
it being understood that the electrical neutrality of the compounds of formula (I) may be ensured by one or more anionic or, respectively, cationic counterions depending on whether the dye is cationic or, respectively, anionic.

Another subject of the invention is the use, for dyeing keratin materials, in particular keratin fibres, preferably human keratin fibres such as the hair, of at least one fluorescent direct dye of formula (I) as defined previously, preferably combined with a treatment with steam and/or an iron for straightening the keratin fibres.

Another subject of the invention is a dye composition comprising at least one disulfide, thiol or protected-thiol fluorescent direct dye of formula (I) as defined previously.

Another subject of the invention relates to the compounds of formula (I) as defined previously.

The dyeing process of the invention in particular makes it possible to dye keratin materials, in particular keratin fibres, preferably human keratin fibres such as the hair, in a uniform, persistent manner, while providing care to said materials. Furthermore, the dyeing and care provided to said keratin materials are long-lasting.

In particular, the combination of the heat of the straightening iron or the steampod combined with the application of at least one fluorescent direct dye of formula (I) as defined previously, preferably bearing a disulfide function, dissolved in a hydrophilic or hydrophobic medium, gives the keratin materials, in particular keratin fibres, preferably human keratin fibres such as the hair a uniform colouring and persistent long-lasting care while at the same time respecting the integrity of the fibre (smooth feel, silky sheen appearance, facilitated disentangling when dry and in a wet medium, improved manageability). The colourings obtained are aesthetic, very strong, and very fast with respect to common attacking factors such as sunlight, perspiration, sebum and other treatments, especially hair treatments, for instance successive shampooing, while at the same time respecting said keratin materials.

In particular, the combination of the heat of the straightening iron or the steampod combined with the application of at least one fluorescent direct dye of formula (I) as defined previously, preferably bearing a disulfide function, dissolved in a hydrophilic or hydrophobic medium, gives sensitized hair a uniform colouring of the fibre and persistent long-lasting care while at the same time respecting the integrity of the fibre (smooth feel, silky sheen appearance, facilitated disentangling when dry and in a wet medium, improved manageability). The intensity obtained is particularly noteworthy. The same is true for the uniformity of the colour and of the care given to the keratin fibres, this being the case even after a heat treatment applied to the keratin fibres.

For the purposes of the present invention and unless otherwise indicated:
an “aliphatic chain” denotes a linear or branched, saturated or unsaturated C₁₀-C₃₀, preferably a C₁₂-C₂₀ and in particular a saturated, linear C₁₂-C₂₀ hydrocarbon-based radical;
a “cationic, anionic, nonionic or zwitterionic direct fluorescent dye bearing a disulfide function” is a direct dye comprising one or more cationic, anionic, nonionic or zwitterionic fluorescent chromophores, said dye absorbing light in the visible spectrum, preferably at a wavelength λ_{abs} particularly between 300 and 799 nm, and of re-emitting in the visible range at a longer wavelength λₑₘ than that absorbed, preferably between 400 and 800 nm;
The difference between the absorption and emission wavelengths, also known as the Stoke's shift, is between 1 nm and 100 nm. More preferentially, the fluorescent dyes of the invention are dyes that are capable of absorbing at a wavelength λ_{abs} of between 420 nm and 550 nm and of re-emitting in the visible range at a wavelength λₑₘ of between 470 and 600 nm;
a “fluorescent direct dye bearing a protected thiol function” is a cationic, anionic, nonionic or zwitterionic fluorescent direct dye comprising a chromophore, comprising a protected thiol function —SY in which Y is a protecting group for the thiol function known to those skilled in the art, for instance those described in the publications “Protective Groups in Organic Synthesis”, T. W. Greene, published by John Wiley & Sons, N Y, 1981, pages 193-217; “Protecting Groups”, P. Kocienski, Thieme, 3rd edition, 2005, chapter 5, and Ullmann's Encyclopedia, “Peptide Synthesis”, pages 4-5, 2005 Wiley-VCH Verlag GmbH & Co. KGaA, Weinheim 10.1002/14356007.a19 157;
a “fluorescent direct dye bearing a thiol function” is a cationic, anionic, nonionic or zwitterionic fluorescent direct dye comprising a chromophore, and comprising a thiol function —SY′ in which Y′ is i) a hydrogen atom; ii) an alkali metal; iii) an alkaline-earth metal; iv) an ammonium group: N⁺R^{α}R^{β}R^{γ}R^{δ} or a phosphonium group: P⁺R^{α}R^{β}R^{γ}R^{δ} with R^{α}, R^{β}, R^{γ} and R^{δ}, which may be identical or different, representing a hydrogen atom or a (C₁-C₄)alkyl group preferentially comprising a thiol function —SH;
a “chromophore” is a radical derived from a dye, i.e. a radical derived from a molecule that absorbs light in the visible radiation range that is visually and physiologically perceptible by man, i.e. at an absorption wavelength λ_{abs} inclusively between 300 and 799 nm; at least one of the two chromophores A and/or A′ is fluorescent, preferably at least one of the two chromophores A and/or A′ is capable of absorbing in the UV and visible radiation range at a wavelength λ_{abs} of between 350 and 600 nm, more particularly between 420 and 550 nm and capable of re-emitting in the visible range at an emission wavelength λₑₘ particularly between 400 and 800 nm, preferably between 470 and 600 nm;
a “chromophore” is said to be “cationic” if it comprises at least one cationic aryl or heteroaryl group as defined below, preferably an ammonium group included in or borne by a heteroaryl group, or an ammonium group borne by an aryl group;
a “chromophore” is said to be “anionic” if it comprises at least one aryl or heteroaryl group which comprises one or more anionic groups, chosen in particular from —SO₃⁻, M⁺; —C(O)O⁻, M⁺; —O—P(O)(OH)O⁻, M⁺ and —O—P(O)(O⁻M⁺)₂ with M⁺, which may be identical or different, present or absent, representing a cationic counterion which ensures the electrical neutrality of the molecule, such as an alkali metal such as Na⁺ and K+, an alkaline-earth metal, or ammonium;
a “chromophore” is said to be “nonionic” if it does not comprise any positively or negatively charged groups;
if the chromophore(s) A and/or A′ comprise one or more cationic groups and one or more anionic groups and if X and X′ do not contain —N+(R)(R^{o})—, then the dye will be said to be cationic if the total number of positive charges provided by the cationic group(s) is greater than the total number of negative charges provided by the anionic group(s),
if the chromophore(s) A and/or A′ comprise one or more anionic groups and one or more cationic groups and if X and X′ do not contain —N+(R)(R^{o})—, then the dye will be said to be anionic if the total number of negative charges provided by the anionic group(s) is greater than the total number of positive charges provided by the cationic group(s),
if the chromophore(s) A and/or A′ comprise one or more cationic groups and one or more anionic groups and if X and X′ do not contain —N+(R)(R^{o})—, then the dye will be said to be zwitterionic if the total number of positive charges provided by the cationic group(s) is equal to the total number of negative charges provided by the anionic group(s),
by way of example, if the dye of formula (I) comprises a chromophore A comprising a singly charged cationic group and if the chromophore A′ comprises a singly charged anionic group and if X and/or X′ comprise a group —N+(R)(R^{o})—, then the dye (I) will be said to be cationic;
the chromophores are said to be “different” when they differ in their chemical structure and may be chromophores derived from different families or from the same family on condition that they have different chemical structures: for example, the chromophores may be chosen from the family of styryl or naphthalimide dyes but differ in the chemical structure of the radicals constituting them or in the respective position of these radicals;
an “alkylene chain” represents a divalent C₁-C₁₈, particularly C₁-C₆ and more particularly C₁-C₂ chain when the chain is linear; optionally substituted with one or more identical or different groups chosen from hydroxyl, (C₁-C₂)alkoxy, (poly)hydroxy(C₂-C₄)alkoxy, (di)(C₁-C₂) (alkyl)amino, R^{a}—Z^{a}—C(Z^{b})— and R^{a}—Z^{a}—S(O)ₜ— with Z^{a}, Z^{b}, which may be identical or different, representing an oxygen atom, or a group NR^{a}′, R^{a} representing an alkali metal, a hydrogen atom or an alkyl group, or alternatively is absent, and R^{a}′ representing a hydrogen atom or an alkyl group and t is equal to 1 or 2;
   a “saturated or unsaturated, optionally substituted C₁-C₃₀ divalent hydrocarbon-based chain” represents a hydrocarbon-based chain, which is in particular a C₁-C₈ hydrocarbon-based chain, optionally comprising one or more conjugated or unconjugated π double bonds, the hydrocarbon-based chain being in particular saturated; said chain is optionally substituted with one or more identical or different groups chosen from hydroxyl, (C₁-C₂)alkoxy, (poly)hydroxy(C₂-C₄)alkoxy, (di)(C₁-C₂)(alkyl)amino, R^{a}—Z^{a}—C(Z^{b})— and R^{a}—Z^{a}—S(O)ₜ— with Z^{a}, Z^{b}, which may be identical or different, representing an oxygen atom, or a group NR^{a}′, R^{a} representing an alkali metal, a hydrogen atom or an alkyl group, or alternatively is absent, and R^{a}′ representing a hydrogen atom or an alkyl group and t is equal to 1 or 2;
the “aryl” or “heteroaryl” radicals or the aryl or heteroaryl part of a radical may be substituted with at least one substituent borne by a carbon atom, chosen from:
   a C₁-C₁₆ and preferably C₁-C₈ alkyl radical optionally substituted with one or more radicals chosen from hydroxyl, C₁-C₂ alkoxy, C₂-C₄ (poly)hydroxyalkoxy, C₁-C₆ acylamino such as acetylamino, amino substituted with two C₁-C₄ alkyl radicals, which may be identical or different, said alkyl radicals optionally bearing at least one hydroxyl group, or the two alkyl radicals possibly forming, with the nitrogen atom to which they are attached, a saturated or unsaturated, optionally substituted 5- to 7-membered and preferably 5- or 6-membered heterocycle optionally comprising another nitrogen or non-nitrogen heteroatom;
   a halogen atom;
   a hydroxyl group;
   a C₁-C₂ alkoxy radical;
   a (poly)hydroxy(C₂-C₄)alkoxy radical;
   an amino radical;
   a 5- or 6-membered heterocycloalkyl radical;
   an optionally cationic 5- or 6-membered heteroaryl radical, preferentially imidazolium, optionally substituted with a (C₁-C₄)alkyl radical, preferentially methyl;
   an amino radical substituted with one or two identical or different C₁-C₆ alkyl radicals, optionally bearing at least:
   i) a hydroxyl group,
   ii) an amino group optionally substituted with one or two optionally substituted C₁-C₃ alkyl radicals, said alkyl radicals possibly forming, with the nitrogen atom to which they are attached, a saturated or unsaturated, optionally substituted 5- to 7-membered heterocycle, optionally comprising at least one other nitrogen or non-nitrogen heteroatom,
   iii) a quaternary ammonium group —N+R′R″R′″, An⁻ for which R′, R″ and R′″, which may be identical or different, represent a hydrogen atom or a C₁-C₄ alkyl group and An⁻, which may be present or absent, represents the anionic counterion ensuring the electrical neutrality of the molecule,
   iv) or an optionally cationic 5- or 6-membered heteroaryl radical, preferentially imidazolium, optionally substituted with a (C₁-C₄)alkyl radical, preferentially methyl;
   an acylamino radical (—NR—C(O)R′) in which the radical R is a hydrogen atom, a C₁-C₄ alkyl radical optionally bearing at least one hydroxyl group and the radical R′ is a C₁-C₂ alkyl radical; a carbamoyl radical ((R)₂N—C(O)—) in which the radicals R, which may be identical or different, represent a hydrogen atom, a C₁-C₄ alkyl radical optionally bearing at least one hydroxyl group; an alkylsulfonylamino radical (R′—S(O)₂—N(R)—) in which the radical R represents a hydrogen atom, a C₁-C₄ alkyl radical optionally bearing at least one hydroxyl group and the radical R′ represents a C₁-C₄ alkyl radical or a phenyl radical; an aminosulfonyl radical ((R)₂N—S(O)₂—) in which the radicals R, which may be identical or different, represent a hydrogen atom or a C₁-C₄ alkyl radical optionally bearing at least one hydroxyl group,
   a carboxylic radical in acid or salified (preferably with an alkali metal or a substituted or unsubstituted ammonium) form;
   a cyano group;
   a nitro or nitroso group; or
   a polyhaloalkyl group, preferentially trifluoromethyl;
the cyclic or heterocyclic part of a non-aromatic radical may be substituted with at least one substituent chosen from the following groups:
   hydroxyl;
   C₁-C₄ alkoxy, C₂-C₄ (poly)hydroxyalkoxy;
   C₁-C₄ alkyl;
   alkylcarbonylamino (R—C(O)—N(R′)—) in which the radical R′ is a hydrogen atom or a C₁-C₄ alkyl radical optionally bearing at least one hydroxyl group and the radical R is a C₁-C₂ alkyl radical;
   alkylcarbonyloxy (R—C(O)—O—) in which the radical R is a C₁-C₄ alkyl radical;
   alkoxycarbonyl (R—C(O)—) in which the radical R is a C₁-C₄ alkoxy radical;
a cyclic or heterocyclic radical, or a non-aromatic part of an aryl or heteroaryl radical, may also be substituted with one or more oxo groups;
a hydrocarbon-based chain is unsaturated when it comprises one or more double bonds and/or one or more triple bonds, preferably one or more double bonds;
an “aryl” radical represents a monocyclic or fused or non-fused polycyclic carbon-based group comprising from 6 to 22 carbon atoms, at least one ring of which is aromatic; preferentially, the aryl radical is a phenyl, biphenyl, naphthyl, indenyl, anthracenyl or tetrahydronaphthyl;
a “heteroaryl radical” represents an optionally cationic, 5- to 22-membered, monocyclic or fused or non-fused polycyclic group, comprising from 1 to 6 heteroatoms chosen from nitrogen, oxygen, sulfur and selenium, at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl and xanthyl;
a “cyclic radical” is a non-aromatic, monocyclic or fused or non-fused polycyclic, cycloalkyl radical, containing from 5 to 22 carbon atoms, which may comprise one or more unsaturations;
a “heterocyclic radical” is a radical which may contain one or two unsaturations, but is a monocyclic or fused or non-fused polycyclic, 5- to 22-membered non-aromatic radical comprising from 1 to 6 heteroatoms chosen from nitrogen, oxygen, sulfur and selenium;
   a “heterocycloalkyl radical” is a saturated heterocyclic radical;
a “cationic heteroaryl radical” is a heteroaryl group as defined previously, which comprises an endocyclic or exocyclic cationic group,
   when the charge is endocyclic, it is included in the electron delocalization via the mesomeric effect; for example, it is a pyridinium, imidazolium or indolinium group:

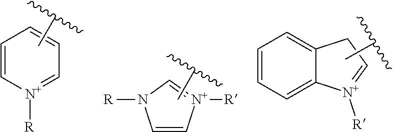

with R and R′ being a heteroaryl substituent as defined previously and preferably a (hydroxy)(C₁-C₃)alkyl group such as methyl or an aliphatic chain as defined previously, in particular C₁₄H₂₉, C₁₆H₃₃ or C₁₈H₃₇, preferentially n-C₁₄H₂₉, n-C₁₆H₃₃ or n-C₁₈H₃₇;
when the charge is exocyclic, it is not included in the electron delocalization via the mesomeric effect; for example, it is an ammonium or phosphonium R⁺ substituent, such as trimethylammonium, which is outside the heteroaryl, such as pyridyl, indolyl, imidazolyl or naphthalimidyl, in question:

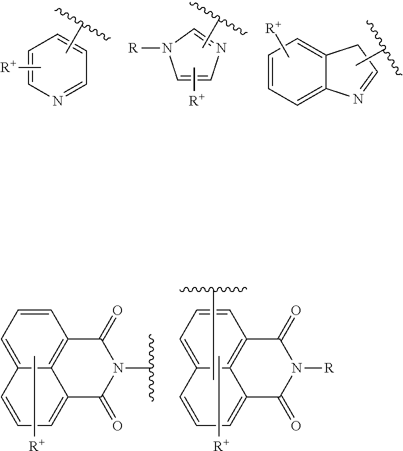

with R a heteroaryl substituent as defined previously and R⁺ an ammonium RₐR_{b}R_{c}N⁺—, phosphonium RₐR_{b}R_{c}P⁺— or ammonium RₐR_{b}R_{c}N⁺—(C₁-C₆)alkylamino group with Rₐ, R_{b} and R, which may be identical or different, representing a hydrogen atom or a (C₁-C₃₀)alkyl group such as methyl or an aliphatic chain as defined previously, in particular C₁₄H₂₉, C₁₆H₃₃ or C₁₈H₃₇, preferentially n-C₁₄H₂₉, n-C₁₆H₃₃ or n-C₁₈H₃₇;
the term “cationic aryl bearing an exocyclic charge” means an aryl ring whose cationic group is outside said ring; it is especially an R⁺ ammonium RₐR_{b}R_{c}N⁺—, phosphonium RₐR_{b}R_{c}P⁺— or ammonium RₐR_{b}R_{c}N⁺—(C₁-C₆)alkylamino substituent with Rₐ, R_{b} and R, which may be identical or different, representing a hydrogen atom or a (C₁-C₃₀)alkyl group such as methyl or an aliphatic chain as defined previously, in particular C₁₄H₂₉, C₁₆H₃₃ or C₁₈H₃₇, preferentially n-C₁₄H₂₉, n-C₁₆H₃₃ or n-C₁₈H₃₇; which is outside the aryl such as phenyl or naphthyl:

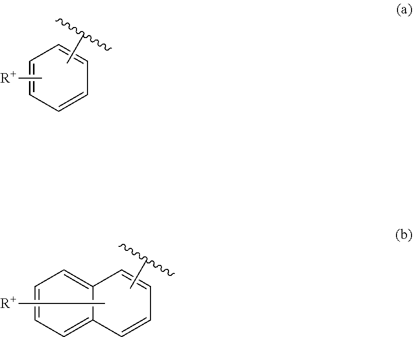

The cationic group R⁺ is borne directly by the aryl as represented in structures (a) and (b) above or is connected to the aryl via a linker arm LA as represented by structures (c1), (d1), (e1), (c2), (d2) and (e2) below,

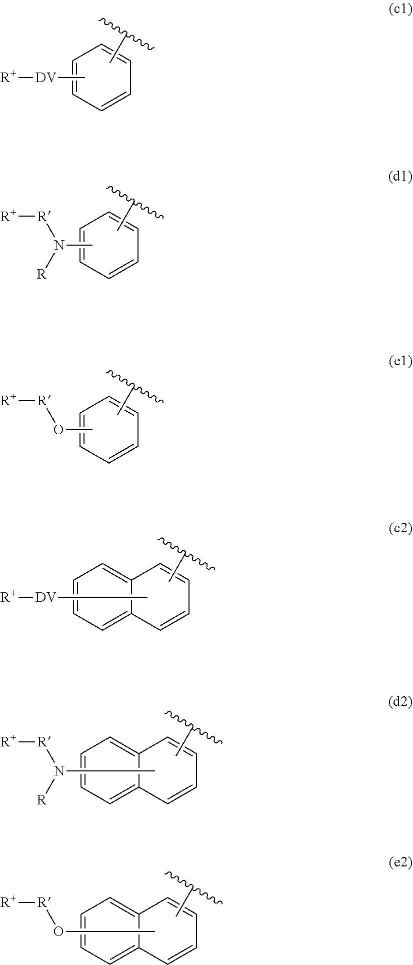

with LA representing:
   a linear or branched, saturated or unsaturated C₁-C₁₀, preferably saturated linear C₁-C₆ hydrocarbon-based divalent radical DV, one end of which is connected to R⁺, with R⁺ as defined previously;
   a divalent radical —N(R)—R′ in which R denotes a hydrogen atom or an optionally substituted alkyl radical such as methyl or ethyl, and R′ denotes a linear or branched, saturated or unsaturated C₁-C₁₀, preferably saturated linear C₁-C₆ hydrocarbon-based divalent radical which is connected to R⁺, with R⁺ as defined previously;
   a divalent radical —O—R′— in which R′ denotes a linear or branched, saturated or unsaturated C₁-C₁₀, preferably saturated linear C₁-C₆ hydrocarbon-based divalent radical which is connected to R⁺, with R⁺ as defined previously;
an “alkyl radical” is a linear or branched C₁-C₂₀ and preferably C₁-C₈ hydrocarbon-based radical;
an “alkenylene radical” is an alkyl radical as defined previously, containing from 1 to 4 conjugated or unconjugated double bonds —C═C—; the alkenylene group particularly contains 1 or 2 unsaturations;
the term “optionally substituted” attributed to the alkyl radical implies that said alkyl radical may be substituted with one or more radicals, which may be identical or different, chosen from the following radicals: i) hydroxyl, ii) C₁-C₄ alkoxy, iii) C₁-C₆ acylamino such as acetylamino, iv) amino optionally substituted with one or two identical or different C₁-C₄ alkyl radicals, said alkyl radicals possibly forming, with the nitrogen atom that bears them, a 5- to 7-membered heterocycle optionally comprising another nitrogen or non-nitrogen heteroatom; v) or a quaternary ammonium group —N⁺R′R″R′″, An⁻ for which R′, R″ and R′″, which may be identical or different, represent a hydrogen atom or a C₁-C₄ alkyl group, or alternatively —N⁺R′R″R′″ forms a heteroaryl such as imidazolium optionally substituted with a C₁-C₄ alkyl group, and An⁻, which may be present or absent, represents the anionic counterion which ensures the electrical neutrality of the molecule;
an “alkoxy radical” is an alkyl-oxy radical for which the alkyl radical is a linear or branched C₁-C₁₆ and preferentially C₁-C₈ hydrocarbon-based radical;
when the alkoxy group is optionally substituted, this implies that the alkyl group is optionally substituted as defined above;
an “ammonium group bearing a linear or branched, saturated or unsaturated C₁₀-C₃₀ aliphatic chain” is a group R_{α}R_{β}R_{χ}N⁺—, in which R_{α}, R_{β} and R_{χ}, which may be identical or different, represent a hydrogen atom or a (C₁-C₃₀)alkyl group such as methyl, it being understood that at least one of the groups R_{α}, R_{β} and R_{χ} denotes an aliphatic chain as defined previously, in particular C₁₄H₂₉, C₁₆H₃₃ or C₁₈H₃₇, preferentially n-C₁₄H₂₉, n-C₁₆H₃₃ or n-C₁₈H₃₇,
the “tone depth” is the unit known to hairstyling professionals, and published in the book “Science des traitements capillaires [Science of hair treatment]” by Charles ZVIAK 1988, published by Masson, pages 215 and 278; the tone depths range from 1 (black) to 10 (very light blond), one unit corresponding to one tone, the higher the figure, the lighter the shade;
a “dark” keratin fibre is a keratin fibre whose lightness L* measured in the CIE L*a*b* system is less than or equal to 45 and preferably less than or equal to 40, given that L*=0 is equivalent to black and L*=100 is equivalent to white;
“naturally or artificially dark hair” means hair whose tone depth is less than or equal to 6 (dark blond) and preferably less than or equal to 4 (chestnut-brown). Artificially dyed hair is hair whose colour has been modified by a dye treatment, for example dyeing with direct dyes or oxidation dyes;
the expression “at least one” means “one or more”.

1.1. Direct Fluorescent Dyes Bearing a Disulfide or Thiol Function of the Invention:

The fluorescent direct dye(s) bearing a disulfide, thiol or protected-thiol function used in the invention are of formula (I) as defined previously.

One particular mode of the invention relates to the fluorescent direct dyes bearing a disulfide function of formula (I) as defined previously, i.e. for which U represents the following radical a) —S—C′ₛₐₜ—(X′)_{p′}-A′, preferentially the disulfide dye(s) of the invention are symmetrical, i.e. comprise an axis of symmetry C₂, in other words, A=A′, Cₛₐₜ=C′ₛₐₜ, X=X′ and p=p′.

According to another particular mode of the invention, the fluorescent direct dyes of formula (I) are direct dyes bearing a thiol function as defined previously, i.e. U representing the radical b) Y.

Another particular embodiment of the invention relates to fluorescent dyes bearing a disulfide, thiol or protected-thiol function, for dyeing and/or lightening dark keratin fibres.

Preferentially, the fluorescent dyes of formula (I) bear a disulfide function i.e. U representing the radical —S—C′ₛₐₜ—(X′)_{p′}-A′. Even more preferentially, the fluorescent dyes of formula (I) bear a disulfide function and are symmetrical, i.e. A=A′, Cₛₐₜ=C′ₛₐₜ, X=X′ and p=p′.

1.1.1. Y:

According to a particular embodiment of the invention, the fluorescent direct dye of formula (I) is a thiol dye, i.e. Y represents i) a hydrogen atom.

In accordance with another particular embodiment of the invention, in the abovementioned formula (I), Y is a protecting group known to those skilled in the art, for instance those described in the publications “Protective Groups in Organic Synthesis”, T. W. Greene, published by John Wiley & Sons, N Y, 1981, pages 193-217; “Protecting Groups”, P. Kocienski, Thieme, 3rd edition, 2005, chapter 5, and Ullmann's Encyclopedia, “Peptide Synthesis”, pages 4-5, 2005 Wiley-VCH Verlag GmbH & Co. KGaA, Weinheim 10.1002/14356007.a19 157.

In particular, Y represents a thiol-function protecting group chosen from the following radicals:
(C₁-C₄)alkylcarbonyl;
(C₁-C₄)alkylthiocarbonyl;
(C₁-C₄)alkoxycarbonyl;
(C₁-C₄)alkoxythiocarbonyl;
(C₁-C₄)alkylthio-thiocarbonyl;
(di)(C₁-C₄)(alkyl)aminocarbonyl;
(di)(C₁-C₄)(alkyl)aminothiocarbonyl;
arylcarbonyl, for instance phenylcarbonyl;
aryloxycarbonyl;
aryl(C₁-C₄)alkoxycarbonyl;
(di)(C₁-C₄)(alkyl)aminocarbonyl, for instance dimethylaminocarbonyl;
(C₁-C₄)(alkyl)arylaminocarbonyl;
carboxyl;
SO₃⁻; M⁺ with M⁺, which may be present or absent, representing a cationic counterion which ensures the electrical neutrality of the molecule, such as an alkali metal such as sodium or potassium;
optionally substituted aryl such as phenyl, dibenzosuberyl or 1,3,5-cycloheptatrienyl;
optionally substituted heteroaryl; in particular including cationic or non-cationic heteroaryl comprising from 1 to 4 heteroatoms, chosen from i), ii) and iii):
   i) 5-, 6- or 7-membered monocyclic groups such as furanyl or furyl, pyrrolyl or pyrryl, thiophenyl or thienyl, pyrazolyl, oxazolyl, oxazolium, isoxazolyl, isoxazolium, thiazolyl, thiazolium, isothiazolyl, isothiazolium, 1,2,4-triazolyl, 1,2,4-triazolium, 1,2,3-triazolyl, 1,2,3-triazolium, 1,2,4-oxazolyl, 1,2,4-oxazolium, 1,2,4-thiadiazolyl, 1,2,4-thiadiazolium, pyrylium, thiopyridyl, pyridinium, pyrimidinyl, pyrimidinium, pyrazinyl, pyrazinium, pyridazinyl, pyridazinium, triazinyl, triazinium, tetrazinyl, tetrazinium, azepine, azepinium, oxazepinyl, oxazepinium, thiepinyl, thiepinium, imidazolyl, imidazolium;
   ii) 8- to 11-membered bicyclic groups such as indolyl, indolinium, benzimidazolyl, benzimidazolium, benzoxazolyl, benzoxazolium, dihydrobenzoxazolinyl, benzothiazolyl, benzothiazolium, pyridoimidazolyl, pyridoimidazolium, thienocycloheptadienyl, these monocyclic or bicyclic groups being optionally substituted with one or more groups such as (C₁-C₄)alkyl, for instance methyl, or polyhalo(C₁-C₄)alkyl, for instance trifluoromethyl;
   iii) the following tricyclic ABC radical:

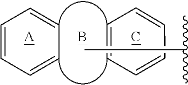

in which the two rings A and C optionally comprise a heteroatom, and ring B is a 5-, 6- or 7-membered ring, particularly a 6-membered ring, and contains at least one heteroatom, for instance piperidyl or pyranyl;
optionally cationic, optionally substituted heterocycloalkyl, the heterocycloalkyl group especially represents a saturated or partially saturated 5-, 6- or 7-membered monocyclic group comprising from 1 to 4 heteroatoms chosen from oxygen, sulfur and nitrogen, such as di/tetrahydrofuryl, di/tetrahydrothiophenyl, di/tetrahydropyrrolyl, di/tetrahydropyranyl, di/tetra/hexahydrothiopyranyl, dihydropyridyl, piperazinyl, piperidyl, tetramethylpiperidyl, morpholinyl, di/tetra/hexahydroazepinyl, di/tetrahydropyrimidinyl, these groups being optionally substituted with one or more groups such as (C₁-C₄) alkyl, oxo or thioxo; or the heterocycle represents the following group:

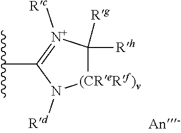

in which R′^{c}, R′^{d}, R′^{e}, R′^{f}, R′^{g} and R′^{h}, which may be identical or different, represent a hydrogen atom or a (C₁-C₄) alkyl group, or alternatively two groups R′^{g} with R′^{h}, and/or R′^{e} with R′^{f}, form an oxo or thioxo group, or alternatively R′^{g} with R′^{e} together form a cycloalkyl; and v represents an integer between 1 and 3 inclusive; preferentially, R′^{c} to R′^{h} represent a hydrogen atom; and An′″⁻, which may be present or absent, represents an anionic counterion which serves to ensure the electrical neutrality of the molecule;
—C(NR′^{c}R′^{d})═N⁺R′^{e}R′^{f}; An′″⁻ with R′^{c}, R′^{d}, R′^{e} and R′^{f}, which may be identical or different, representing a hydrogen atom or a (C₁-C₄)alkyl group; preferentially, R′^{c} to R′^{f} represent a hydrogen atom; and An′″⁻, which may be present or absent, represents an anionic counterion which serves to ensure the electrical neutrality of the molecule;
—C(NR′^{c}R′^{d})═NR′^{e}; with R′^{c}, R′^{d} and R′^{e} as defined previously;
optionally substituted (di)aryl(C₁-C₄)alkyl such as 9-anthracenylmethyl, phenylmethyl or diphenylmethyl optionally substituted with one or more groups especially chosen from (C₁-C₄) alkyl, (C₁-C₄) alkoxy such as methoxy, hydroxyl, alkylcarbonyl or (di)(C₁-C₄)(alkyl)amino such as dimethylamino;
optionally substituted (di)heteroaryl(C₁-C₄)alkyl, the heteroaryl group especially being a cationic or non-cationic, 5- or 6-membered monocyclic radical comprising from 1 to 4 heteroatoms chosen from nitrogen, oxygen and sulfur, such as pyrrolyl, furyl, thiophenyl, pyridyl, pyridyl N-oxide such as 4-pyridyl or 2-pyridyl-N-oxide, pyrylium, pyridinium or triazinyl groups, optionally substituted with one or more groups such as alkyl, particularly methyl; advantageously, the (di)heteroaryl(C₁-C₄)alkyl is (di)heteroarylmethyl or (di)heteroarylethyl;
CR¹R²R³ with R¹, R² and R³, which may be identical or different, representing a halogen atom or a group chosen from:
   (C₁-C₄)alkyl;
   (C₁-C₄)alkoxy;
   optionally substituted aryl such as phenyl optionally substituted with one or more groups, for instance (C₁-C₄)alkyl, (C₁-C₄)alkoxy or hydroxyl;
   optionally substituted heteroaryl such as thiophenyl, furyl, pyrrolyl, pyranyl or pyridyl, optionally substituted with a (C₁-C₄)alkyl group;
   P(Z¹)R′¹R′²R′³ with R′¹ and R′², which may be identical or different, representing a hydroxyl, (C₁-C₄)alkoxy or alkyl group, R′³ representing a hydroxyl or (C₁-C₄)alkoxy group, and Z¹ representing an oxygen or sulfur atom;
a sterically hindered ring; and
optionally substituted alkoxyalkyl, such as methoxymethyl (MOM), ethoxyethyl (EOE) and isobutoxymethyl.

According to one particular embodiment, the thiol-protected dyes of formula (I) comprise a group Y chosen from i) aromatic cationic 5- or 6-membered monocyclic heteroaryl comprising from 1 to 4 heteroatoms chosen from oxygen, sulfur and nitrogen, such as oxazolium, isoxazolium, thiazolium, isothiazolium, 1,2,4-triazolium, 1,2,3-triazolium, 1,2,4-oxazolium, 1,2,4-thiadiazolium, pyrylium, pyridinium, pyrimidinium, pyrazinyl, pyrazinium, pyridazinium, triazinium, tetrazinium, oxazepinium, thiepinyl, thiepinium, imidazolium; ii) cationic 8- to 11-membered bicyclic heteroaryl such as indolinium, benzimidazolium, benzoxazolium, benzothiazolium, these monocyclic or bicyclic heteroaryl groups optionally being substituted with one or more groups such as alkyl, for instance methyl, or polyhalo(C₁-C₄)alkyl such as trifluoromethyl; iii) or the following heterocyclic:

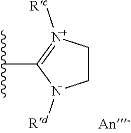

in which R′^{c} and R′^{d}, which may be identical or different, represent a hydrogen atom or a group (C₁-C₄)alkyl; preferentially R′^{c} to R′^{d} represent a group (C₁-C₄)alkyl such as methyl; and An′″⁻ represents a counterion.

In particular, Y represents a group chosen from oxazolium, isoxazolium, thiazolium, isothiazolium, 1,2,4-triazolium, 1,2,3-triazolium, 1,2,4-oxazolium, 1,2,4-thiadiazolium, pyrylium, pyridinium, pyrimidinium, pyrazinium, pyridazinium, triazinium and imidazolium, benzimidazolium, benzoxazolium, benzothiazolium, these groups being optionally substituted with one or more (C₁-C₄)alkyl groups, especially methyl.

In particular, Y represents a protecting group such as:
(C₁-C₄)alkylcarbonyl, for instance methylcarbonyl or ethylcarbonyl;
arylcarbonyl, for instance phenylcarbonyl;
(C₁-C₄)alkoxycarbonyl;
aryloxycarbonyl;
aryl(C₁-C₄)alkoxycarbonyl;
(di)(C₁-C₄)(alkyl)aminocarbonyl, for instance dimethylaminocarbonyl;
(C₁-C₄)(alkyl)arylaminocarbonyl;
optionally substituted aryl such as phenyl;
5- or 6-membered monocyclic heteroaryl such as imidazolyl or pyridyl;
cationic 5- or 6-membered monocyclic heteroaryl such as pyrylium, pyridinium, pyrimidinium, pyrazinium, pyridazinium, triazinium, imidazolium; these groups being optionally substituted with one or more identical or different (C₁-C₄)alkyl groups such as methyl;
cationic 8- to 11-membered bicyclic heteroaryl such as benzimidazolium or benzoxazolium; these groups being optionally substituted with one or more identical or different (C₁-C₄)alkyl groups such as methyl;
cationic heterocycle having the following formula:

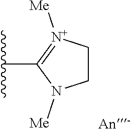 [SMILES: *C1=[N+](C)CCN1C]

—C(NH₂)═N⁺H₂; An′″⁻; with An′″⁻, which may be present or absent, representing an anionic counterion which serves to ensure the electrical neutrality of the molecule as defined previously;
—C(NH₂)═NH;
SO₃, M⁺ with M⁺, which may be present or absent, representing a cationic counterion which serves to ensure the electrical neutrality of the molecule, such as an alkali metal such as sodium or potassium.

1.1.2. Cₛₐₜ and C′ₛₐₜ:

As indicated previously, in formula (I), Cₛₐₜ and C′ₛₐₜ, independently of each other, represent a linear or branched, optionally substituted, optionally cyclic C₁-C₁₈ alkylene chain.

Substituents that may be mentioned include amino groups, (C₁-C₄)alkylamino groups, (C₁-C₄)dialkyl amino groups, or the group R^{a}—Z^{a}—C(Z^{b})— (in which Z^{a} and Z^{b}, which may be identical or different, represent an oxygen atom or a group NR^{a}′, and R^{a} represents an alkali metal, a hydrogen atom or a C₁-C₄ alkyl group and R^{a}′ represents a hydrogen atom or a C₁-C₄ alkyl group), said substituent(s) preferably being present on the carbon in the beta or gamma position relative to the sulfur atom(s) of the compounds of the dyes of formula (I).

Preferably, in the case of formula (I), Cₛₐₜ and C′ₛₐₜ represent a chain —(CH₂)ₖ— with k being an integer between 1 and 8 inclusive, more particularly between 1 and 4, such as 2. Preferably, Cₛₐₜ=C′ₛₐₜ.

1.1.3. X and X′:

In accordance with a particular embodiment of the invention, in the abovementioned formula (I), when p and p′ are equal to 1, X and X′, which may be identical or different, represent the following sequence: -(T)ₜ-(Z)_{z}-(T′)_{t′}—, said sequence being linked in formula (I) symmetrically as follows: —Cₛₐₜ-(T)ₜ-(Z)_{z}-(T′)_{t′}-A or —C′ₛₐₜ-(T)ₜ-(Z)_{z}-(T′)_{t′}-A′; in which:
T and T′, which may be identical or different, represent one or more radicals or combinations thereof chosen from: —O—; —N(R)—; —N⁺(R)(R^{o})—; —S(O)—; —S(O)₂—; —C(O)—; with R, R^{o}, which may be identical or different, representing a hydrogen atom, a C₁-C₄ alkyl radical, C₁-C₄ hydroxyalkyl radical or an aryl(C₁-C₄)alkyl radical; and a cationic or non-cationic, preferentially monocyclic heterocycloalkyl or heteroaryl radical, preferentially containing two heteroatoms (more preferentially two nitrogen atoms) and preferentially being 5- to 7-membered, more preferentially imidazolium; the indices t and t′, which may be identical or different, are equal to 0 or 1; preferably, t′=0;
Z represents:
   —(CH₂)ₘ— with m being an integer between 1 and 8 and preferably between 1 and 4;
   —(CH₂CH₂O)_{q}— or —(OCH₂CH₂)_{q}— in which q is an integer between 1 and 5 inclusive;
   an aryl, alkylaryl or arylalkyl radical in which the alkyl radical is C₁-C₄ and the aryl radical is preferably C₆, being optionally substituted with at least one group SO₃⁻ or SO₃H, with M⁺, which may be present or absent, representing a cationic counterion which serves to ensure the electrical neutrality of the molecule, such as an alkali metal or an ammonium group substituted with one or more identical or different, linear or branched C₁-C₈ alkyl radicals optionally bearing at least one hydroxyl;
z is 0 or 1.

Moreover, according to one particular embodiment of the invention, Z represents:

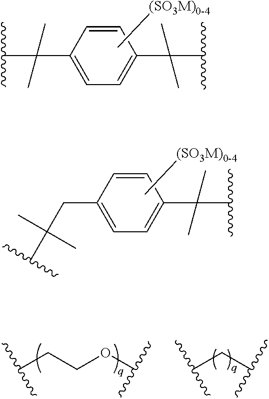

in which M, which may be identical or different, and which may be present or absent, represents a hydrogen atom or a cationic counterion which ensures the electrical neutrality of the molecule, such as an alkali metal or an ammonium group or an ammonium group substituted with one or more identical or different, linear or branched C₁-C₁₀ alkyl radicals optionally bearing at least one hydroxyl; 0-4 represents an integer between 0 and 4 inclusive, and q represents an integer between 1 and 6.

According to a particular embodiment of the invention, when p and p′ are equal to 1, X and X′ are identical.

According to a particularly preferred form of the invention, when p and p′ are equal to 1, X and X′ are identical and represent the following sequence: —N(R)—C(O)—(CH₂)ₘ—, said sequence being linked in formula (I) symmetrically as follows: —Cₛₐₜ—N(R)—C(O)—(CH₂)ₘ-A or —C′ₛₐₜ—N(R)—C(O)—(CH₂)ₘ-A′.

In accordance with a particular embodiment of the invention, in the abovementioned dyes of formula (I), p and p′ are equal to 0.

According to another particular embodiment of the invention, p and p′ are different. According to this embodiment, X or X′ preferably represents the sequence -(T)ₜ-(Z)_{z}-(T′)ₜ- as defined previously and preferably the sequence —N(R)—C(O)—(CH₂)ₘ— defined previously, said sequence being connected in formula (I) as follows: —Cₛₐₜ-(T)ₜ-(Z)_{z}-(T′)_{t′}-A or —C′ₛₐₜ-(T)ₜ-(Z)_{z}-(T′)_{t′}-A′.

1.1.4. A and A′:

According to a particular embodiment of the invention, the compounds of formula (I) are such that A and A′ independently denote a cationic, anionic, nonionic or zwitterionic fluorescent chromophore, at least one of the chromophores of which A and/or A′ bears a linear or branched C₁₀-C₃₀ aliphatic chain, said aliphatic chain preferably being linear, saturated or unsaturated, preferably saturated, and even more preferentially linear, saturated C₁₂-C₂₀.

According to a particular embodiment, at least one of the two chromophores A and/or A′ is cationic; preferably, the aliphatic chain constitutes one of the radicals of the cationic group of the chromophore(s) A and/or A′.

According to a particular embodiment, A and/or A′ does not bear an anionic radical and A and/or A′ bear(s) a trialkylammonium radical RₐR_{b}R_{c}N⁺—, An⁻, with Rₐ and R_{b}, which may be identical or different, representing a (C₁-C₃₀)alkyl group such as methyl and R_{c} representing a C₁₀-C₃₀, in particular C₁₄H₂₉, C₁₆H₃₃ or C₁₈H₃₇, preferentially n-C₁₄H₂₉, n-C₁₆H₃₃ or n-C₁₈H₃₇ aliphatic chain, and An⁻, which may be present or absent, representing an anionic counterion which serves to ensure the electrical neutrality of the molecule, such as a halide or an alkyl sulfate.

According to another particular embodiment of the invention, A and/or A′ does not bear an anionic radical and A and/or A′ denote a cationic aryl radical bearing an exocyclic charge, said charge preferably denoting a trialkylammonium radical RₐR_{b}R_{c}N⁺—, An⁻, with Rₐ and R_{b}, which may be identical or different, representing a (C₁-C₃₀)alkyl group such as methyl and R_{c} representing a C₁₀-C₃₀, in particular C₁₄H₂₉, C₁₆H₃₃ or C₁₈H₃₇, preferentially n-C₁₄H₂₉, n-C₁₆H₃₃ or n-C₁₈H₃₇ aliphatic chain, and An⁻, which may be present or absent, representing an anionic counterion which serves to ensure the electrical neutrality of the molecule, such as a halide or an alkyl sulfate.

According to a particular embodiment of the invention, the radicals A and A′ of formula (I) represent a cationic fluorescent chromophore, at least one of the two chromophores of which bears at least one ammonium group bearing a linear or branched, preferably linear, saturated or unsaturated, preferably saturated, C₁₀-C₃₀ aliphatic chain; more preferentially, the two fluorescent chromophores A and A′ each bear an ammonium group bearing a C₁₀-C₃₀, in particular C₁₄H₂₉, C₁₆H₃₃ or C₁₈H₃₇, preferentially n-C₁₄H₂₉, n-C₁₆H₃₃ or n-C₁₈H₃₇ aliphatic chain.

According to a preferred embodiment of the invention, the dyes (I) according to the invention are disulfides and comprise identical fluorescent chromophores A and A′.

More particularly, the fluorescent direct dyes of formula (I) according to the invention are disulfides and symmetrical, i.e. they contain a C₂ axis of symmetry, i.e. formula (I) is such that:

A-(X)ₚ—Cₛₐₜ—S—S—C′ₛₐₜ—(X′)_{p′}-A′

with A=A′, X=X′, p=p′, Cₛₐₜ=C′ₛₐₜ.

As cationic, anionic, nonionic or zwitterionic, preferably cationic, fluorescent chromophores that are useful in the present invention, mention may be made of those derived from the following dyes: acridines; acridones; anthranthrones; anthrapyrimidines; anthraquinones; azines; (poly)azos, hydrazono or hydrazones, in particular arylhydrazones; azomethines; benzanthrones; benzimidazoles; benzimidazolones; benzindoles; benzoxazoles; benzopyrans; benzothiazoles; benzoquinones; bisazines; bis-isoindolines; carboxanilides; coumarins; cyanines such as diazacarbocyanines, diazahemicyanines, tetraazacarbocyanines or (poly)methines such as dimethines of stilbene or styryl/hemicyanine type; diazines; diketopyrrolopyrroles; dioxazines; diphenylamines; diphenylmethanes; dithiazines; flavonoids such as flavanthrones and flavones; fluorindines; formazans; indamines; indanthrones; indigoids and pseudo-indigoids; indophenols; indoanilines; isoindolines; isoindolinones; isoviolanthrones; lactones; naphthalimides; naphthanilides; naphtholactams; naphthoquinones; nitro, in particular nitro(hetero)aromatics; oxadiazoles; oxazines; perilones; perinones; perylenes; phenazines; phenoxazine; phenothiazines; phthalocyanine; polyenes/carotenoids; porphyrins; pyranthrones; pyrazolanthrones; pyrazolones; pyrimidinoanthrones; pyronines; quinacridones; quinolines; quinophthalones; squaranes; tetrazoliums; thiazines, thioindigo; thiopyronines; triarylmethanes, or xanthenes. Preferentially, the chromophores are chosen from (poly)methine chromophores such as dimethines of styryl/hemicyanine type and naphthalimides.

In particular, as cationic, anionic, nonionic or zwitterionic, preferably cationic, fluorescent chromophores A and/or A′ that are useful in the present invention, mention may be made of radicals derived from the following dyes: acridines, acridones, benzanthrones, benzimidazoles, benzimidazolones, benzindoles, benzoxazoles, benzopyrans, benzothiazoles, coumarins, difluoro{2-[(2H-pyrrol-2-ylidene-kN)methyl]-1H-pyrrolato-kN}bores (BODIPY®), diketopyrrolopyrroles, fluorindines, (poly)methines such as dimethines (especially cyanines and styryls/hemicyanines), naphthalimides, naphthanilides, naphthylamine (such as dansyls), oxadiazoles, oxazines, perilones, perinones, perylenes, polyenes/carotenoids, squaranes, stilbenes and xanthenes.

Preferentially, the cationic, anionic, nonionic or zwitterionic, preferably cationic, fluorescent chromophores are chosen from (poly)methine chromophores such as dimethines of styryl/hemicyanine type and naphthalimides.

Mention may also be made of the fluorescent dyes A and/or A′ described in documents EP 1 133 975, WO 03/029 359, EP 860 636, WO 95/01772, WO 95/15144, EP 714 954 and those listed in the encyclopaedia “The chemistry of synthetic dye” by K. Venkataraman, 1952, Academic Press, vol. 1 to 7, in Kirk Othmer's encyclopaedia “Chemical Technology”, in the chapter “Dyes and Dye Intermediates”, 1993, Wiley and Sons, and in various chapters of “Ullmann's Encyclopedia of Industrial Chemistry” 7th edition, Wiley and Sons, and in The Handbook—A Guide to Fluorescent Probes and Labeling Technologies, 10th Ed Molecular Probes/Invitrogen—Oregon 2005 circulated on the Internet or in the preceding printed editions.

Among the cationic triarylmethane chromophores A and/or A′ that may be used according to the invention, mention may be made, besides those listed in the Colour Index, of the radicals derived from the following dyes: Basic Green 1; Basic Violet 3; Basic Violet 14; Basic Blue 7; Basic Blue 26.

Mention may also be made of the cationic chromophores derived from the dyes described in documents U.S. Pat. No. 5,888,252, EP 1 133 975, WO 03/029 359, EP 860 636, WO 95/01772, WO 95/15144 and EP 714 954. Mention may also be made of those listed in the encyclopaedia “The Chemistry of Synthetic Dyes” by K. Venkataraman, 1952, Academic Press, vol. 1 to 7, in the “Kirk-Othmer Encyclopedia of Chemical Technology”, in the chapter “Dyes and Dye Intermediates”, 1993, Wiley and Sons, and in various chapters of “Ullmann's Encyclopedia of Industrial Chemistry”, 7th edition, Wiley and Sons.

According to a preferred embodiment of the invention, the fluorescent direct dye(s) of formula (I) comprise on at least one of the two chromophores an ammonium group bearing a linear or branched, saturated or unsaturated C₁₀-C₃₀ aliphatic chain as defined previously, in particular of C₁₄H₂₉, C₁₆H₃₃ or C₁₈H₃₇, preferentially n-C₁₄H₂₉, n-C₁₆H₃₃ or n-C₁₈H₃₇,

According to an advantageous variant, A and/or A′ of formula (I) contain at least one cationic radical borne by, or included in, at least one of the chromophores. Preferably, said cationic radical is a quaternary ammonium; more preferentially, the cationic charge is exocyclic.

These cationic radicals are chosen, for example, from a cationic radical:
bearing a trialkylammonium exocyclic charge RₐR_{b}R_{c}N⁺—, with Rₐ and R_{b}, which may be identical or different, representing a (C₁-C₃)alkyl group such as methyl and R_{c} representing a C₁₀-C₃₀, in particular C₁₄H₂₉, C₁₆H₃₃ or C₁₈H₃₇, preferentially n-C₁₄H₂₉, n-C₁₆H₃₃ or n-C₁₈H₃₇ aliphatic chain which is in particular on a naphthalimidyl chromophore, and
bearing an endocyclic charge, such as the following cationic heteroaryl groups: acridinium, benzimidazolium, benzobistriazolium, benzopyrazolium, benzopyridazinium, benzoquinolium, benzothiazolium, benzotriazolium, benzoxazolium, bipyridinium, bis-tetrazolium, dihydrothiazolium, imidazopyridinium, imidazolium, indolium, isoquinolium, naphthoimidazolium, naphthoxazolium, naphthopyrazolium, oxadiazolium, oxazolium, oxazolopyridinium, oxonium, phenazinium, phenoxazolium, pyrazinium, pyrazolium, pyrazoyltriazolium, pyridinium, pyridinoimidazolium, pyrrolium, pyrylium, quinolium, tetrazolium, thiadiazolium, thiazolium, thiazolopyridinium, thiazoylimidazolium, thiopyrylium, triazolium or xanthylium.

According to one preferred variant of the invention, the cationic fluorescent chromophore(s) A and/or A′ comprise at least one quaternary ammonium radical bearing an aliphatic chain such as those derived from the polymethine dyes of formulae (II) and (II′) below:

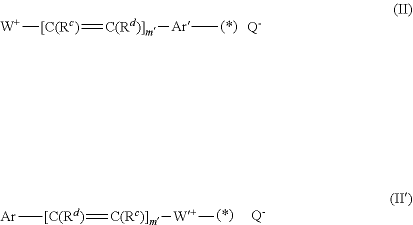

formula (II) or (II′) in which:
W⁺ represents a cationic heterocyclic or heteroaryl group, particularly comprising a quaternary ammonium bearing an aliphatic chain, said heterocyclic or heteroaryl group being optionally substituted with one or more (C₁-C₈)alkyl groups optionally substituted especially with one or more hydroxyl groups;
W′⁺ represents a cationic heterocyclic or heteroaryl divalent radical in particular comprising a quaternary ammonium, said heterocyclic or heteroaryl group being optionally substituted with one or more (C₁-C₈)alkyl groups optionally substituted especially with one or more hydroxyl groups; Ar represents an aryl group such as phenyl or naphthyl bearing an exocyclic ammonium charge, with an aliphatic chain, optionally substituted preferentially with i) one or more halogen atoms such as chlorine or fluorine; ii) one or more (C₁-C₈)alkyl groups, preferably of C₁-C₄ such as methyl; iii) one or more hydroxyl groups; iv) one or more (C₁-C₈)alkoxy groups such as methoxy; v) one or more hydroxy(C₁-C₈)alkyl groups such as hydroxyethyl, vi) one or more amino or (di)(C₁-C₈)alkylamino groups, preferably with the C₁-C₄ alkyl part optionally substituted with one or more hydroxyl groups, such as (di)hydroxyethylamino, vii) with one or more acylamino groups; viii) one or more heterocycloalkyl groups such as piperazinyl, piperidyl or 5- or 6-membered heteroaryl such as pyrrolidinyl, pyridyl and imidazolinyl;
Ar′ is a divalent aryl radical such as phenyl or naphthyl, optionally substituted preferentially with i) one or more halogen atoms such as chlorine or fluorine; ii) one or more (C₁-C₈)alkyl and preferably (C₁-C₄)alkyl groups such as methyl; iii) one or more hydroxyl groups; iv) one or more (C₁-C₈)alkoxy groups such as methoxy; v) one or more hydroxy(C₁-C₈)alkyl groups such as hydroxyethyl, vi) one or more amino or (di)(C₁-C₈)alkylamino groups, preferably with the C₁-C₄ alkyl part optionally substituted with one or more hydroxyls, such as (di)hydroxyethylamino, vii) with one or more acylamino groups; viii) one or more heterocycloalkyl groups such as piperazinyl, piperidyl or 5- or 6-membered heteroaryl such as pyrrolidinyl, pyridyl and imidazolinyl;
m′ represents an integer between 1 and 4 inclusive; in particular, m is 1 or 2; more preferentially 1;
R^{c} and R^{d}, which may be identical or different, represent a hydrogen atom or an optionally substituted (C₁-C₈)alkyl group, preferentially of C₁-C₄, or alternatively R^{C} contiguous with W⁺ or W′⁺ and/or R^{d} contiguous with Ar or Ar′ form, with the atoms that bear them, a (hetero)cycloalkyl; in particular, R^{c} is contiguous with W⁺ or W′⁺ and forms a (hetero)cycloalkyl such as cyclohexyl; preferably, R^{c} and R^{d} denote a hydrogen atom;
Q⁻, which may be present or absent, is an anionic counterion which serves to ensure the electrical neutrality of the molecule;
(*) represents the part of the chromophore linked to the rest of formula (I);
it being understood that at least one of the groups W⁺, W′⁺, Ar and Ar′ bears at least one ammonium group bearing a linear or branched, saturated or unsaturated C₁₀-C₃₀ aliphatic chain; preferably, the ammonium group(s) are R¹R²R³N⁺—, An⁻ with R¹ representing a linear or branched (C₁₀-C₃₀)alkyl group or a linear or branched (C₁₀-C₃₀)alkenyl group, in particular C₁₄H₂₉, C₁₆H₃₃ or C₁₈H₃₇, preferentially n-C₁₄H₂₉, n-C₁₆H₃₃ or n-C₁₈H₃₇; and R² and R³, which may be identical or different, represent a hydrogen atom or a linear or branched (C₁-C₃₀)alkyl or linear or branched (C₂-C₃)alkenyl group, preferably a (C₁-C₆)alkyl group, and An⁻, which may be present or absent, is an anionic counterion which serves to ensure the electrical neutrality of the molecule, such as a halide or an alkyl sulfate.

According to another variant, the disulfide, thiol or protected-thiol fluorescent dye of the invention is a cationic fluorescent dye such that, in formula (I): A and/or A′ represent a naphthalimidyl radical bearing an exocyclic cationic charge of formula (III) or (III′) as defined below.

According to another variant, the disulfide, thiol or protected-thiol fluorescent dye of the invention is a cationic fluorescent dye comprising at least one ammonium radical bearing an aliphatic chain and such that, in formula (I), preferably with p and p′ equal to 0 and A and/or A′ representing a naphthalimidyl radical bearing an exocyclic cationic charge of formula (III) or (III′):

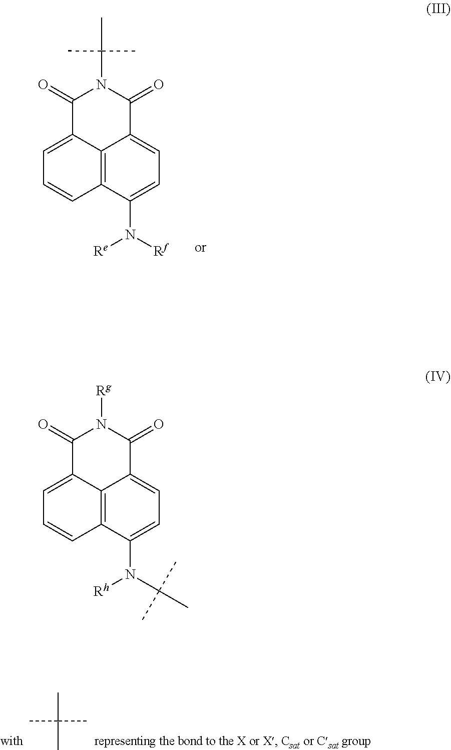

in which formulae (III) and (III′) R^{e}, R^{f}, R^{g} and R^{h}, which may be identical or different, represent a hydrogen atom or a C₁-C₆ alkyl group, of which at least one of the groups R^{e} or R^{f} of the naphthalimidyl (III), or alternatively R^{g} or R^{h} of the naphthalimidyl (IV) is substituted with a trialkylammonium group RₐR_{b}R_{c}N⁺—, An⁻, with Rₐ and R_{b}, which may be identical or different, representing a (C₁-C₃₀)alkyl group such as methyl and R_{c} representing a C₁₀-C₃₀, in particular C₁₄H₂₉, C₁₆H₃₃ or C₁₈H₃₇, preferentially n-C₁₄H₂₉, n-C₁₆H₃₃ or n-C₁₈H₃₇ aliphatic chain, and An⁻, which may be present or absent, representing an anionic counterion which ensures the electrical neutrality of the molecule, such as a halide or an alkyl sulfate.

According to a particular embodiment, the disulfide, thiol or protected-thiol fluorescent dye of the invention is a cationic fluorescent dye such that, in formula (I), A and/or A′ represent a naphthalimidyl radical bearing an exocyclic cationic charge of formula (III) or (III′) as defined previously, p is equal to 0, Cₛₐₜ represents —(CH₂)ₖ— with k being an integer between 1 and 8 inclusive, more particularly between 1 and 4, such as 2.

According to a preferred embodiment, the fluorescent dye of the invention is a disulfide cationic fluorescent dye such that, in formula (I), A=A′ and A represents a naphthalimidyl radical bearing an exocyclic cationic charge of formula (III) as defined previously, p is equal to 0, Cₛₐₜ represents —(CH₂)ₖ— with k being an integer between 1 and 8 inclusive, more particularly between 1 and 4, such as 2. According to this embodiment, A preferably represents a naphthalimidyl radical bearing an exocyclic cationic charge of formula (III) such that R^{e} represents a hydrogen atom and R^{f} represents a C₁-C₆ alkyl group substituted with a trialkylammonium group RₐR_{b}R_{c}N⁺—, An⁻, with Rₐ and R_{b}, which may be identical or different, representing a (C₁-C₃₀)alkyl group such as methyl and R_{c} representing a C₁₀-C₃₀, in particular C₁₄H₂₉, C₁₆H₃₃ or C₁₈H₃₇, preferentially n-C₁₄H₂₉, n-C₁₆H₃₃ or n-C₁₈H₃₇ aliphatic chain, and An⁻ representing an anionic counterion which ensures the electrical neutrality of the molecule, such as a halide or an alkyl sulfate.

According to one embodiment of the invention, the dye(s) of formula (I) are such that the cationic fluorescent chromophore(s) A and/or A′ comprise at least one quaternary ammonium radical bearing an aliphatic chain such as those derived from the polymethine dyes of formula (II′) defined previously, preferably in the para position on Ar relative to the styryl function —C(R^{o})═C(R^{d})—. According to this embodiment, preferably, the dyes of formula (I) are such that p=1, and X represents —N(R)—CO—(CH₂)ₘ— with R and m as defined previously, Cₛₐₜ=—(CH₂)ₖ— with k being an integer between 1 and 8 inclusive, more particularly between 1 and 4, such as 2.

Preferably, the dyes of formula (I) are such that W′⁺ is an imidazolium, pyridinium, benzimidazolium, pyrazolium, benzothiazolium or quinolinium optionally substituted with one or more identical or different C₁-C₄ alkyl radicals, in particular a pyridinium.

According to a particularly preferred embodiment of the invention, A and/or A′ represent the chromophore (II′) as defined previously with m′=1, Ar representing a phenyl group which is substituted, preferably para to the styryl group —C(R^{d})═C(R^{o})—, with a trialkylammonium group RₐR_{b}R_{c}N⁺—, An⁻, with Rₐ and R_{b}, which may be identical or different, representing a (C₁-C₃₀)alkyl group such as methyl and R_{c} representing a C₁₀-C₃₀, in particular C₁₄H₂₉, C₁₆H₃₃ or C₁₈H₃₇, preferentially n-C₁₄H₂₉, n-C₁₆H₃₃ or n-C₁₈H₃₇ aliphatic chain, and An⁻, which may be present or absent, representing an anionic counterion which ensures the electrical neutrality of the molecule, such as a halide or an alkyl sulfate; and W′⁺ representing an imidazolium or pyridinium, preferentially ortho- or para-pyridinium, more preferentially para-pyridinium, group.

As examples of dyes of formula (I) of the invention, mention may be made of the disulfide dyes chosen from formulae (IV) to (VIII) and the thiol or protected-thiol dyes chosen from formulae (IV′) to (VIII′) below:

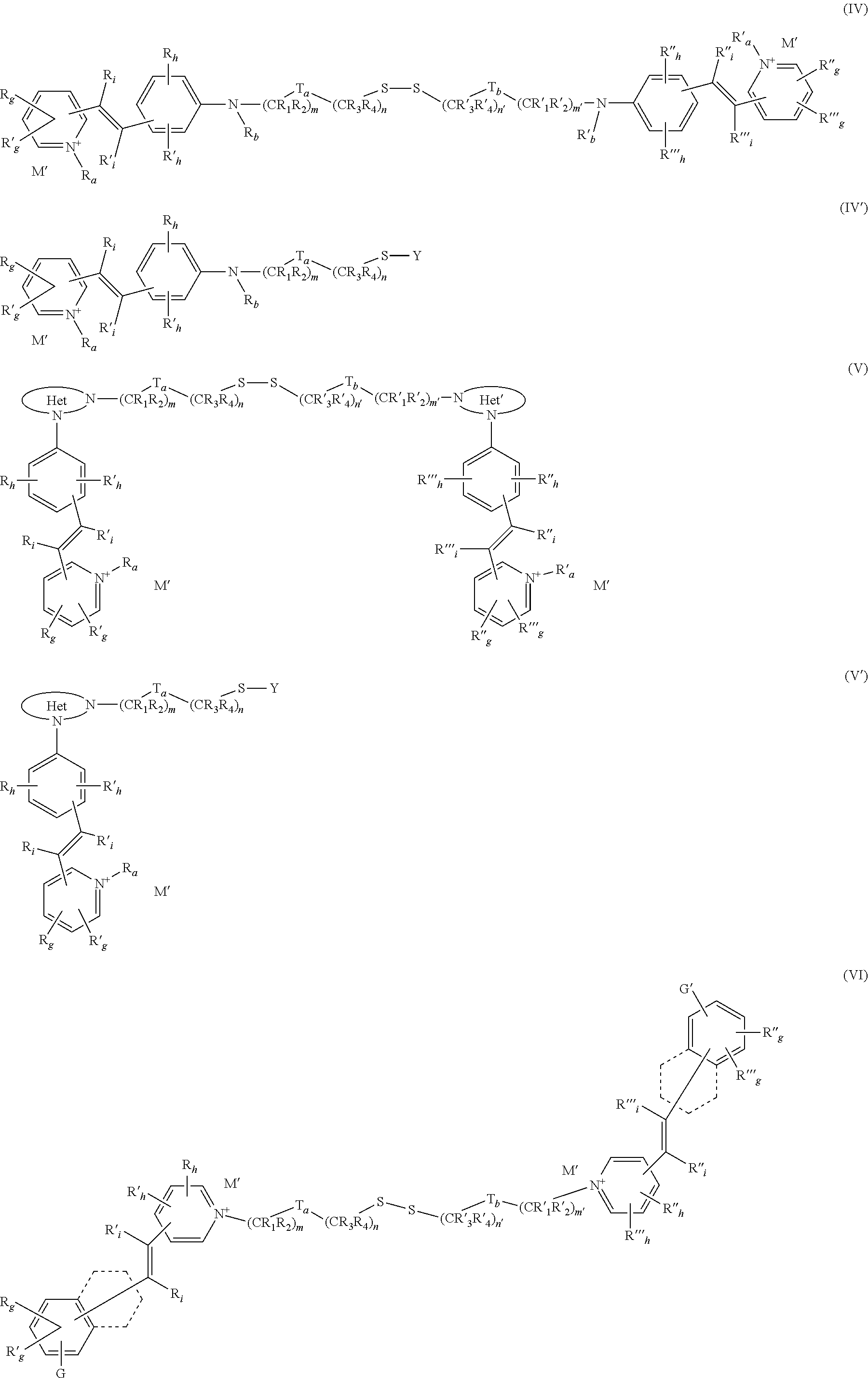

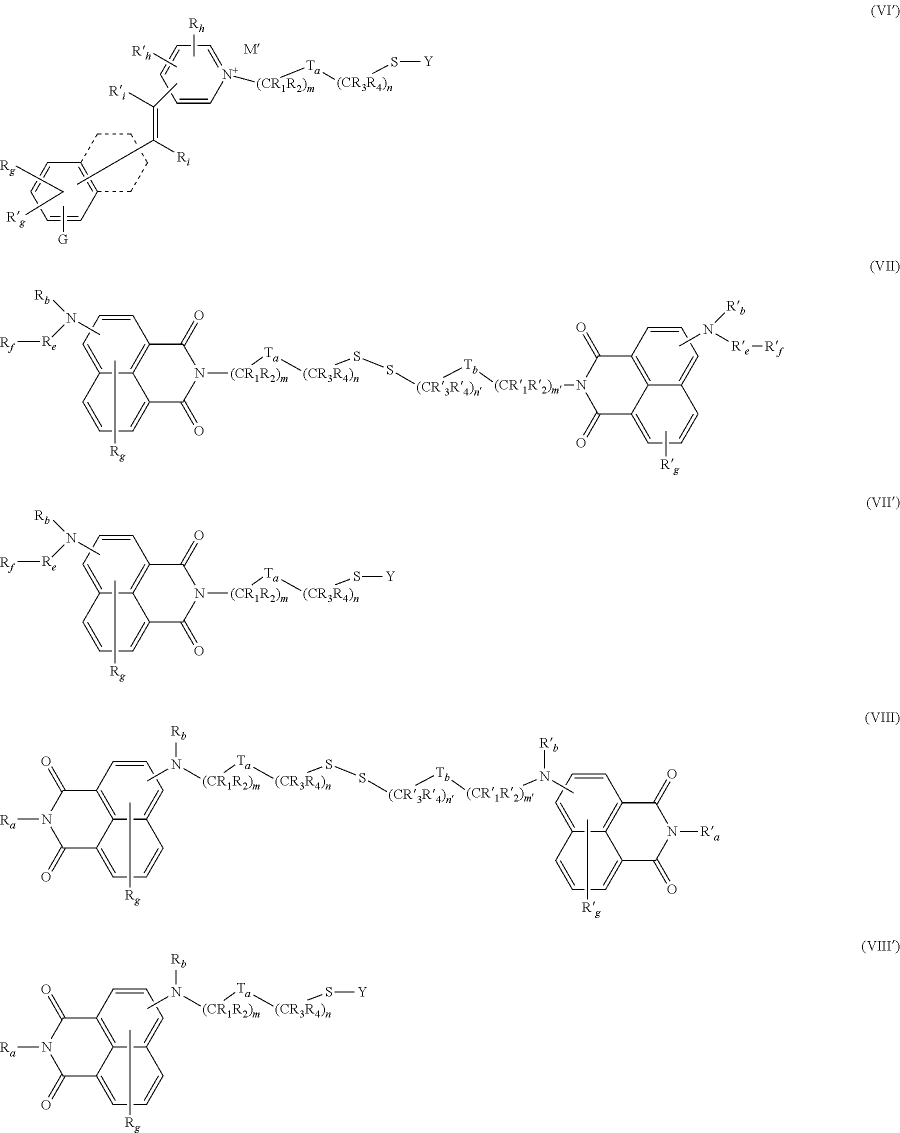

formulae (IV) to (VIII) and (IV′) to (VII′), the organic or mineral acid salts, optical isomers and geometrical isomers, and the solvates such as hydrates, in which formulae:
G and G′, which may be identical or different, represent i) a group —NR_{c}R_{d}, —NR′_{c}R′_{d}, ii) C₁-C₆ alkoxy which is optionally substituted, preferentially unsubstituted; or iii) a group R¹R²R³N⁺—, An⁻ with R¹ representing a linear or branched (C₁₀-C₃₀)alkyl, in particular C₁₄H₂₉, C₁₆H₃₃ or C₁₈H₃₇, preferentially n-C₁₄H₂₉, n-C₁₆H₃₃ or n-C₁₈H₃₇ group; and R² and R³, which may be identical or different, represent a linear or branched (C₁ C₃₀) alkyl group, preferably a (C₁-C₆)alkyl group such as methyl, and An⁻, which may be present or absent, represents an organic or mineral anionic counterion such as a halide or an alkyl sulfate;
Rₐ and R′ₐ, which may be identical or different, represent an aryl(C₁-C₄)alkyl group or a C₁-C₆ alkyl group optionally substituted with a hydroxyl or amino, C₁-C₄ alkylamino or C₁-C₄ dialkylamino group, said alkyl radicals possibly forming, with the nitrogen atom that bears them, a 5- to 7-membered heterocycle, optionally comprising another nitrogen or non-nitrogen heteroatom; preferentially, Rₐ and R′ₐ represent a C₁-C₃ alkyl group optionally substituted with a hydroxyl group, or a benzyl group; preferably, Rₐ and R′ₐ are identical;
R_{b} and R′_{b}, which may be identical or different, represent a hydrogen atom, an aryl(C₁-C₄)alkyl group or a C₁-C₆ alkyl group that is optionally substituted; preferentially, R_{b} and R′_{b} represent a hydrogen atom or a C₁-C₃ alkyl or benzyl group; preferably, R_{b} and R′_{b} are identical and represent a hydrogen atom;
R_{c}, R′_{c}, R_{d} and R′_{d}, which may be identical or different, represent a hydrogen atom, an aryl(C₁-C₄)alkyl or C₁-C₆ alkoxy group or a C₁-C₆ alkyl group that is optionally substituted; R, R′, R_{d} and R′_{d} preferentially represent a hydrogen atom, a hydroxyl, C₁-C₃ alkoxy, amino or C₁-C₃ (di)alkylamino group, or a C₁-C₃ alkyl group that is optionally substituted with i) a hydroxyl group, ii) amino, iii) C₁-C₃ (di)alkylamino, or iv) quaternary ammonium R¹R²R³N⁺—, An⁻ as defined previously;
or alternatively two adjacent radicals R, and R_{d}, R′_{c} and R′_{d} borne by the same nitrogen atom together form a heterocyclic or heteroaryl group; preferentially, the heterocycle or heteroaryl is monocyclic and 5- to 7-membered; more preferentially, the groups are chosen from imidazolyl and pyrrolidinyl;
Rₑ and R′ₑ, which may be identical or different, represent a linear or branched, optionally unsaturated, divalent C₁-C₆ alkylenyl hydrocarbon-based chain; preferably, Rₑ and R′ₑ, which are identical, represent a linear divalent C₁-C₆ alkylenyl hydrocarbon-based chain such as a —(CH₂)₃— chain;
R_{f} and R′_{f}, which may be identical or different, represent a quaternary ammonium group R¹R²R³N⁺—, An⁻ as defined previously;
R_{g}, R′_{g}, R″_{g}, R′″_{g}, Rₕ, R′ₕ, R″ₕ, and R′″ₕ, which may be identical or different, represent a hydrogen atom, a halogen atom, an amino, C₁-C₄ alkylamino, C₁-C₄ dialkylamino, cyano, carboxyl, hydroxyl or trifluoromethyl group, an acylamino, C₁-C₄ alkoxy, (poly)hydroxy(C₂-C₄)alkoxy, alkylcarbonyloxy, alkoxycarbonyl or alkylcarbonylamino radical, an acylamino, carbamoyl or alkylsulfonylamino radical, an aminosulfonyl radical, or a C₁-C₁₆ alkyl radical optionally substituted with a group chosen from C₁-C₁₂ alkoxy, hydroxyl, cyano, carboxyl, amino, C₁-C₄ alkylamino and C₁-C₄ dialkylamino, or alternatively the two alkyl radicals borne by the nitrogen atom of the amino group form a 5- to 7-membered heterocycle optionally comprising another heteroatom identical to or different from that of the nitrogen atom; preferentially, R_{g}, R′_{g}, R″_{g}, R′″_{g}, Rₕ, R′ₕ, R″ₕ, and R′″ₕ represent a hydrogen or halogen atom or a C₁-C₃ alkyl group; more preferentially, R_{g}, R′_{g}, Rₕ and R′ represent a hydrogen atom;
or alternatively two groups R_{g} and R′_{g}; R″_{g} and R′″_{g}; Rₕ and R′ₕ; R″ₕ and R′″ₕ borne by two adjacent carbon atoms together form a benzo or indeno ring, a fused heterocycloalkyl or fused heteroaryl group; the benzo, indeno, heterocycloalkyl or heteroaryl ring being optionally substituted with a halogen atom, an amino, C₁-C₄ alkylamino, C₁-C₄ dialkylamino, nitro, cyano, carboxyl, hydroxyl or trifluoromethyl group, an acylamino, C₁-C₄ alkoxy, (poly)hydroxy(C₂-C₄)alkoxy, alkylcarbonyloxy, alkoxycarbonyl or alkylcarbonylamino radical, an acylamino, carbamoyl or alkylsulfonylamino radical, an aminosulfonyl radical, or a C₁-C₁₆ alkyl radical optionally substituted with: a group chosen from C₁-C₁₂ alkoxy, hydroxyl, cyano, carboxyl, amino, C₁-C₄ alkylamino, C₁-C₄ dialkylamino, or alternatively the two alkyl radicals borne by the nitrogen atom of the amino group form a 5- to 7-membered heterocycle optionally comprising another heteroatom identical to or different from that of the nitrogen atom; preferentially, R_{g} and R′_{g}; R″_{g} and R′″_{g} together form a benzo group;
or alternatively when G represents —NR_{c}R_{d} and G′ represents —NR′_{c}R′_{d}, two groups R_{c} and R′_{g}; R′_{c} and R″_{g}; R_{d} and R_{g}; R′_{d} and R′″_{g} together form a saturated heteroaryl or heterocycle, optionally substituted with one or more groups C₁-C₆ alkyl, preferentially a 5- to 7-membered heterocycle containing one or two heteroatoms chosen from nitrogen and oxygen; more preferentially the heterocycle is chosen from morpholinyl, piperazinyl, piperidyl and pyrrolidinyl groups;
Rᵢ, R′ᵢ, R″ᵢ, and R′″ᵢ, which may be identical or different, represent a hydrogen atom or a C₁-C₄ alkyl group; preferably, Rᵢ and R′ᵢ represent a hydrogen atom; R₁, R₂, R₃, R₄, R′₁, R′₂, R′₃, and R′₄, which may be identical or different, represent a hydrogen atom or a C₁-C₄ alkyl, C₁-C₁₂ alkoxy, hydroxyl, cyano, carboxyl, amino, C₁-C₄ alkylamino or C₁-C₄ dialkylamino group, said alkyl radicals possibly forming with the nitrogen atom that bears them a 5- to 7-membered heterocycle optionally comprising another nitrogen or non-nitrogen heteroatom; preferentially, R₁, R₂, R₃, R₄, R′₁, R′₂, R′₃, and R′₄ are hydrogen atoms or an amino group; more preferentially, R₁, R₂, R₃, R₄, R′₁, R′₂, R′₃, and R′₄ represent a hydrogen atom; Tₐ, T_{b}, which may be identical or different, represent i) either a σ covalent bond, ii) or one or more radicals or combinations thereof chosen from —SO₂—, —O—, —S—, —N(R)—, —N⁺(R)(R^{o})— and —CO—, with R and R^{o}, which may be identical or different, representing a hydrogen atom, a C₁-C₄ alkyl radical or a C₁-C₄ hydroxyalkyl; or an aryl(C₁-C₄)alkyl radical; preferentially, Tₐ is identical to T_{b} and they represent a σ covalent bond or a group chosen from —N(R)—, —C(O)—N(R)—, —N(R)—C(O)—, —O—C(O)—, —C(O)—O— and —N⁺(R)(R^{o})—, with R and R^{o}, which may be identical or different, representing a hydrogen atom or a C₁-C₄ alkyl group; more preferentially, Tₐ and T_{b} represent a σ bond or —C(O)—N(R)— or —N(R)—C(O)—; iii) or a cationic or non-cationic, preferentially monocyclic heterocycloalkyl or heteroaryl radical, which are preferentially identical, preferentially containing two heteroatoms (more preferentially two nitrogen atoms) and preferentially being 5- to 7-membered, such as imidazolium; preferably, Tₐ represents a group —N(R)—C(O)—, with R representing a hydrogen atom or a C₁-C₄ alkyl group and more preferentially with R denoting a hydrogen atom or Tₐ represents a σ covalent bond;

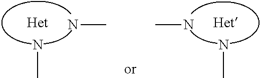

which may be identical or different, represent a heterocyclic group, which is optionally substituted, preferably with a quaternary ammonium group R¹R²R³N⁺—, An⁻ as defined previously; preferentially, the heterocycles are identical, monocyclic, saturated, and comprise in total two nitrogen atoms and are 5- to 8-membered;

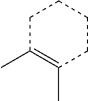

represents an aryl or heteroaryl group fused to the imidazolium or phenyl ring; or alternatively is absent from the imidazolium or phenyl ring; preferentially, when the ring is present, the ring is a benzo;
m, m′, n and n′, which may be identical or different, represent an integer between 0 and 6 inclusive with m+n and m′+n′, which may be identical or different, representing an integer between 1 and 10 inclusive; preferentially, m+n=m′+n′=an integer between 2 and 8 inclusive; more preferentially, m+n=an integer between 1 and 8 inclusive and more preferentially between 1 and 6; even more preferentially, m+n=5 or m+n=2; Y is as defined previously; in particular, Y represents a hydrogen atom or a protecting group such as:
   (C₁-C₄)alkylcarbonyl, for instance methylcarbonyl or ethylcarbonyl;
   arylcarbonyl, for instance phenylcarbonyl;
   (C₁-C₄)alkoxycarbonyl;
   aryloxycarbonyl;
   aryl(C₁-C₄)alkoxycarbonyl;
   (di)(C₁-C₄)(alkyl)aminocarbonyl, for instance dimethylaminocarbonyl;
   (C₁-C₄)(alkyl)arylaminocarbonyl;
   optionally substituted aryl such as phenyl;
   5- or 6-membered monocyclic heteroaryl such as imidazolyl or pyridyl;
   cationic 5- or 6-membered monocyclic heteroaryl such as pyrylium, pyridinium, pyrimidinium, pyrazinium, pyridazinium, triazinium, imidazolium; these groups being optionally substituted with one or more identical or different (C₁-C₄)alkyl groups such as methyl;
   cationic 8- to 11-membered bicyclic heteroaryl such as benzimidazolium or benzoxazolium; these groups being optionally substituted with one or more identical or different (C₁-C₄)alkyl groups such as methyl;
   cationic heterocycle having the following formula:

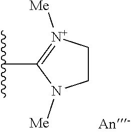 [SMILES: *C1=[N+](C)CCN1C]

—C(NH₂)═N⁺H₂; An′″⁻; with An′″⁻ being an anionic counterion as defined previously;
—C(NH₂)═NH;
SO₃⁻, M⁺ with M⁺ representing an alkali metal such as sodium or potassium; and
M′ represents an anionic counterion, derived from a salt of an organic or mineral acid, or from an organic or mineral base that ensures the electrical neutrality of the molecule;
it being understood that at least one of the groups G, G′, Rₐ, R′, R, R′, R_{d}, R′_{d}, R_{f} or R′_{f} bears at least one group R¹R²R³N⁺—, An⁻ as defined previously.

In particular, the dyes of formula (I) are chosen from dyes with a naphthalimidyl disulfide, thiol or protected-thiol chromophore, chosen from formulae (VII), (VII′), (VIII) and (VIII′) as defined previously, more particularly from the dyes (VII) and (VII′) and even more particularly (VII).

According to another form of the invention, the dyes of formula (I) are chosen from disulfide, thiol or protected-thiol dyes bearing a styryl chromophore, chosen from formulae (VI) and (VI′) as defined previously, and more particularly from the dyes (VI).

According to a preferred mode of the invention, the dyes of formula (I) are chosen from disulfide, thiol or protected-thiol dyes chosen from formulae (IX) to (X′) below:

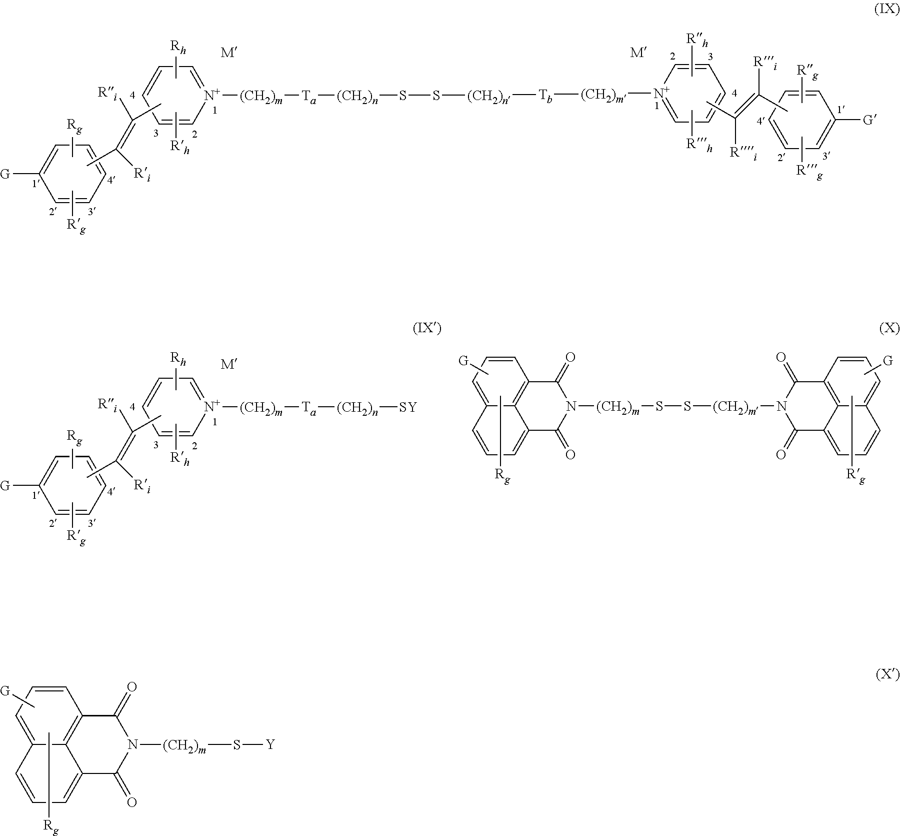

the organic or mineral acid salts, optical isomers and geometrical isomers thereof, and the solvates such as hydrates;
in which formulae (IX) to (X′):
G and G′, which may be identical or different, represent i) a (di)(C₁-C₆)alkylamine group with at least one of the alkyl groups substituted with an ammonium group R¹R²R³N⁺—, An⁻ as defined previously or ii) an ammonium group R¹R²R³N⁺—, An⁻ as defined previously; preferably, G and G′ are identical;
R_{g}, R′_{g}, R″_{g}, R′″_{g}, Rₕ, R′ₕ, R″ₕ and R′″ₕ, which may be identical or different, represent a hydrogen or halogen atom, an amino, (di)(C₁-C₄)alkylamino, cyano, carboxyl, hydroxyl, trifluoromethyl, acylamino, C₁-C₄ alkoxy, C₂-C₄ (poly)hydroxyalkoxy, (C₁-C₄)alkylcarbonyloxy, (C₁-C₄)alkoxycarbonyl, (C₁-C₄)alkylcarbonylamino, acylamino, carbamoyl or (C₁-C₄)alkylsulfonylamino group, an aminosulfonyl radical or a (C₁-C₁₆)alkyl radical optionally substituted with a group chosen from (C₁-C₁₂)alkoxy, hydroxyl, cyano, carboxyl, amino and (di)(C₁-C₄)alkylamino, or alternatively the two alkyl radicals borne by the nitrogen atom of the amino group form a 5- to 7-membered heterocycle optionally comprising another nitrogen or non-nitrogen heteroatom; in particular, R_{g}, R′_{g}, R″_{g}, R′″_{g}, Rₕ, R′ₕ, R″ₕ and R′″ₕ represent a hydrogen atom or a (C₁-C₄)alkyl group; preferably, R_{g}, R′_{g}, R″_{g}, R′″_{g}, Rₕ, R′ₕ, R″ₕ and R′″ₕ represent a hydrogen atom;
R′ᵢ, R″ᵢ, R″ᵢ and R′″ᵢ, which may be identical or different, represent a hydrogen atom or a (C₁-C₄)alkyl group; in particular R′ᵢ, R″ᵢ, R′″ᵢ and R″″ᵢ represent a hydrogen atom;
Tₐ and T_{b}, which may be identical or different, represent i) either a σ covalent bond, ii) or one or more radicals or combinations thereof chosen from —O—, —N(R)— and —C(O)—, with R representing a hydrogen atom or a C₁-C₄ alkyl radical; preferentially Tₐ is identical to T_{b} and represents a σ covalent bond or a group chosen from —N(R)—, —C(O)—N(R)— and —N(R)—C(O)—, with R representing a hydrogen atom or a C₁-C₄ alkyl group; more preferentially, Tₐ and T_{b}, which are identical, represent —C(O)—N(R)— or —N(R)—C(O)— with R representing a hydrogen atom or a C₁-C₄ alkyl group and more preferentially with R representing a hydrogen atom;
m, m′, n and n′, which may be identical or different, represent an integer between 0 and 6 inclusive, with m+n and m′+n′, which may be identical or different, representing an integer between 1 and 10 inclusive; preferentially, m+n=m′+n′=and m+n an integer between 1 and 8 inclusive, more preferentially between 1 and 6; even more preferentially, m+n=5 or m=2;
Y is as defined previously.

According to a particular mode of the invention, the dyes of the invention belong to formula (IX) or (IX′) and bear an ethylene group linking the pyridinium part to the phenyl ortho or para to the pyridinium, i.e. at 2-4′, 4-2′ or 4-4′, preferably in the 4-4′ para position.

Advantageously, the dyes of formulae (IX) and (IX′) bear their ethylene group para to the phenyl bearing the group G or G′, i.e. in position 1′-4′.

According to a particular mode of the invention, the dyes of the invention belong to formula (IXa) or (IX′a) below:

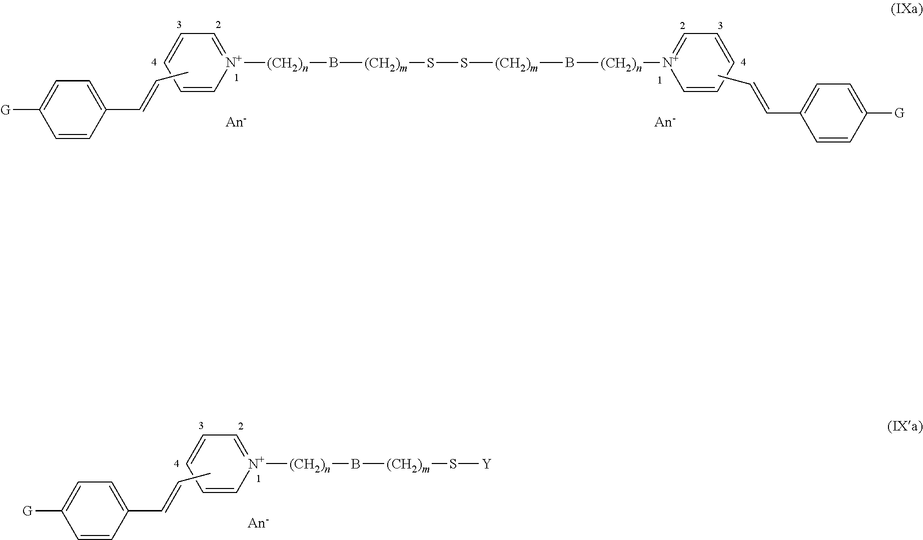

formulae (Ia) and (Ib) in which:
G represents R¹R²R³N⁺—, An⁻ as defined previously;
An⁻ represents an anionic counterion as defined previously;
B represent a divalent amido group —C(O)—N(R)— or —N(R)—C(O)—, with R representing a hydrogen atom or a (C₁-C₆)alkyl group; preferentially, R═H;
n and m, which may be identical or different, represent an integer between 1 and 4 inclusive; preferentially, n is equal to 3 and m is equal to 2;
Y is as defined previously;
   it being understood that the bond between the pyridinium ring and the double bond of the ethylene or styryl group is located in position 2 or 4 of the pyridinium, preferentially at 4.

As examples of disulfide, thiol and protected-thiol direct dyes of formula (I) of the invention, mention may be made of those having the following chemical structures:

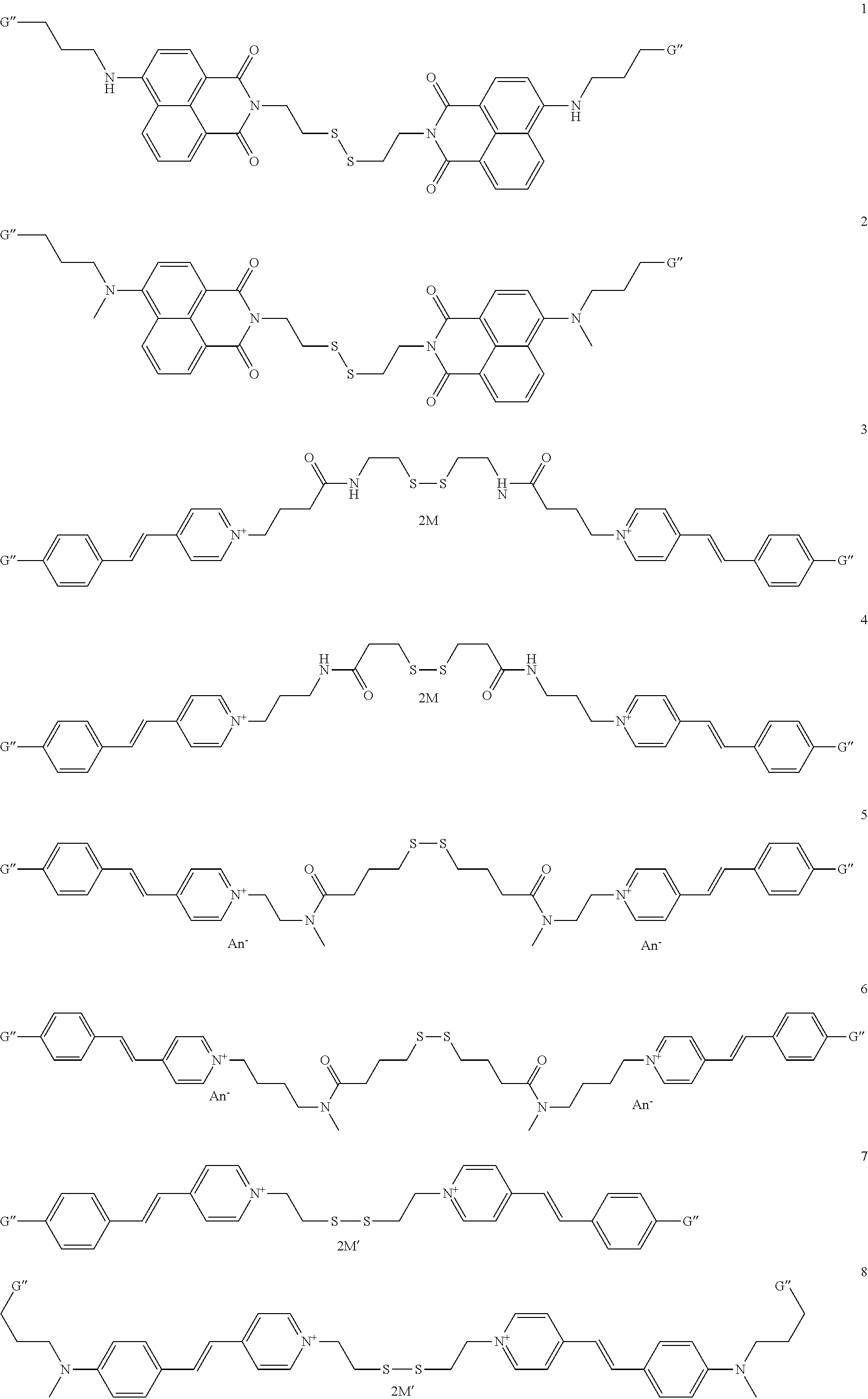

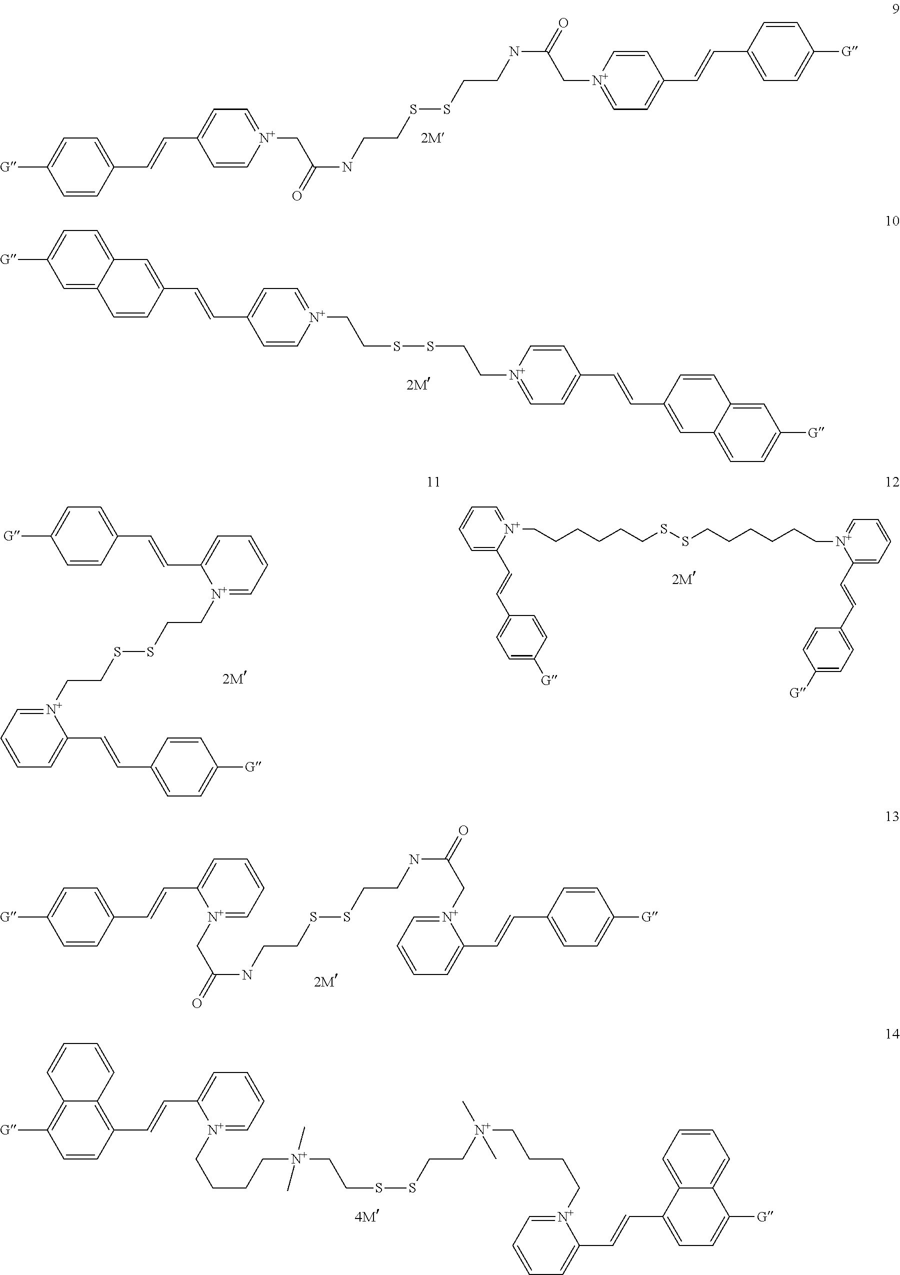

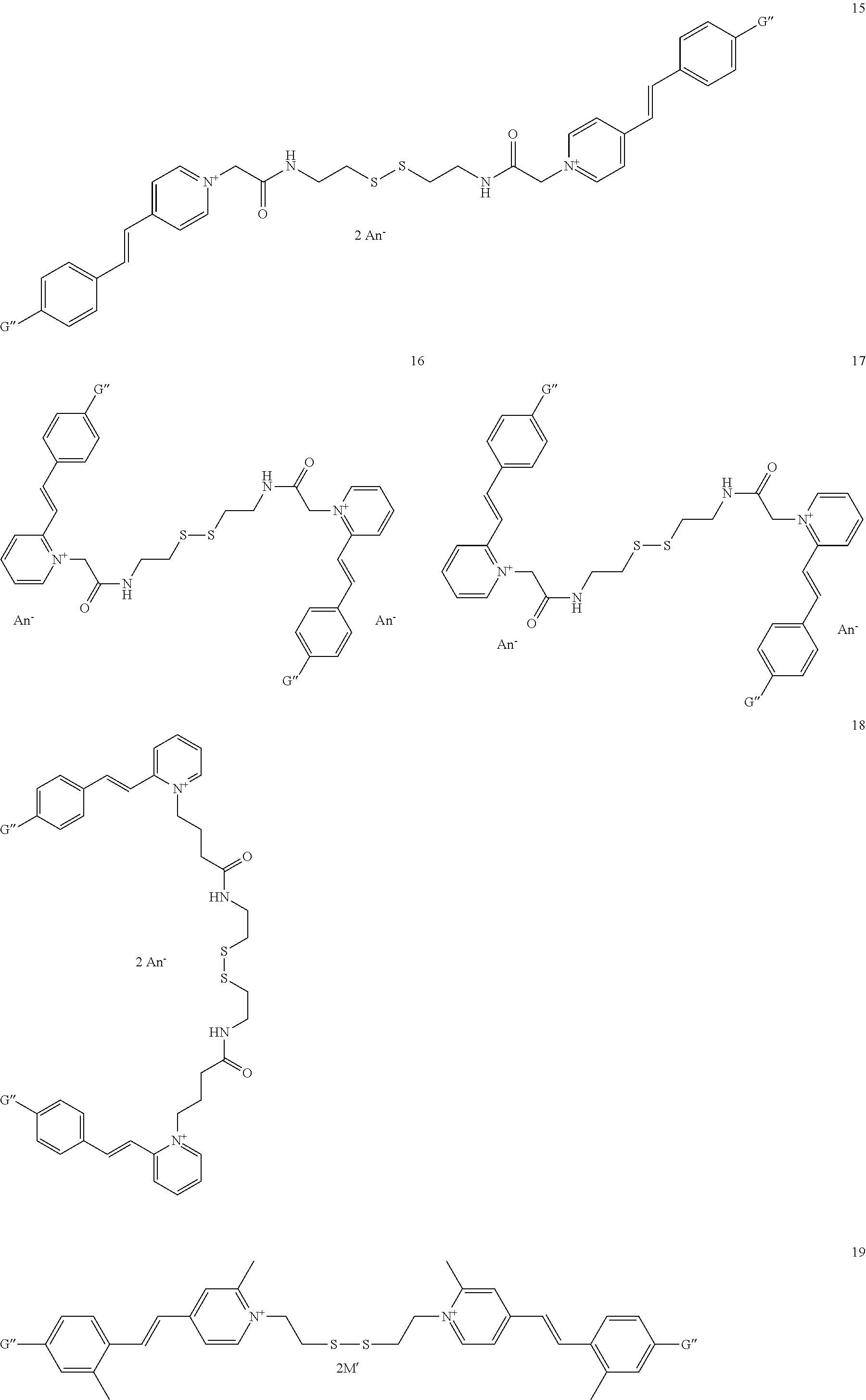

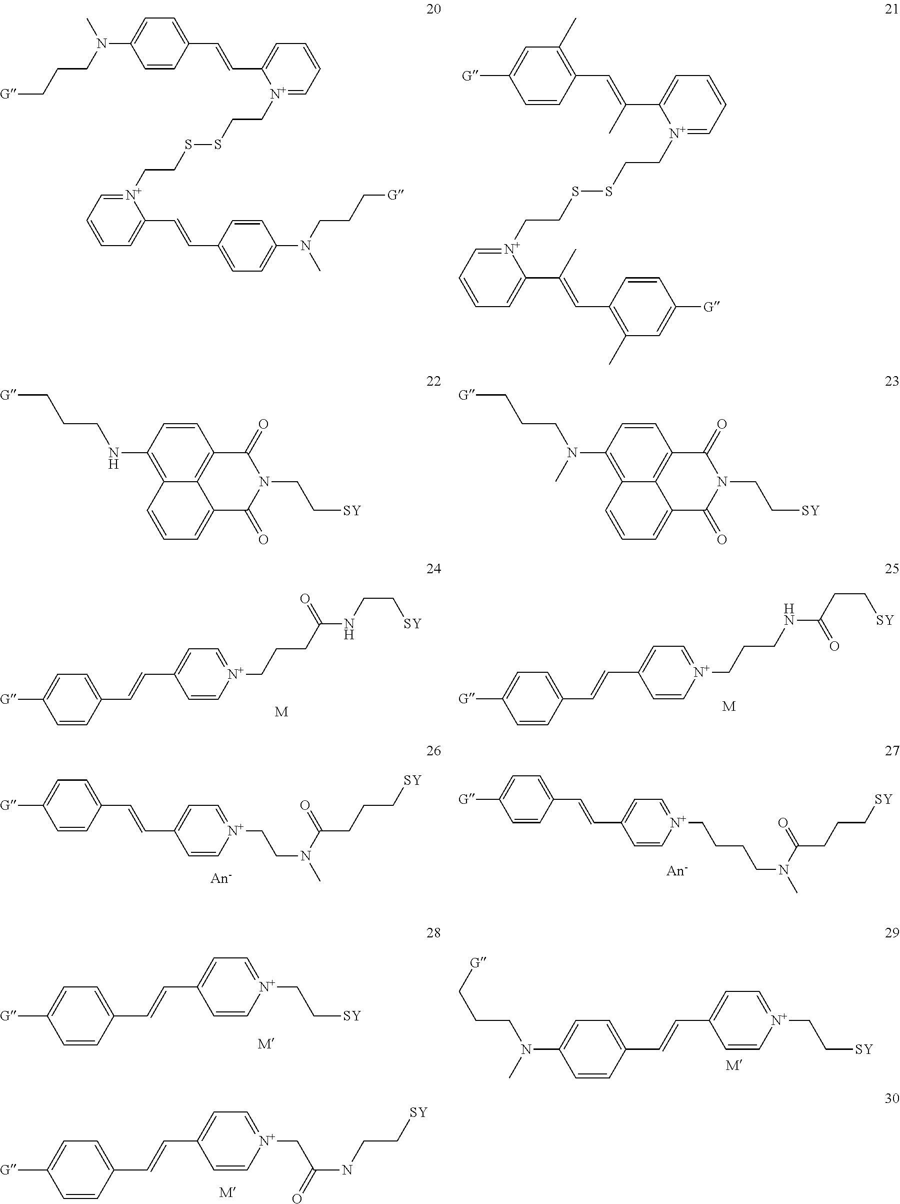

with An⁻ and M′, which may be identical or different, preferentially identical, representing anionic counterions; more particularly, the anionic counterion is chosen from halides such as chloride, alkyl sulfates such as methyl sulfate, and mesylate; Y is as defined previously and preferably represents a hydrogen atom or a group chosen from i) (C₁-C₄)alkylcarbonyl; ii) (C₁-C₄)alkylthiocarbonyl; iii) (C₁-C₄)alkoxycarbonyl; iv) (C₁-C₄)alkoxythiocarbonyl; v) (C₁-C₄)alkylthio-thiocarbonyl; vi) (di)(C₁-C₄)(alkyl)aminocarbonyl; vii) (di)(C₁-C₄)(alkyl)aminothiocarbonyl; viii) arylcarbonyl such as phenylcarbonyl; aryloxycarbonyl; ix) aryl(C₁-C₄)alkoxycarbonyl; x) (di)(C₁-C₄)(alkyl)aminocarbonyl such as dimethylaminocarbonyl; xi) (C₁-C₄)(alkyl)arylaminocarbonyl; xii) carboxyl; and G″ represents R¹R²R³N⁺—, An′⁻ with R¹ representing a linear or branched, preferably linear, (C₁₀-C₃₀)alkyl group; and R² and R³, which may be identical or different, represent a hydrogen atom or a (C₁-C₆)alkyl group, and An⁻ represents an organic or mineral anionic counterion such as a halide or an alkyl sulfate; in particular, R¹R²R³N⁺—, An⁻ represents n-C₁₅H₃₃—N⁺(CH₃)₂—, An′⁻, with An′⁻ as defined for An⁻ previously; in particular, An′⁻ represents a halide such as Cl⁻.

More particularly, the dye(s) of formula (I) of the invention are chosen from those below:

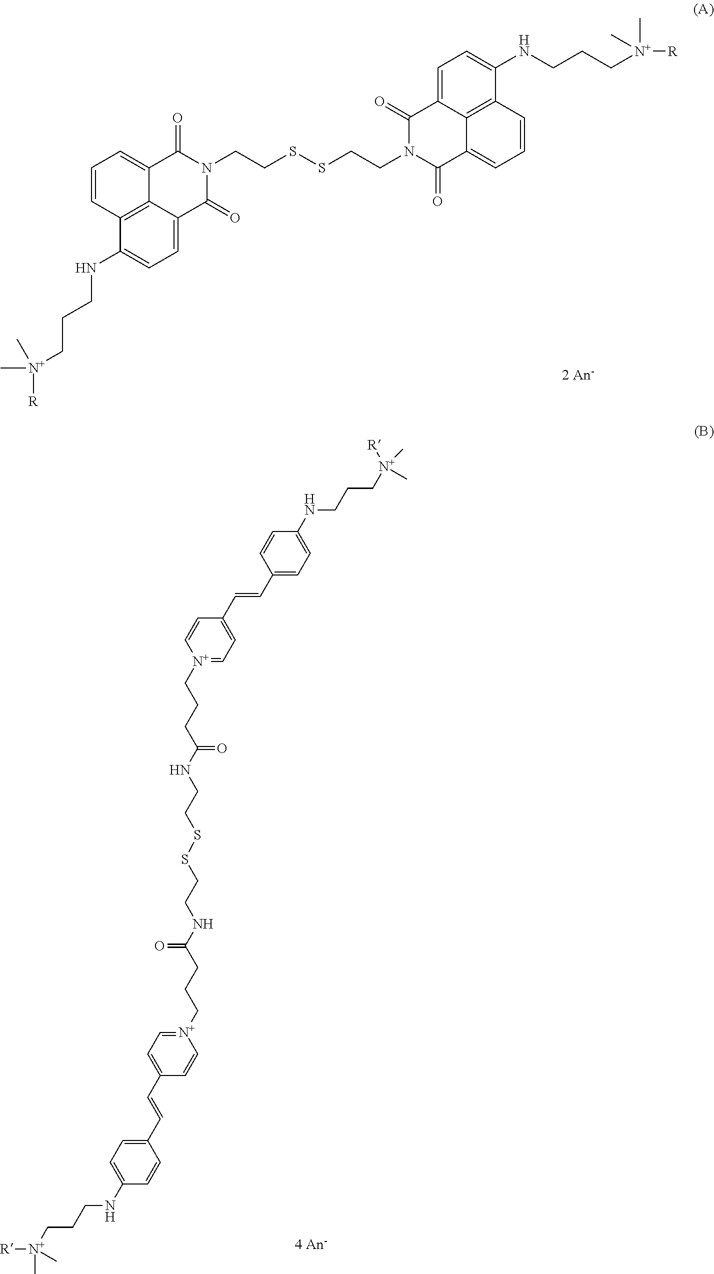

the organic or mineral acid salts and geometrical isomers thereof, and the solvates such as hydrates;
in which disulfide dyes (A) or (B):
R, which are identical, represent a linear (C₁₀-C₃₀)alkyl group, preferably chosen from n-C₁₄H₂₉—, n-C₁₆H₃₃— and n-C₁₈H₃₇—;
R′, which are identical, represent a linear (C₁₀-C₃₀)alkyl group, preferably chosen from n-C₁₄H₂₉—, n-C₁₆H₃₃— and n-C₁₈H₃₇—; and
An⁻, which may be identical or different, represent an anionic counterion as defined previously, in particular a halide such as Cl⁻ or Br⁻.

According to another particular embodiment, the fluorescent direct dye(s) of formula (I) are chosen from anionic dyes. According to this particular embodiment, A and/or A′ represent an anionic fluorescent chromophore comprising at least one linear or branched, saturated or unsaturated C₁₀-C₃₀ aliphatic chain; preferably, at least one of the two chromophores bears at least one ammonium group bearing a linear or branched, saturated or unsaturated C₁₀-C₃₀ aliphatic chain. Said chromophore(s) A and/or A′ are fluorescent and “derived” from anionic direct dyes commonly known as “acid dyes” on account of their affinity for alkaline substances (see, for example, Industrial Dyes, Chemistry, Properties, Application, Klaus Hunger Ed. Wiley-VCH Verlag GmbH & CoKGaA, Weinheim 2003). Anionic or acid dyes are known in the literature (see, for example, Ullman's Encyclopedia of Industrial Chemistry, Azo Dyes, 2005 Wiley-VCH Verlag GmbH & Co. KGaA, Weinheim 10.1002/14356007.a03 245, point 3.2; ibid, Textile Auxiliaries, 2002 Wiley-VCH Verlag GmbH & Co. KGaA, Weinheim 10.1002/14356007.a26 227 and Ashford's Dictionary of Industrial Chemicals, Second Edition, pages 14-39, 2001). The term “anionic direct dye” means any direct dye comprising in its structure at least one sulfonate SO₃⁻ group and/or at least one carboxylate group C(O)O⁻ and/or at least one phosphonate P(═O)O⁻O⁻ group and optionally one or more anionic groups G⁻ with G⁻, which may be identical or different, representing an anionic group chosen from alkoxide O⁻, thiolate S⁻, phosponate, carboxylate and thiocarboxylate: C(Q)Q′⁻, with Q and Q′, which may be identical or different, representing an oxygen or sulfur atom; preferably, G⁻ represents a carboxylate, i.e. Q and Q′ represent an oxygen atom. According to a particular embodiment:

The preferred anionic chromophores of the invention are chosen from chromophores derived from acidic azine direct dyes, acidic triarylmethane dyes, acidic indoamine dyes, acidic anthraquinone dyes, anionic styryl dyes and anionic triarylmethane direct dyes; each of these dyes bearing at least one sulfonate, phosphonate or carboxylate group bearing a cationic counterion X⁺ as defined previously; preferentially sulfonate or carboxylate of X⁺ with X⁺ representing a cationic counterion.

Anionic chromophores A and/or A′ that may particularly be mentioned include those having the following formulae:

a) the anthraquinone chromophores of formulae (XI) and (XI′):

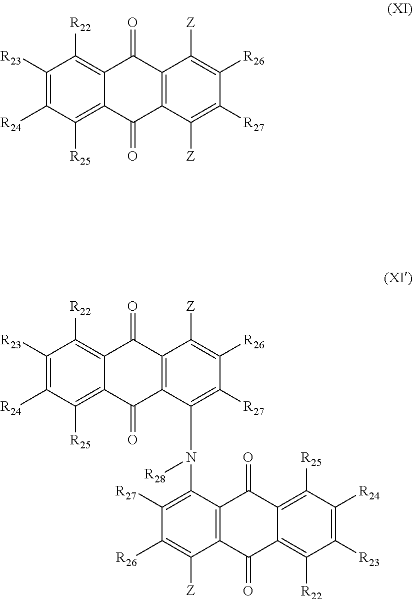

in which formulae (XI) and (XI′):
R₂₂, R₂₃, R₂₄, R₂₅, R₂₆ and R₂₇, which may be identical or different, represent a hydrogen or halogen atom or a group chosen from:
   alkyl;
   hydroxyl, mercapto;
   alkoxy, alkylthio;
   optionally substituted aryloxy or arylthio, preferentially substituted with one or more groups chosen from alkyl and (O)₂S(O⁻)—, X⁺ with X⁺ as defined previously;
   aryl(alkyl)amino optionally substituted with one or more groups chosen from alkyl and (O)₂S(O⁻)—, X⁺ with X⁺ as defined previously;
   (di)(alkyl)amino;
   (di)(hydroxyalkyl)amino;
   (O)₂S(O⁻)—, X⁺ with X⁺ as defined previously;
Z′ represents a hydrogen atom or a group NR₂R²₉ with R₂₈ and R₂₉, which may be identical or different, representing a hydrogen atom or a group chosen from:
   alkyl;
   polyhydroxyalkyl such as hydroxyethyl;
   aryl optionally substituted with one or more groups, particularly i) alkyl such as methyl, n-dodecyl, n-butyl; ii) (O)₂S(O⁻)—, X⁺ with X⁺ as defined previously; iii) R^{o}—C(X)—X′—, R^{o}—X′—C(X)—, R^{o}—X′—C(X)—X″— with R^{o}, X, X′ and X″ as defined previously; preferentially, R^{o} represents an alkyl group;
   cycloalkyl, especially cyclohexyl;
Z represents a group chosen from hydroxyl and NR′₂₈R′₂₉ with R′₂₈ and R′₂₉, which may be identical or different, representing the same atoms or groups as R₂₈ and R₂₉ as defined previously;
   it being understood that formulae (XI) and (XI′) comprise at least one sulfonate group (O)₂S(O⁻)—, X⁺ with X⁺ as defined previously and that the chromophores (XI) and (XI′) are connected to the rest of the molecule i) either via the carbon atom which bears R₂₂, R₂₃, R₂₄, R₂₅, R₂₆ and R₂₇, in which case the substituent in question (chosen from R₂₂, R₂₃, R₂₄, R₂₅, R₂₆ and R₂₇) borne by the carbon atom connected to the rest of the molecule is absent; ii) or via the nitrogen atom which bears R₂₈, R′₂₈ or R′₂₉; in which case the substituent (chosen from R₂, R′₂₈ and R′₂₉) borne by the nitrogen atom connected to the rest of the molecule is absent.

As examples of anionic chromophores of formula (XI), mention may be made of the salts derived from: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; and, as examples of chromophores of formula (XI′), mention may be made of those derived from Acid Black 48;

b) the triarylmethane chromophores of formula (XII):

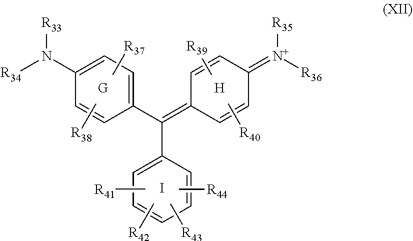

in which formula (XII):
R₃₃, R₃₄, R₃₅ and R₃₆, which may be identical or different, represent a hydrogen atom or a group chosen from alkyl, optionally substituted aryl and optionally substituted arylalkyl; particularly an alkyl group and benzyl optionally substituted with a group (O)ₘS(O⁻)—, X⁺ with X⁺ and m as defined previously;
R₃₇, R₃₈, R₃₉, R₄₀, R₄₁, R₄₂, R₄₃ and R₄₄, which may be identical or different, represent a hydrogen atom or a group chosen from:
   alkyl;
   alkoxy, alkylthio;
   (di)(alkyl)amino;
   hydroxyl, mercapto;
   nitro, nitroso;
   R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
   (O)₂S(O⁻)—, X⁺ with X⁺ as defined previously;
   (O)CO⁻—, X⁺ with X⁺ as defined previously;
   or alternatively two contiguous groups R₄₁ with R₄₂ or R₄₂ with R₄₃ or R₄₃ with R₄₄ together form a fused benzo group: I′: with I′ optionally substituted with one or more groups chosen from i) nitro; ii) nitroso; iii) (O)₂S(O⁻)—, X⁺; iv) hydroxyl; v) mercapto; vi) (di)(alkyl)amino; vii) R^{o}—C(X)—X′—; viii) R^{o}—X′—C(X)—; and ix) R^{o}—X′—C(X)—X″—; with M⁺, R^{o}, X, X′ and X″ as defined previously;
      particularly, R₃₇ to R₄₀ represent a hydrogen atom, and R₄₁ to R₄₄, which may be identical or different, represent a hydroxyl group or (O)₂S(O⁻)—, X⁺; and when R₄₃ with R₄₄ together form a benzo group, it is preferentially substituted with a group (O)₂S(O⁻)—; it being understood that at least one of the rings G, H, I or I′ comprises at least one sulfonate (O)₂S(O⁻)—, X⁺ or carboxylate (O)C(O⁻)—, X⁺ group;
      and that the chromophore (XII) is connected to the rest of the molecule i) either via the carbon atom which bears R₃, R₃₈, R₃₉, R₄₀, R₄₁, R₄₂, R₄₃ or R₄₄, in which case the substituent in question (chosen from R₃a, R₃₈, R₃₉, R₄₀, R₄₁, R₄₂, R₄₃ and R₄₄) borne by said carbon atom is absent, or alternatively ii) or via the nitrogen atom which bears R₃₃, R₃₄, R₃₅ or R₃₆, in which case the substituent (chosen from R₃₃, R₃₄, R₃₅ and R₃₆) borne by the nitrogen atom connected to the rest of the molecule is absent.

As examples of dyes of formula (XII), mention may be made of the salts derived from: Acid Blue 1; Acid Blue 3; Acid Blue 7, Acid Blue 9; Acid Violet 49 and Acid Green 50;

c) the xanthene chromophores of formula (XIII):

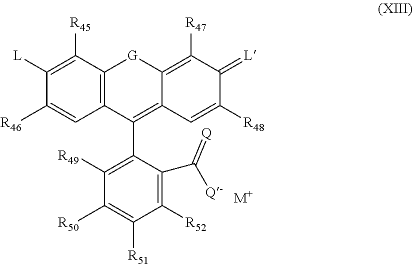

in which formula (XIII):
R₄₅, R₄₆, R₄₇ and R₄₈, which may be identical or different, represent a hydrogen atom or a halogen atom;
R₄₉, R₅₀, R₅₁ and R₅₂, which may be identical or different, represent a hydrogen or halogen atom, or a group chosen from:
   alkyl;
   alkoxy, alkylthio;
   hydroxyl, mercapto;
   nitro, nitroso;
   (O)₂S(O⁻)—, X⁺ with X⁺ as defined previously;
   (O)CO⁻—, X⁺ with X⁺ as defined previously;
      particularly, R₅₃, R₅₄, R₅₅ and R₄₈ represent a hydrogen or halogen atom;
G represents an oxygen or sulfur atom or a group NRₑ with Rₑ as defined previously; particularly, G represents an oxygen atom;
L represents an alkoxide O⁻, X⁺; a thioalkoxide S⁻, X⁺ or a group NR_{f}, with R_{f} representing a hydrogen atom or an alkyl group and X⁺ as defined previously;
L′ represents an oxygen or sulfur atom or an ammonium group: N⁺R_{f}R_{g}, with R_{f} and R_{g}, which may be identical or different, representing a hydrogen atom, an alkyl group or an optionally substituted aryl; L′ represents particularly an oxygen atom or a phenylamino group optionally substituted with one or more alkyl or (O)ₘS(O⁻)—, X⁺ groups with m and X⁺ as defined previously;
Q and Q′, which may be identical or different, represent an oxygen or sulfur atom; particularly Q and Q′ represent an oxygen atom;
X⁺ is as defined previously;
   it being understood that formula (XIII) comprises at least one sulfonate (O)₂S(O⁻)—, X⁺ or carboxylate (O)C(O⁻)—, X⁺ group with X⁺ as defined previously and that the chromophore (XIII) is connected to the rest of the molecule via the carbon atom which bears R₄₅, R₄₆, R₄₇, R₄₈ R₄₉, R₅₀, R₅₁ or R₅₂, in which case the substituent in question (chosen from R₄₅, R₄₆, R₄₇, R₄₈ R₄₉, R₅₀, R₅₁ and R₅₂) borne by said carbon atom is absent.

As examples of chromophores of formula (XIII), mention may be made of those derived from Acid Yellow 73; Acid Red 51; Acid Red 52; Acid Red 87; Acid Red 92; Acid Red 95; Acid Violet 9;

d) the quinoline chromophores of formula (XIV):

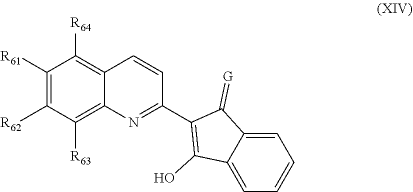

in which formula (XIV):
R₆₁ represents a hydrogen or halogen atom or an alkyl group;
R₆₂, R₆₃ and R₆₄, which may be identical or different, represent a hydrogen atom or a group (O)₂S(O⁻)—, X⁺ with X⁺ as defined previously;
or alternatively R₆₁ with R₆₂, or R₆ with R₆₄, together form a benzo group optionally substituted with one or more groups (O)₂S(O⁻)—, X⁺ with X⁺ as defined previously;
G represents an oxygen or sulfur atom or a group NRₑ with Rₑ as defined previously; particularly, G represents an oxygen atom;
   it being understood that formula (XIV) comprises at least one sulfonate group (O)₂S(O⁻)—, X⁺ with X⁺ as defined previously and that the chromophore (XIV) is connected to the rest of the molecule via the carbon atom which bears R₆₁, R₆₂, R₆₃ or R₆₄, in which case the substituent in question (chosen from R₆₁, R₆₂, R₆₃ and R₆₄) borne by said carbon atom is absent.

As examples of dyes of formula (XIV), mention may be made of the chromophores derived from the following dyes: Acid Yellow 2, Acid Yellow 3 and Acid Yellow 5.

Use may also be made of compounds corresponding to the mesomeric or tautomeric forms of structures (XI) to (XIV).

1.1.5 the Cosmetically Acceptable Organic or Mineral Acid Salt and Counterion of the Dyes of the Invention:

an “organic or mineral acid salt” is more particularly chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; xiv) triflic acid CF₃SO₃H; and xv) tetrafluoroboric acid HBF₄;

The term “anionic counterion” means an anion or an anionic group derived from an organic or mineral acid salt which counterbalances the cationic charge of the dye; more particularly, the anionic counterion is chosen from: i) halides such as chloride or bromide; ii) nitrates; iii) sulfonates, including C₁-C₆ alkylsulfonates: Alk-S(O)₂O⁻ such as methylsulfonate or mesylate and ethylsulfonate; iv) arylsulfonates: Ar—S(O)₂O⁻ such as benzenesulfonate and toluenesulfonate or tosylate; v) citrate; vi) succinate; vii) tartrate; viii) lactate; ix) alkyl sulfates: Alk-O—S(O)O— such as methyl sulfate and ethyl sulfate; x) aryl sulfates: Ar—O—S(O)O— such as benzene sulfate and toluene sulfate; xi) alkoxy sulfates: Alk-O—S(O)₂O⁻ such as methoxy sulfate and ethoxy sulfate; xii) aryloxy sulfates: Ar—O—S(O)₂O—, xiii) phosphates O═P(OH)₂—O—, O═P(O⁻)₂—OH, O═P(O⁻)₃, HO—[P(O)(O⁻)]_{w}—P(O)(O⁻)₂ with w being an integer; xiv) acetate; xv) triflate; and xvi) borates such as tetrafluoroborate, xvii) disulfate (O═)₂S(O⁻)₂ or SO₄²⁻ and monosulfate HSO₄⁻; the anionic counterion, derived from an organic or mineral acid salt, ensures the electrical neutrality of the molecule; thus, it is understood that when the anion comprises several anionic charges, then the same anion can serve for the electrical neutrality of several cationic groups in the same molecule or else may serve for the electrical neutrality of several molecules; for example, a disulfide dye of formula (I) which contains two cationic chromophores may contain either two “singly charged” anionic counterions or contains a “doubly charged” anionic counterion such as (O═)₂S(O⁻)₂ or O═P(O⁻)₂—OH;

the term “cationic counterion” means a cation or a cationic group derived from a salt of an organic or mineral base or basifying agent as defined below, counterbalancing the anionic charge of the dye; more particularly, the cationic counterion is chosen from alkali metals such as sodium or potassium, alkaline-earth metals such as calcium, and ammonium such as RR′R″R′″N⁺ with R, R′, R″ and R′″ which may be identical or different, representing a hydrogen atom or a (C₁-C₆)alkyl group which is optionally substituted, especially with one or more hydroxyl or amino groups.

Moreover, the addition salts that may be used in the context of the invention are in particular chosen from addition salts with a cosmetically acceptable base such as basifying agents as defined below, for instance alkali metal hydroxides, such as sodium hydroxide or potassium hydroxide, aqueous ammonia, amines or alkanolamines.

1.2. Preparation of the Direct Dyes Bearing a Disulfide or Thiol Function of the Invention:

The disulfide, thiol and protected-thiol dyes are prepared according to conventional methods known by those skilled in the art, EP 1 647 580, WO 2005/097051, EP 2 004 759, EP 2 075 289, WO 2007/110541, WO 2007/110540, WO 2007/110539, WO 2007/110538, WO 2007/110537, WO 2007/110536, WO 2007/110535, WO 2007/110534, WO 2007/110533, WO 2007/110532, WO 2007/110531, EP 2 070 988, WO 2009/040354 and WO 2009/034059.

It is in particular possible to prepare the disulfide, thiol or protected-thiol dyes of the invention (I) as defined previously using two equivalents of reagents comprising a chromophore and at least one nucleofugal group such as halo (a) and one equivalent of amino disulfide compound (b)

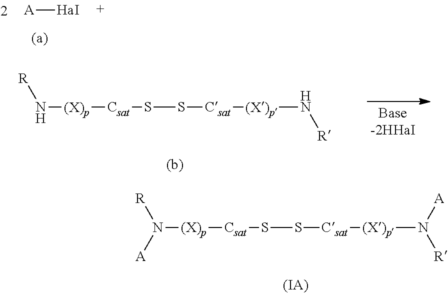

with Hal representing a nucleofugal group such as halo, (poly)halo(C₁-C₄)alkoxy, (poly)halo(C₁-C₄)sulfoxy, X, X′, p, p′, Cₛₐₜ and C′ₛₐₜ being as defined for the compound (I), R and R′ representing a hydrogen atom or an optionally substituted (C₁-C₄)alkyl group; A representing a chromophore as defined previously for (I) or else A represents a chromophore comprising a nucleofugal group as defined, which reacts with one or two equivalents of diaminoalkylene followed by an alkylation reaction using, for example, a (C₁₀-C₃₀)alkyl halide.

These reactions are preferably performed in a polar protic solvent, in particular at the reflux point of the solvent; preferably, the solvent is an alcohol such as ethanol.

According to another particular embodiment, the disulfide dyes of the invention are obtained by a divergent method: the first step consists in preparing the disulfide reagent (c) onto which will be grafted a heteroaryl substituted with at least one (C₁-C₆)alkyl group such as a pyridine substituted with a methyl group (d) which in turn reacts with an aryl(thio)aldehyde reagent comprising at least one ammonium group bearing a linear or branched, saturated or unsaturated C₁₀-C₃₀ aliphatic chain; with elimination of H₂O or H₂S so as to give a disulfide dye bearing a styryl chromophore (I′), and also the optical and geometric isomers thereof, belonging to formula (I) according to the invention:

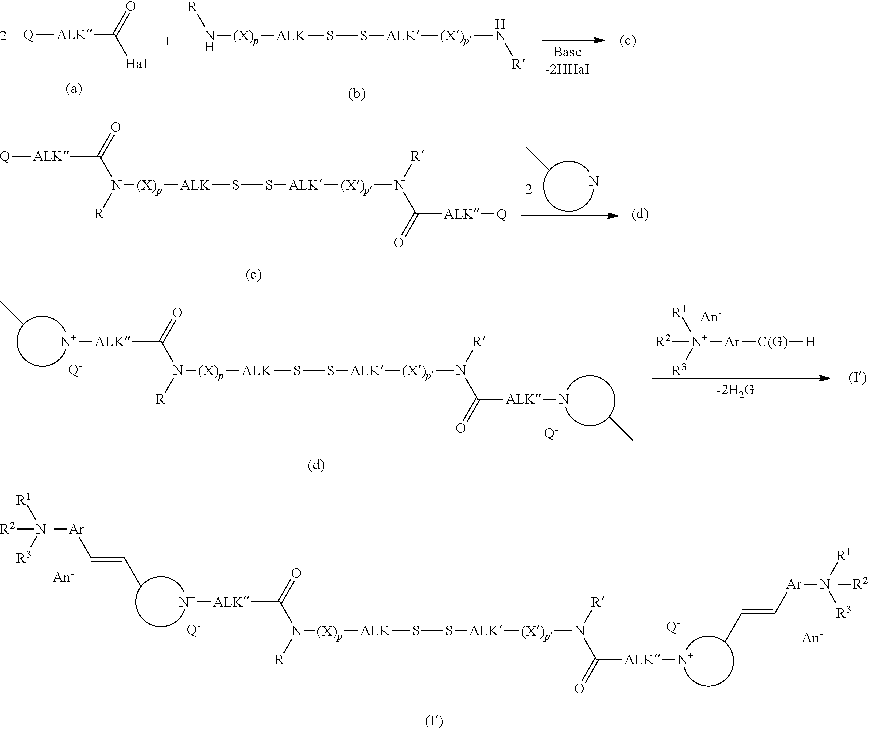

### Compounds (a), (b), (c), (d) and (1′):

Q represents an electrofugal group such as halo, (poly)halo(C₁-C₆)—S(O)₂—O—, alkyl sulfonyl (C₁-C₆)Alk-S(O)₂O— such as methylsulfonyl and ethylsulfonyl; arylsulfonyls: Ar—S(O)₂O⁻ such as benzenesulfonyl and toluenesulfonyl; (poly)(hydroxy)(C₁-C₆)alkylcarbonyloxy, alkylsulfonyl: Alk-O—S(O)O—; arylsulfonyls: Ar—O—S(O)O— such as benzenesulfonyl and toluenesulfonyl; alkoxysulfonyls: Alk-O—S(O)₂O—; aryloxysulfates: Ar—O—S(O)₂O—, phosphonyl; and borates such as tetrahaloborate;
Q⁻ represents an anionic counterion which derives from Q as defined above;
ALK, ALK′, ALK″, which may be identical or different, represent an optionally substituted linear or branched (C₁-C₆)alkylene group, such as ethylene or propylene;
Hal, X, X′, p and p′ are as defined above;
G represents an oxygen or sulfur, preferably oxygen, atom;
R′R²R³N⁺—, An⁻ is as defined previously, with R¹ representing a linear or branched, preferably linear, (C₁₀-C₃₀)alkyl group; and R² and R³, which may be identical or different, representing a hydrogen atom or a (C₁-C₆)alkyl group, and An representing an organic or mineral anionic counterion as defined previously, especially halide or an alkyl sulfate; in particular, R¹R²R³N⁺—, An⁻ represents n-C₁₅H₃₃—N⁺(CH₃)₂—, An⁻, with An⁻ as defined previously; in particular An⁻ represents a halide such as Cl⁻;

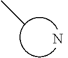

represents a heteroaryl group substituted with at least one (C₁-C₆)alkyl group, preferably methyl;

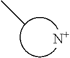

represents a cationic heteroaryl group substituted with at least one (C₁-C₆)alkyl group, preferably methyl;
Ar represents an optionally substituted aryl group, preferably phenyl.

1.3. The Composition of the Dyeing Process

The dye(s) bearing a disulfide, thiol or protected-thiol function of formula (I) as defined previously may be applied directly to keratin fibres in powder form or may be in a liquid composition.

The dye composition that is useful then contains, in a cosmetically acceptable medium, an amount of dye(s) bearing a disulfide, thiol or protected-thiol function t of formula (I) as defined previously generally of between 0.001% and 30% relative to the total weight of the composition.

Preferably, the amount of dyes bearing a disulfide, thiol or protected-thiol function of formula (I) as defined previously is between 0.01% and 5% by weight relative to the total weight of the composition. By way of example, the dye(s) are in an amount of between 0.01% and 2%.

Preferably, the composition of the dyeing and/or lightening process of the invention is in liquid form and contains one or more cationic fluorescent direct dyes of formula (I) bearing a disulfide function as defined previously.

The Medium:

The medium that is suitable for dyeing, also known as the dye support, is a cosmetic medium generally formed from water or a mixture of water and one or more organic solvents or a mixture of organic solvents.

The term “organic solvent” means an organic substance that is capable of dissolving another substance without chemically modifying it.

1.3.1 the Organic Solvents:

Examples of organic solvents that may be mentioned include lower C₁-C₄ alkanols, such as ethanol and isopropanol; polyols and polyol ethers, for instance 2-butoxyethanol, propylene glycol, propylene glycol monomethyl ether and diethylene glycol monoethyl ether and monomethyl ether, and also aromatic alcohols, for instance benzyl alcohol or phenoxyethanol, and mixtures thereof.

The solvents are preferably present in proportions preferably of between 1% and 40% by weight approximately and even more preferentially between 5% and 30% by weight approximately relative to the total weight of the dye composition.

1.3.2 the Adjuvants:

The composition comprising the dye(s) bearing a disulfide, thiol or protected-thiol function of formula (I) as defined previously may also contain various adjuvants conventionally used in hair dye compositions, such as anionic, cationic, nonionic, amphoteric or zwitterionic surfactants or mixtures thereof, anionic, cationic, nonionic, amphoteric or zwitterionic non-thiol and siliceous polymers or mixtures thereof, mineral or organic thickeners, and in particular anionic, cationic, nonionic and amphoteric polymeric associative thickeners, antioxidants, penetrants, sequestrants, fragrances, buffers, dispersants, conditioning agents, for instance volatile or non-volatile, modified or unmodified silicones, film-forming agents, ceramides, preserving agents and opacifiers.

The above adjuvants are generally present in an amount for each of them of between 0.01% and 20% by weight relative to the weight of the composition.

Needless to say, a person skilled in the art will take care to select this or these optional additional compound(s) such that the advantageous properties intrinsically associated with the dye composition in accordance with the invention are not, or are not substantially, adversely affected by the envisaged addition(s).

1.3.3 the Additional Dyes:

The composition comprising the fluorescent direct dye(s) bearing a disulfide, thiol or protected-thiol function of formula (I) as defined previously may also contain one or more additional direct dyes other than the disulfide, thiol or protected-thiol fluorescent direct dyes of formula (I) according to the invention. These direct dyes are chosen, for example, from those conventionally used in direct dyeing, among which may be mentioned all the commonly used aromatic and/or non-aromatic dyes, such as neutral, acidic or cationic nitrobenzene direct dyes, neutral, acidic or cationic azo direct dyes, natural direct dyes, neutral, acidic or cationic quinone and in particular anthraquinone direct dyes, azine, triarylmethane or indoamine direct dyes, methines, styryls, porphyrins, metalloporphyrins, phthalocyanines and methinecyanines.

Among the natural direct dyes, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions comprising these natural dyes and in particular henna-based poultices or extracts.

According to the invention, the additional direct dye(s) used according to the invention preferably represent from 0.001% to 10% by weight approximately relative to the total weight of the dye composition comprising the fluorescent direct dye(s) bearing a disulfide, thiol or protected-thiol function of formula (as defined previously and even more preferentially from 0.05% to 5% by weight approximately.

The composition comprising the fluorescent direct dye(s) bearing a disulfide, thiol or protected-thiol function of formula (I) as defined previously may also contain one or more oxidation bases and/or one or more couplers conventionally used for dyeing keratin fibres.

Among the oxidation bases, mention may be made of para-phenylenediamines, bis(phenyl)alkylenediamines, para-aminophenols, bis-para-aminophenols, ortho-aminophenols and heterocyclic bases, and the addition salts thereof.

Among these couplers, mention may be made especially of meta-phenylenediamines, meta-aminophenols, meta-diphenols, naphthalene-based couplers and heterocyclic couplers, and the addition salts thereof.

The coupler(s) are each generally present in an amount of between 0.001% and 10% by weight and preferably between 0.005% and 6% by weight relative to the total weight of the dye composition.

The oxidation base(s) present in the dye composition are each generally present in an amount of between 0.001% and 10% by weight and preferably between 0.005% and 6% by weight relative to the total weight of the dye composition.

In general, the addition salts of the oxidation bases and couplers that may be used in the context of the invention are chosen especially from salts of addition with an acid, such as hydrochlorides, hydrobromides, sulfates, citrates, succinates, tartrates, lactates, tosylates, benzenesulfonates, phosphates and acetates, and salts of addition with a base, such as alkali metal hydroxides, for instance sodium hydroxide, potassium hydroxide, aqueous ammonia, amines or alkanolamines.

According to a particular embodiment, the composition of the process of the invention contains at least one oxidation base and optionally at least one coupler as defined above.

The process of the invention may also use another composition that comprises one or more chemical oxidizing agents. The term “chemical oxidizing agent” means chemical oxidizing agents other than atmospheric oxygen.

The chemical oxidizing agent may be chosen, for example, from hydrogen peroxide, urea peroxide, alkali metal bromates such as sodium bromate, persalts such as perborates and persulfates, and enzymes such as peroxidases and two-electron or four-electron oxidoreductases, for instance uricases, and four-electron oxidases such as laccases.

The use of hydrogen peroxide is particularly preferred.

The content of oxidizing agent is generally between 1% and 40% by weight relative to the weight of the composition and preferably between 1% and 20% by weight relative to the weight of the composition.

1.3.4 The pH:

The pH of the composition comprising the fluorescent direct dye(s) bearing a disulfide, thiol or protected-thiol function of formula (I) as defined previously according to the invention is generally between 2 and 12 approximately and preferably between 3 and 11 approximately. It may be adjusted to the desired value by means of acidifying or basifying agents usually used in the dyeing of keratin fibres, or alternatively using standard buffer systems.

The pH of the composition is preferentially between 6 and 9.

Among the acidifying agents, examples that may be mentioned include mineral or organic acids as defined previously, for instance hydrochloric acid, orthophosphoric acid, sulfuric acid, carboxylic acids, for instance acetic acid, tartaric acid, citric acid or lactic acid, and sulfonic acids.

Among the basifying agents, examples that may be mentioned include aqueous ammonia, alkali metal carbonates, alkanolamines such as monoethanolamine, diethanolamine and triethanolamine, and also derivatives thereof, sodium hydroxide, potassium hydroxide and the compounds of formula (a) below:

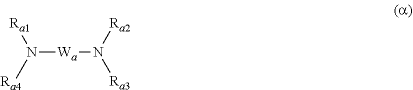

in which Wₐ is a linear or branched, preferentially linear, divalent (C₁-C₁₀)alkylene group, optionally interrupted with one or more heteroatoms such as O, S and NRₐ₁ and/or optionally substituted with one or more hydroxyl groups; Rₐ₁, Rₐ₂, Rₐ₃ and Rₐ₄, which may be identical or different, represent a hydrogen atom or a C₁-C₄ alkyl or C₁-C₄ hydroxyalkyl radical; preferentially, Wₐ represents a propylene group.

1.3.5 Forms of the Composition:

The dye composition comprising the fluorescent direct dye(s) bearing a disulfide, thiol or protected-thiol function of formula (I) as defined previously may be in various presentation forms, such as in the form of a liquid, a lotion, a cream or a gel, or in any other form that is suitable for dyeing keratin fibres. It may also be conditioned under pressure in an aerosol can in the presence of a propellant and form a mousse.

1.4. Mode of Application of the Dyes (I) Optionally in the Presence of a Heat Source

A subject of the invention is also a process for dyeing keratin materials, in particular keratin fibres such as the hair, using one or more fluorescent direct dyes of formula (I) as defined previously.

According to a particular embodiment of the invention, the process for dyeing keratin materials comprises i) the application to said materials, in particular keratin fibres such as the hair, preferably dark hair, of one or more fluorescent direct dyes bearing a disulfide, thiol or protected-thiol function of formula (I) as defined previously and ii) a heat source such as steam and/or an iron for straightening said fibres.

The process for treating keratin materials, in particular keratin fibres such as the hair, preferably dark hair, comprises the following steps:

i) applying to the hair fibres a dye composition comprising, in a cosmetic medium, one or more fluorescent direct dyes bearing a disulfide, thiol or protected-thiol function of formula (I) as defined previously,
ii) increasing the temperature of the hair fibres to a temperature of between 50 and 280° C., steps i) and ii) possibly being performed simultaneously or sequentially.

After applying the dye composition, and before increasing the temperature of the hair fibres, said composition may be left on, generally for 30 seconds to 60 minutes, preferably 5 to 45 minutes. The process according to the invention comprises, preferably after the step of applying the dye composition, a step of increasing the temperature of the hair fibres to a temperature between 100 and 250° C. Preferably, the step of heating the keratin fibres is performed at a temperature ranging from 150 to 220° C., preferably ranging from 160° C. to 220° C., preferentially ranging from 160° C. to 200° C., especially ranging from 170° C. to 190° C.

According to one particular embodiment of the invention, the temperature is increased by means of an iron.

For the purposes of the present invention, the term “iron” means a device for heating hair fibres which places said fibres and the heating device in contact.

The irons that can be used may be curling irons, straightening irons, crimping irons or steam irons.

As examples of irons that may be used in the process according to the invention, mention may be made of any type of flat or round irons, and in particular, in a non-limiting manner, those described in patents U.S. Pat. Nos. 4,103,145, 4,308,878, 5,983,903, 5,957,140, 5,494,058 and 5,046,516.

The end of the iron which comes into contact with the keratin fibres generally has two flat surfaces. These two surfaces may be made of metal or of ceramic. In particular, these two surfaces may be smooth or crimped or curved.

The iron may be applied by successive separate strokes lasting a few seconds or by gradual movement or sliding along the locks of keratin fibres, especially of hair.

In particular, the iron is applied in the process according to the invention by a continuous movement from the root to the end of the hair, in one or more passes, in particular in two to twenty passes. The duration of each pass of the iron may range from 2 seconds to 1 minute.

Preferably, the iron is applied in the process according to the invention by a continuous movement from the root to the end, in one or more passes.

The process according to the invention may also comprise an additional step of totally or partially pre-drying the hair fibres before the step of increasing the temperature, so as to prevent significant amounts of steam being given off, which might burn the stylist's hands and the individual's scalp. This predrying step may be performed, for example, by means of a dryer or of a drying hood at a temperature below 50° C., or else by drying in the open air.

The temperature increase may be simultaneous with the application of the composition according to the invention.

Preferably, the increase in temperature follows the application of the composition according to the invention.

The above embodiments may be implemented using a device comprising a heat source combined with a reservoir of cosmetic composition containing an aqueous composition such as water. By way of example, such a conditioning and application device may comprise:
a reservoir, which is heated or unheated,
an aqueous cosmetic composition, preferably water,
an application device,
a heating means placed on either side of the application device which makes it possible to heat the aqueous cosmetic composition to the desired temperature, simultaneously with or after its application. The heating means can thus act as an applicator.

According to one embodiment of the invention, the treatment according to the invention is combined with an existing treatment of the fibre.

Thus, in a first step, permanent-waving or oxidation dyeing or bleaching or shampooing or a styling product or alkaline relaxing can be applied and, in a second step, the described process of the invention is performed. The durability of the first treatment is thus reinforced.

According to one embodiment, the process according to the invention is performed on natural keratin fibres, especially natural hair.

According to one embodiment, the process according to the invention is performed on damaged keratin fibres, especially hair. As indicated previously, the term “damaged hair” means dry or coarse or brittle or split or limp hair.

In other words, the treatment process according to the invention is preferably performed on sensitized keratin fibres, in particular hair, such as bleached, relaxed or permanent-waved fibres.

The process according to the invention may be performed on keratin fibres, especially hair, which is wet or dry. Preferentially, the process is performed on dry keratin fibres, especially dry hair.

After application to the keratin fibres of the fluorescent direct dye(s) of formula (I) as defined previously, or of a cosmetic composition containing same, and before performing the step of heating the keratin fibres, the compound(s) of formula (I) or the composition containing same may be left on for a time ranging from 1 to 60 minutes, preferably ranging from 2 to 50 minutes and preferentially ranging from 5 to 45 minutes. The leave-on period may take place at a temperature ranging from 15° C. to 45° C., preferably at room temperature (25° C.).

The cosmetic composition described previously is advantageously applied to the keratin fibres in an amount ranging from 0.1 to 10 grams and preferably from 0.2 to 5 grams of composition per gram of keratin fibres.

After application of the cosmetic composition to the keratin fibres, said fibres may be wrung out to remove the excess composition or washed with water or with a shampoo.

The treatment process according to the invention may be performed before, during and/or after an additional process of cosmetic treatment of the keratin fibres, such as a process for temporarily shaping, such as shaping with curlers, a curling iron or a straightening iron, or a process for durably shaping, permanent-waving or relaxing the keratin fibres. Preferably, the dyeing process of the invention implements a post-treatment step consisting in applying a heat source, preferably using an iron for straightening the keratin fibres and/or steam, in particular originating from a steampod.

In particular, the treatment process according to the invention may be performed on damaged keratin fibres. The treatment process according to the invention is preferably performed on sensitized keratin fibres such as bleached, relaxed or permanent-waved fibres.

According to a particular embodiment of the invention, the step of applying or treating keratin fibres with steam is performed extemporaneously with that of the application or treatment of the keratin fibres with one or more fluorescent direct dyes bearing a disulfide, thiol or protected-thiol function of formula (I) as defined previously.

According to another particular embodiment of the process of the invention, the treatment of fibres with one or more fluorescent direct dyes bearing a disulfide, thiol or protected-thiol function of formula (I) as defined previously and the step of treating the keratin fibres are performed in two stages. In a first stage, the keratin fibres are treated with one or more fluorescent direct dyes bearing a disulfide, thiol or protected-thiol function of formula (I) as defined previously, and then, after a leave-on time, step ii) of straightening the keratin fibres with a hot iron is performed without intermediate rinsing. In particular, the fluorescent direct dye(s) bearing a disulfide, thiol or protected-thiol function of formula (I) as defined previously are in a dye composition as defined previously in liquid form (point 1.2). The leave-on time after application of the composition containing the fluorescent direct dyes of formula (I) is set at between 5 minutes and 2 hours. Preferentially it is between 15 minutes and 1 hour, such as 30 minutes.

The application of one or more fluorescent direct dyes bearing a disulfide, thiol or protected-thiol function of formula (I) as defined previously is generally performed at room temperature. It may, however, be performed at temperatures ranging from 20 to 80° C. and preferentially between 20 and 60° C., and the keratin fibres are then subjected to a treatment with steam.

The fibres may be treated with an iron for straightening keratin fibres assisted with steam. These irons are those that may be obtained commercially or those of professionals.

Another means is to arrange the keratin fibres treated beforehand with at least one fluorescent direct dye bearing a disulfide, thiol or protected-thiol function of formula (I) according to step i) as defined previously, over a source of steam such as a kettle, a boiling water container or a steam iron, for example the commercially available irons such as Joico K-Pak ReconstRx Vapor Iron and Babyliss Pro230 Steam.

The treatment time of the keratin fibres with steam is between 5 minutes and 2 hours and preferentially between 15 minutes and 1 hour, such as 30 minutes.

According to another process for dyeing keratin fibres, the composition that comprises at least one fluorescent direct dye bearing a disulfide, thiol or protected-thiol function of formula (I) as defined previously is an aqueous composition, this composition being applied to the hair followed by application of a straightening iron that generates steam in-situ.

According to a variant of the process for dyeing keratin fibres, the composition that comprises at least one fluorescent direct dye bearing a disulfide, thiol or protected-thiol function of formula (I) as defined previously is applied to wet or damp hair, followed by application of a straightening iron that also generates steam in-situ.

Preferentially, the process for dyeing keratin fibres does not use a reducing agent.

A treatment with a chemical oxidizing agent may optionally be combined as a post-treatment. Any type of oxidizing agent that is conventional in the field as described previously may be used. Thus, it may be chosen from hydrogen peroxide, urea peroxide, alkali metal bromates, persalts such as perborates and persulfates, and also enzymes, among which mention may be made of peroxidases, 2-electron oxidoreductases such as uricases, and 4-electron oxygenases such as laccases. The use of hydrogen peroxide is particularly preferred. The duration of the optional post-treatment with an oxidizing agent is between 1 second and 40 minutes. It is preferably between 1 and 10 minutes.

Preferentially, the chemical oxidizing agent(s), when they are present in the dyeing process of the invention, are in very mild concentrations, i.e. less than or equal to 5% by weight and preferentially 1% by weight relative to the total weight of the mixture comprising the fluorescent direct dye(s) bearing a disulfide, thiol or protected-thiol function of formula (I) as defined previously and the chemical oxidizing agent(s). According to a particular embodiment of the invention, the dyeing process does not use a chemical oxidizing agent.

The application of the composition may be performed on dry hair or may be preceded by moistening of the hair.

According to a particular embodiment of the dyeing process, it is sought to lighten dark keratin fibres, especially with a tone depth of less than or equal to 6 and preferentially less than or equal to 4. To do this, the process for the dyeing and optical lightening of dark keratin fibres uses i) one or more fluorescent direct dyes bearing a disulfide, thiol or protected-thiol function of formula (I) as defined previously, and ii) steam and/or a straightening iron applied to the keratin fibres; step ii) of treating with steam being performed either simultaneously or in a subsequent step after implementing a leave-on time between step i) and step ii) as mentioned previously.

Preferentially, the chemical oxidizing agent(s), when they are present in the lightening process according to the invention, are in very mild concentrations, i.e. less than or equal to 5% by weight and preferentially 1% by weight relative to the total weight of the mixture comprising the fluorescent direct dye(s) bearing a disulfide, thiol or protected-thiol function of formula (I) as defined previously and the chemical oxidizing agent(s). According to a particular embodiment of the invention, the dyeing process does not use a chemical oxidizing agent.

In particular, the dyeing and/or lightening process of the invention that uses i) the fluorescent direct dye(s) bearing a disulfide, thiol or protected-thiol function of formula (I) as defined previously and ii) steam under conditions as presented previously or a straightening iron applied to the keratin fibres is performed without using a reducing agent.

The examples that follow serve to illustrate the invention without, however, being limiting in nature.

The direct thiol, protected-thiol or disulfide dyes of formula (I) that are useful in the present invention are known compounds and may be prepared according to methods known to those skilled in the art, especially from the methods described in patent applications EP 1 647 580, EP 2 004 759, WO 2007/110 541, WO 2007/110 540, WO 2007/110 539, WO 2007/110 538, WO 2007/110 537, WO 2007/110 536, WO 2007/110 535, WO 2007/110 534, WO 2007/110 533, WO 2007/110 532, WO 2007/110 531, EP 2 070 988 and WO 2009/040 354.

### EXAMPLES

### Example A: Disulfide Dyes Bearing a Naphthalimide Chromophore of Formula (A) Below

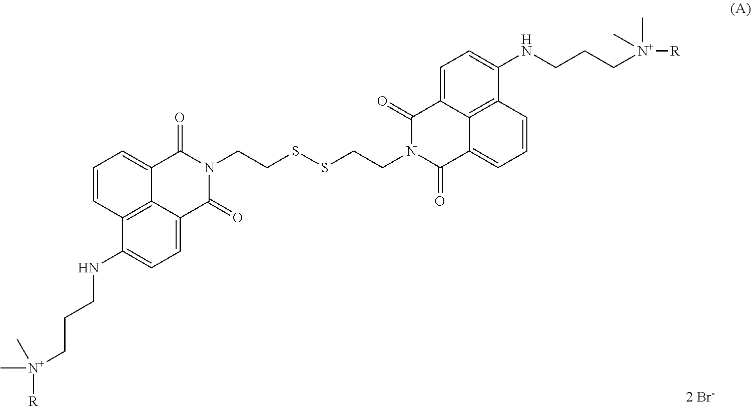

General synthetic route for obtaining the naphthalimide dyes (A) according to the invention:

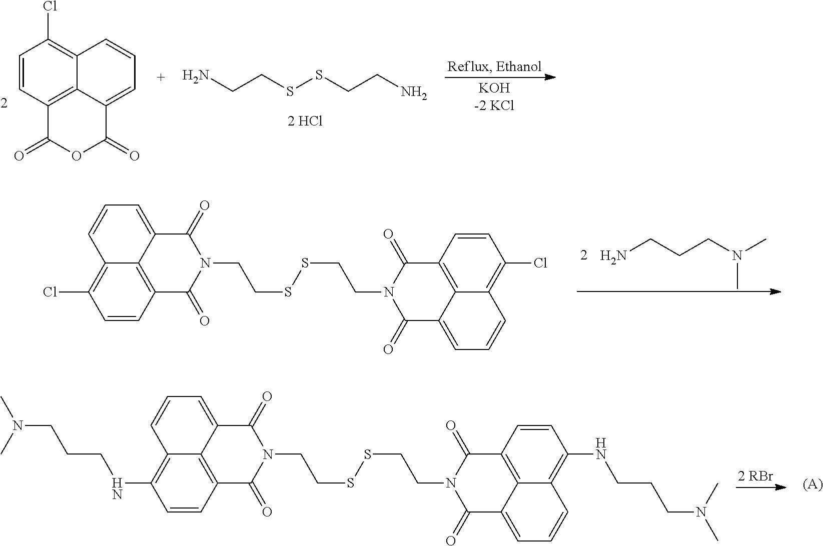

Example 1—Disulfide dye (A) for which R=n-C₁₄H₂₉—
Example 2—Disulfide dye (A) for which R=n-C₁₆H₃₃—
Example 3—Disulfide dye (A) for which R=n-C₁₈H₃₇—

### Example B: Disulfide Dyes Bearing a Hemicyanine/Styryl Chromophore of Formula (B) Below

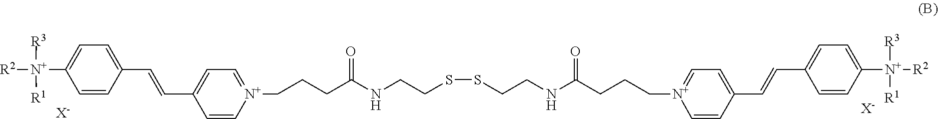

General synthetic route for obtaining the hemicyanine or styryl dyes (B) according to the invention:

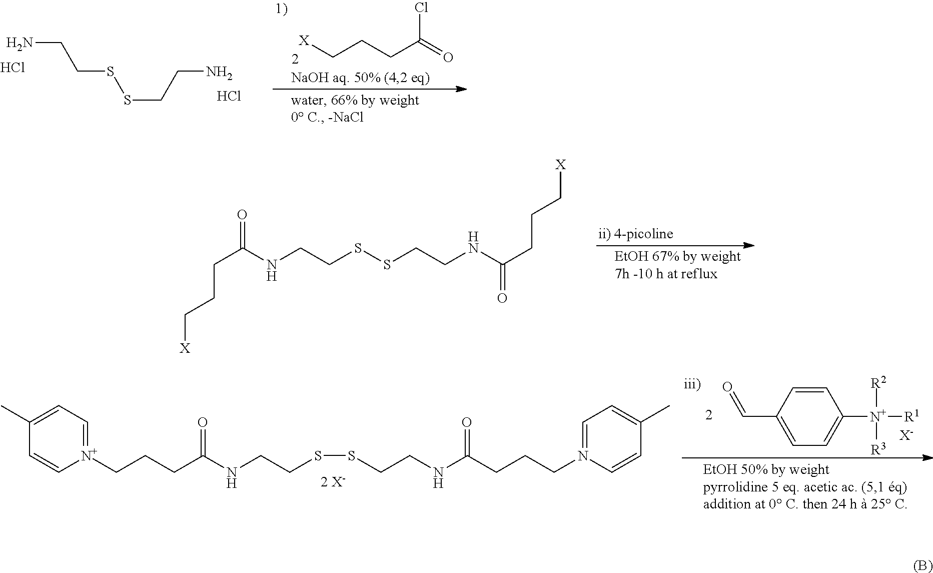

with R¹ and R² representing a methyl group and R³ representing a linear or branched C₁ to C₂₂ hydrocarbon-based group, and X⁻, which may be identical or different, represent an anionic counterion, in particular a halide such as chloride or bromide,

This synthetic route allows the access to compounds (B′) if 2-picoline is added in step ii):

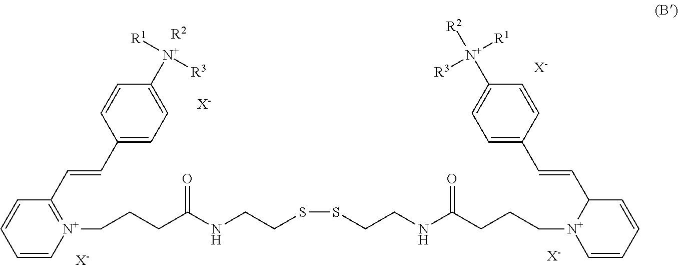

Example 4—Disulfide dye (B) for which R³=n-C₁₄H₂₉—
Example 5—Disulfide dye (B) for which R³=n-C₁₆H₃₃—
Example 6—Disulfide dye (B) for which R³=n-C₁₈H₃₇—
Example 7—Disulfide dye (B′) for which R³=n-C₁₄H₂₉—
Example 8—Disulfide dye (B′) for which R³=n-C₁₆H₃₃—
Example 9—Disulfide dye (B′) for which R³=n-C₁₈H₃₇—

### Dyes (A):

### Example 1

This compound is synthesized in three steps according to the following reaction scheme:

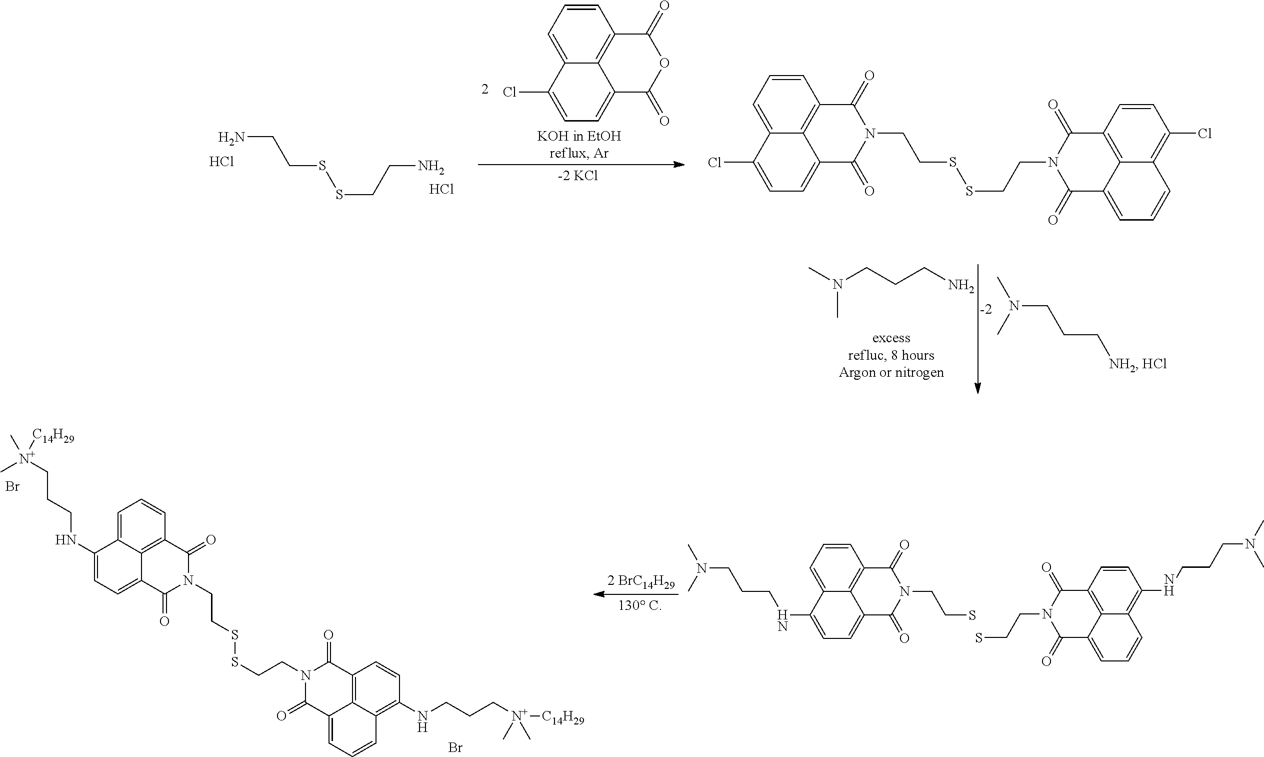

### Example 1: 3,3′-{Disulfanediylbis[ethane-2,1-diyl(1,3-dioxo-1H-benzo[de]isoquinoline-2,6(3H)-diyl)imino]}bis(N,N-dimethyl-N-tetradecylpropan-1-aminium) dibromide

### Step 1: Synthesis of 6-chloro-2-(2-{[2-(6-chloro-1,3-dioxo-1H-benzo[de]isoquinolin-2(3H)-yl)ethyl]disulfanyl}ethyl)-1H-benzo[de]isoquinoline-1,3(2H)-dione

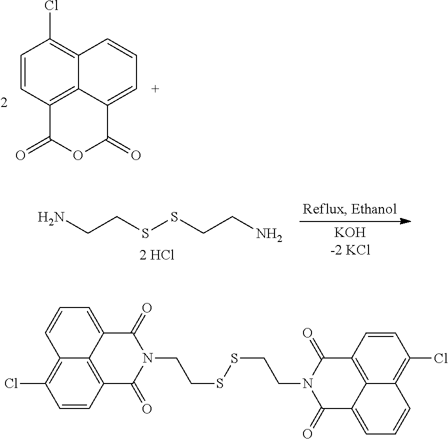

### Procedure:

96.8 g of cystamine dihydrochloride (0.430 mol) and then 860 ml of a solution, prepared beforehand, containing 1 mol/l of potassium hydroxide (0.860 mol) in ethanol (48.16 g) are introduced into a 1.5 litre Pignat reactor equipped with a condenser, an argon or nitrogen inlet and a mechanical stirring system. The reaction medium is then brought to 50° C. and maintained at this temperature for 30 minutes. 200 g of 4-chloro-1,8-naphthalic anhydride (0.860 mol) are then added, using a powder funnel, and the medium is then maintained at the reflux point of ethanol for 8 hours while monitoring the reaction progress by TLC (60 F 254 plate): 9/1 dichloromethane/methanol eluent, UV revelation. The medium is then allowed to return to room temperature overnight. The reaction medium is then cooled in a bucket containing 2 litres of water+ice and is kept stirring for 2 hours. A beige-coloured precipitate is then obtained, which is filtered off on a sinter funnel and rinsed thoroughly with 1 litre of cold water. The precipitate is dried under vacuum at 40° C. in the presence of P₂O₅ to constant weight, to obtain the expected compound in the form of a beige-coloured powder. On the basis of the NMR and mass spectrometry, the chemical structure is in accordance with the expected product.

### Step 2: Synthesis of 6-{[3-(dimethylamino)propyl]amino}-2-[2-({2-[6-{[3-(dimethylamino)propyl]amino}-1,3-dioxo-1H-benzo[de]isoquinolin-2(3H)-yl]ethyl}disulfanyl)ethyl]-1H-benzo[de]isoquinoline-1,3(2H)-dione

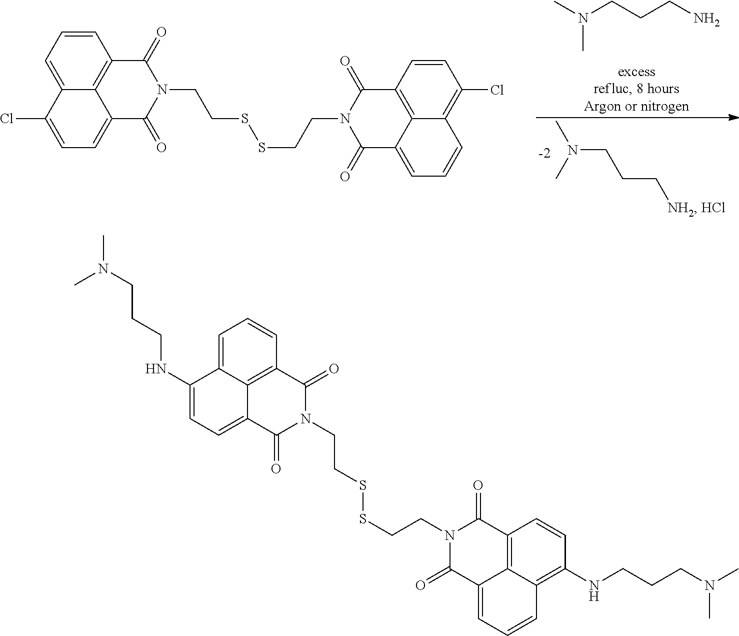

### Procedure:

200 g of the compound synthesized in the preceding step 1 (0.344 mol) and 780 ml of N,N dimethyl-1,3-propanediamine (6.191 mol) are placed in a 2 litre four-necked flask equipped with a condenser, an argon inlet and a magnetic stirring system. The reaction medium is maintained at 110° C. with stirring for 8 hours, while monitoring the reaction progress by TLC (60 F 254 plate): 9/1 dichloromethane/methanol eluent, UV revelation. Once the reaction is complete, the medium is allowed to return to room temperature and is cooled in a beaker containing 3 litres of water+ice kept stirring for 2 hours at room temperature. The medium is then filtered through a sinter and then slurried twice with 2×1 litre of ethanol. After drying under vacuum in the presence of P₂O₅ at 40° C. to constant weight, orange-brown crystals are obtained. These crystals are taken up with vigorous stirring in 2 litres of hot isopropanol maintained at reflux for 4 hours. The medium is then hot-filtered (40° C.) through a No. 3 sinter and then dried in a vacuum oven to constant weight to obtain the expected product in the form of an orange-yellow powder. The mass spectrometry and NMR analyses are in accordance with the expected product.

### Step 3: Synthesis of 3,3′-{disulfanediylbis[ethane-2,1-diyl(1,3-dioxo-1H-benzo[de]isoquinoline-2,6(3H)-diyl)imino]}bis(N,N-dimethyl-N-tetradecylpropan-1-aminium) dibromide

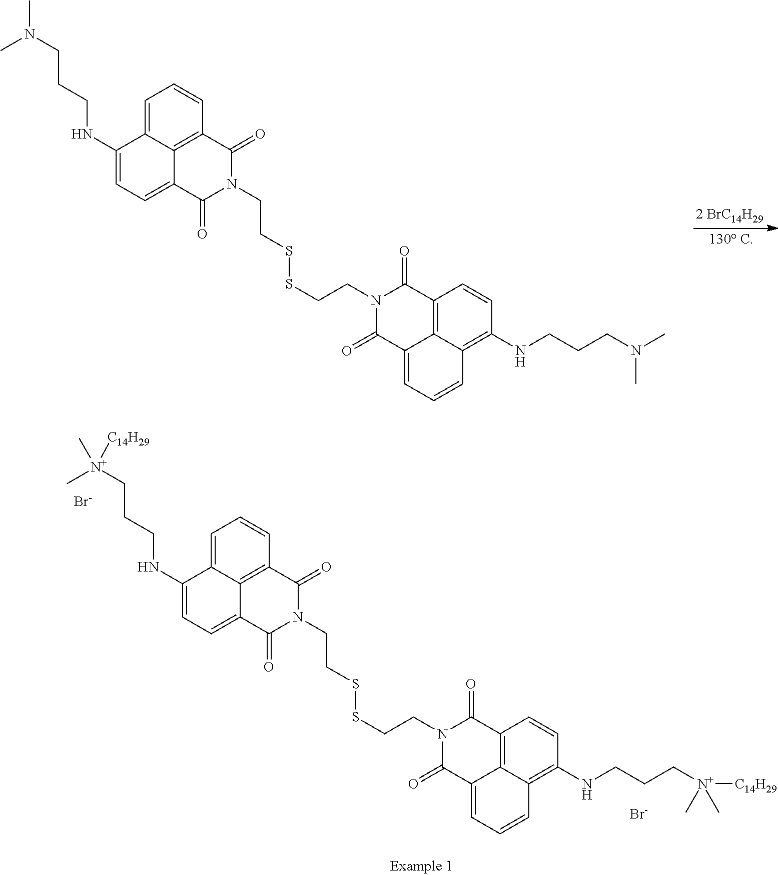

### Procedure:

5 g of the compound synthesized in the preceding step 2 (0.007 mol) are placed in a 1 litre four-necked flask equipped with a condenser, an argon or nitrogen inlet and a magnetic stirring system. 25 g of bromotetradecane (0.09 mol) are then added, the reaction medium is maintained at 130° C. for 24 hours and then maintained at room temperature with stirring for 2 hours. The reaction medium is then filtered through a No. 3 sinter, and then rinsed with 100 ml of acetone. A precipitate is obtained in the form of a yellow powder which is in accordance with the expected product on the basis of the spectroscopic analyses.

By following the same process, and changing just the alkylating agent in the final step, the following compounds are obtained:

### Example 2: 3,3′-{Disulfanediylbis[ethane-2,1-diyl(1,3-dioxo-1H-benzo[de]isoquinoline-2,6(3H)-diyl)imino]}bis(N,N-dimethyl-N-hexadecylpropan-1-aminium) dibromide

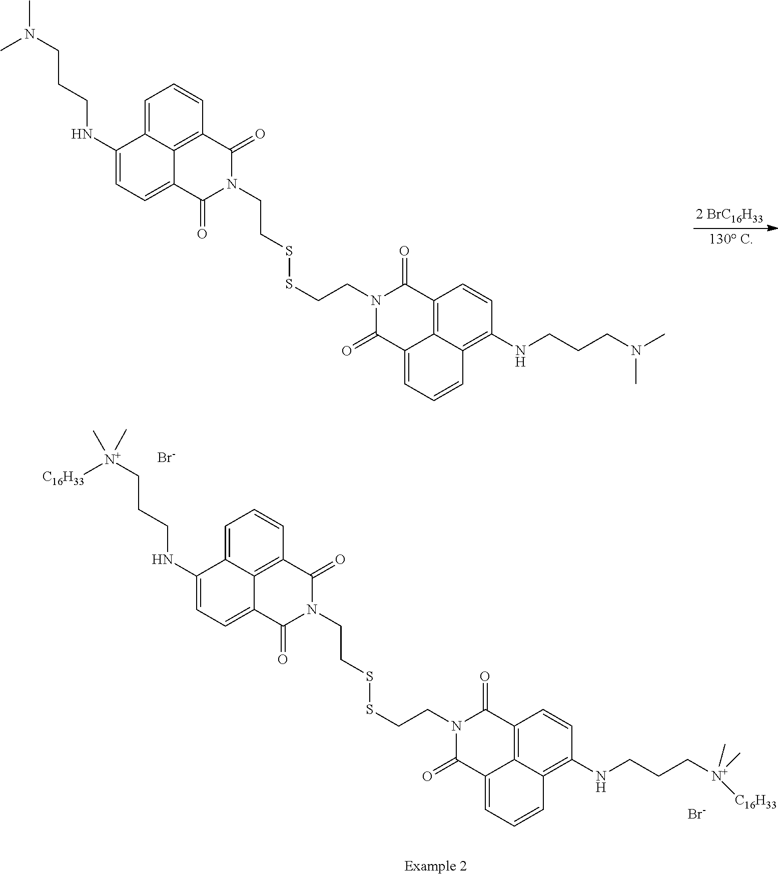

### Example 3: 3,3′-{Disulfanediylbis[ethane-2,1-diyl(1,3-dioxo-1H-benzo[de]isoquinoline-2,6(3H)-diyl)imino]}bis(N,N-dimethyl-N-octadecylpropan-1-aminium) dibromide

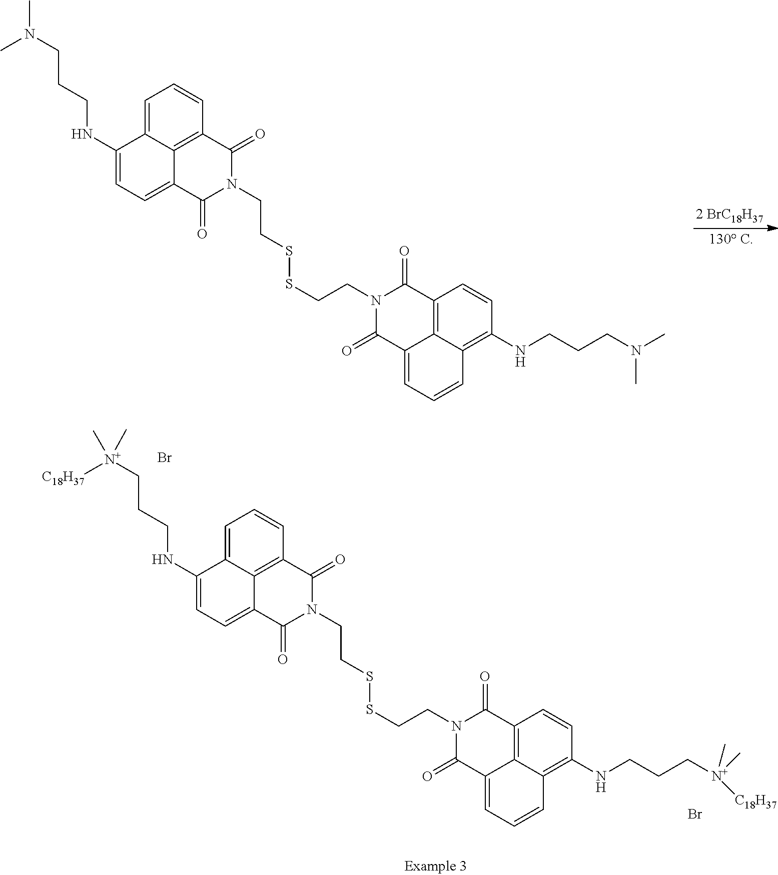

### Dyes (B:

### Example 4: 1,1′-{disulfanediylbis[ethane-2,1-diylimino(4-oxobutane-4,1-diyl)]}bis(4-{(2-[4-(dimethyltetradecylaminium)phenyl]ethenyl}pyridinium) tetrachloride

This compound is synthesized in three steps according to the following reaction scheme:

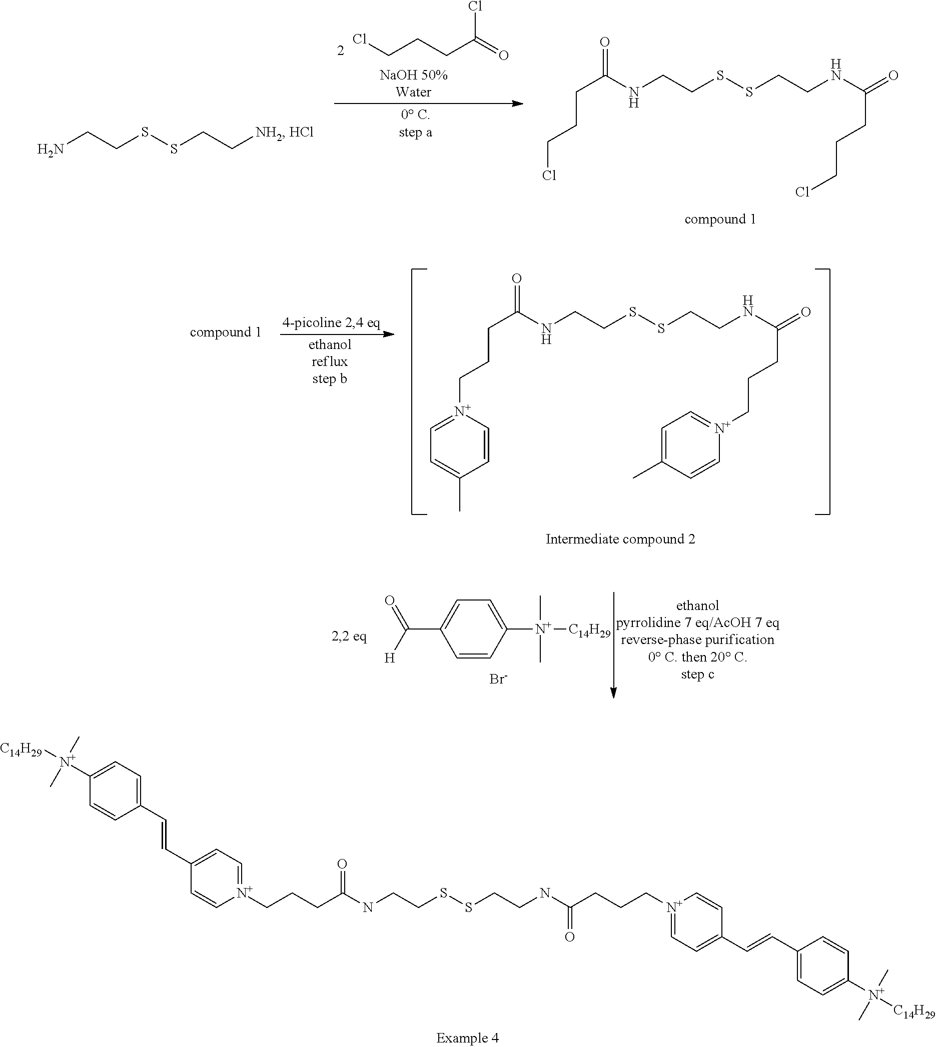

With X⁻, identical or different, which represents Cl⁻ or Br⁻ or a mixture of Cl⁻ and Br⁻.

### Step a): Synthesis of compound 1—N,N′-(disulfanediyldiethane-2,1-diyl)bis(4-chlorobutanamide)

100 g of 50% sodium hydroxide solution cooled to 0° C. are placed in a 1 litre four-necked flask equipped with a condenser, an argon or nitrogen inlet and a magnetic stirring system. A solution, prepared beforehand, of 69.9 g of cystamine hydrochloride dissolved in 150 ml of demineralized water is then added dropwise over 1 hour 30 minutes, so as to keep the temperature below 6° C. The reaction medium is then kept stirring for 1 hour at 5° C. 92.2 g of 4-chlorobutyryl chloride are then added dropwise, so as to keep the temperature below 5° C. Once the addition is complete, the reaction medium is allowed to return to room temperature. The reaction medium is then filtered through a sinter and the precipitate formed is washed with 3×100 ml of water and then with 3×100 ml of isopropanol and dried under vacuum at 40° C. to constant weight. The NMR analyses are in accordance with the expected compound 1.

### Step b): Synthesis of compound 2—1,1′-{disulfanediylbis[ethane-2,1-diylimino(4-oxobutane-4,1-diyl)]}bis(4-methylpyridinium) dichloride

2.84 g of compound 1 (0.00785 mol) prepared in the preceding step a) are placed in a 100 ml three-necked flask equipped with a condenser, an argon or nitrogen inlet and a magnetic stirring system and 15 ml of ethanol are then added. 1.75 g of 4-picoline (0.0188 mol) are then added and the reaction mixture is refluxed for 10 hours. The reaction progress is monitored by chromatography (HPLC) until the synthesis of compound 2 is complete.

### Step c): Synthesis of Example 4—1,1′-{disulfanediylbis[ethane-2,1-diylimino(4-oxobutane-4,1-diyl)]}bis(4-{(2-[4-(dimethyltetradecylaminium)phenyl]ethenyl}pyridinium) tetrachloride

The aldehyde was synthesized according to the synthetic process described in the following scientific publication: “Novel cationic gemini surfactants as corrosion inhibitors for carbon steel pipeline”, M. A. Hegazy, M. Abdallah, H. Ahmed; Corrosion Science, Vol. 52, Issue 9, pages 2897-2904 (2010).

### Step c) May be Performed in the Following Manner

6.8 g of aldehyde (0.0161 mol) are placed in the reactor of step b) containing compound 2 synthesized in the preceding step b), followed by addition of 40 ml of ethanol at room temperature. The reaction medium is then cooled to 0° C., followed by addition of a solution, prepared beforehand, of pyrrolidine acetate (by adding dropwise, at 0° C., 3.9 g of acetic acid to a solution of 3.3 g of pyrrolidine in 10 ml of ethanol). Once the addition is complete, the reaction medium is maintained at 5° C. for 1 hour and is then allowed to return to room temperature and is maintained at this temperature for 24 hours. The reaction progress is monitored by thin-layer chromatography (TLC). 50 ml of ethanol and then 250 ml of isopropanol are then added to the reaction medium with stirring at 5° C.

The precipitate formed is then filtered off and purified on a column of reverse-phase silica using a polar solvent in the presence of hydrochloric acid.

By following the same process, and just changing for an aldehyde bearing an amino group followed by alkylation in the final step, the following compounds are obtained:

### Example 5: 1,1′-{disulfanediylbis[ethane-2,1-diylimino(4-oxobutane-4,1-diyl)]}bis{4-[(2-(4-{[3-(dimethyl) 3-(hexadecyl)aminium)propyl]amino}phenyl)ethenyl]pyridinium}tetrachloride

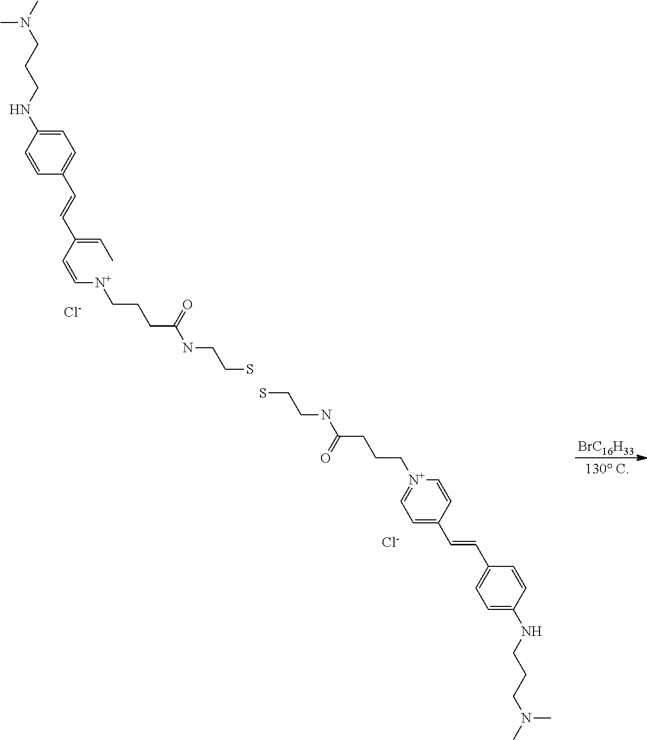 [SMILES: C/C=C(\C=C/[NH2+]CCCC(=O)NCCS)/C=C/c1ccc(NCCCN(C)C)cc1 ; [Cl-] ; CN(C)CCCNc1ccc(/C=C/c2cc[n+](CCCC(=O)NCCS)cc2)cc1 ; [Cl-] ; "BrC16H33"]

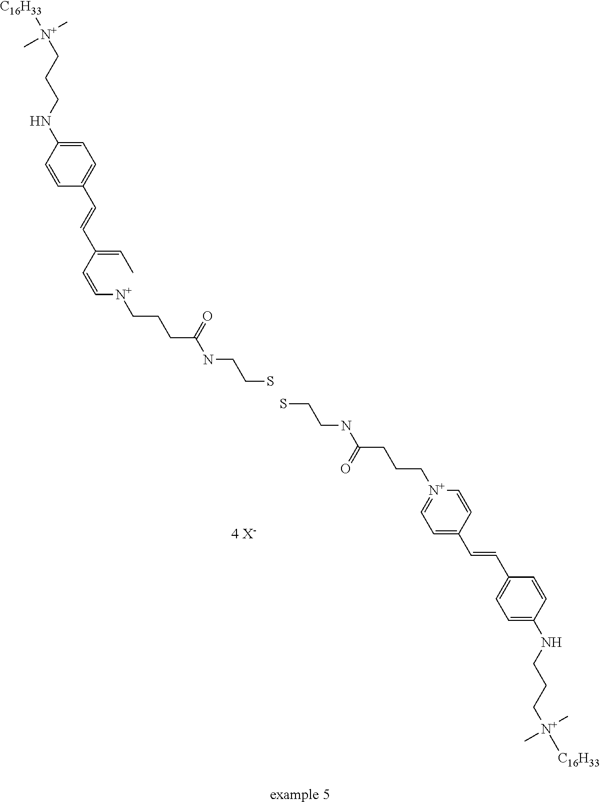

With X⁻, identical or different, which represents Cl⁻ or Br⁻ or a mixture of Cl⁻ and Br⁻.

### Example 6: 1,1′-{disulfanediylbis[ethane-2,1-diylimino(4-oxobutane-4,1-diyl)]}bis{4-[(2-(4-{[3-(dimethyl)-3-(octadecyl)amino)propyl]aminium}phenyl)ethenyl]pyridinium}tetrachloride

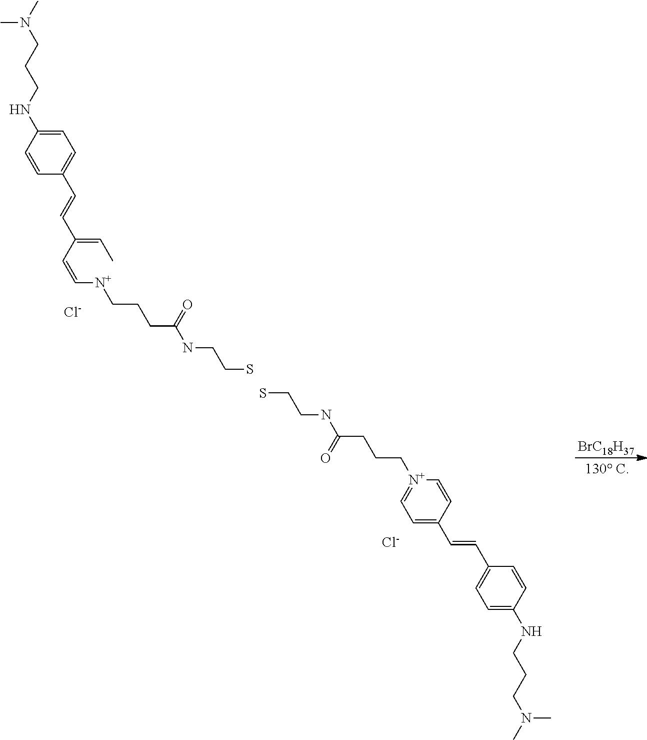 [SMILES: C/C=C(\C=C/[NH2+]CCCC(=O)NCCS)/C=C/c1ccc(NCCCN(C)C)cc1 ; [Cl-] ; CN(C)CCCNc1ccc(/C=C/c2cc[n+](CCCC(=O)NCCS)cc2)cc1 ; [Cl-] ; "BrC18H37"]

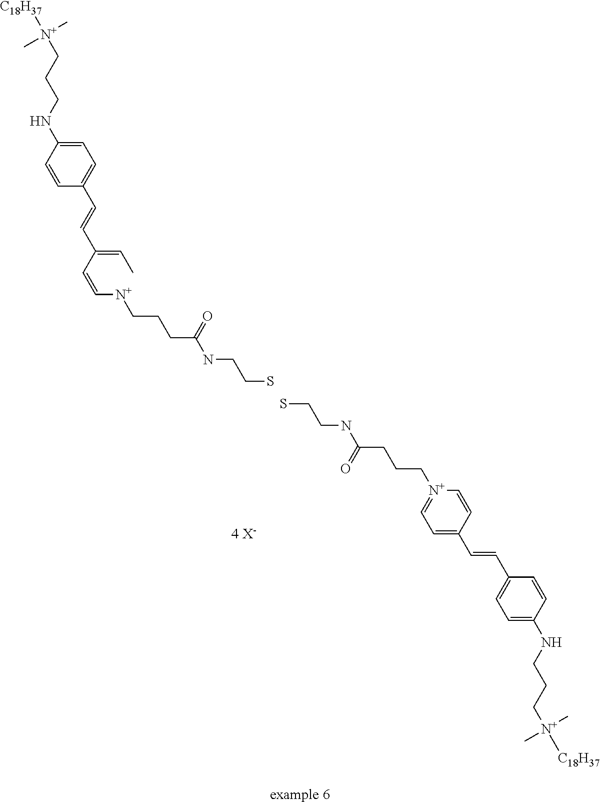

With X⁻, identical or different, which represents Cl⁻ or Br⁻ or a mixture of Cl⁻ and Br⁻.

### Dye Compositions:

Dye compositions at 0.5% by mass were prepared as follows:

0.05 gram (g) of dye (A) (Example 1, 2 or 3) or (B) (Example 4, 5 or 6) is placed in a flask and 10 g of a solvent mixture are added, this mixture being constituted of:
benzyl alcohol (52%)—95% ethanol (33%)—PG 400 polyethylene glycol 400 (2.6%)—water (12.4%) as mass percentages.

The tests are performed on 1 g of locks of grey hair containing 90% white hairs (90NW) and then on 2.7 g of various types of sensitized (bleached) locks.

The dye composition is then applied to the keratin fibres at room temperature, with a leave-on time of 30 minutes.

The three dyes of formula (A) below:

Example 1: 3,3′-{disulfanediylbis[ethane-2,1-diyl(1,3-dioxo-1H-benzo[de]isoquinoline-2,6(3H)-diyl)imino]}bis(N,N-dimethyl-N-tetradecylpropan-1-aminium) dibromide,
Example 2: 3,3′-{disulfanediylbis[ethane-2,1-diyl(1,3-dioxo-1H-benzo[de]isoquinoline-2,6(3H)-diyl)imino]}bis(N,N-dimethyl-N-hexadecylpropan-1-aminium) dibromide,
Example 3: 3,3′-{disulfanediylbis[ethane-2,1-diyl(1,3-dioxo-1H-benzo[de]isoquinoline-2,6(3H)-diyl)imino]}bis(N,N-dimethyl-N-octadecylpropan-1-aminium) dibromide, are evaluated relative to a comparative dye not comprising any fatty chain (A′):

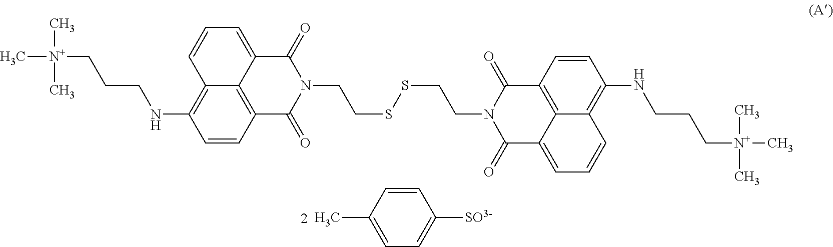

### Results in the L*a*b*system

The colour of the locks was evaluated in the L*a*b* system, using a Minolta® CM 3600D spectrocolorimeter, (Illuminant D65).

In this L*a*b* system, L* represents the lightness, a* indicates the green/red colour axis and b* indicates the blue/yellow colour axis. The higher the value of L, the lighter or less intense the colour. Conversely, the lower the value of L, the darker or more intense the colour. The higher the value of a*, the redder the shade, and the higher the value of b*, the yellower the shade.

The selectivity corresponds to the variation in colouring between the locks of natural hair containing 90% white (90NW) and then dyed and the permanent-waved white (AS20) and then dyed hairs, which is measured by (ΔE) according to the following equation:

ΔE=\sqrt{(L*−Lₒ*)²+(a*−a₀*)²+(b*−b₀*)²}

In this equation, L*, a* and b* represent the values measured after dyeing of the permanent-waved hair AS20, and L₀*, a₀* and b₀* represent the values measured after dyeing of the 90NW hair.

The smaller the selectivity value, the better the homogeneity of the colour between the root and the end of the hairs.

The chromaticity is calculated according to the following formula:

C*=\sqrt{(a*)²+(b*)²}

The higher the value of the chromaticity C*, the more chromatic the colour of the treated keratin fibres.

### Treatment

The locks are treated according to four procedures:
a placebo protocol without straightening
a protocol with straightening using a standard iron
a protocol with straightening using a steampod: steam iron.

Two locks are prepared for each protocol: one is kept at 1 shampoo wash and the other is subjected to persistence at 10 shampoo washes. Thus, eight locks are treated for each dye chosen from Examples 1, 2, 3 or (A′).

Each dye chosen from Examples 1, 2, 3 and (A′) is dissolved at 0.5% by mass in the solvent mixture described previously: it is observed that the solution of Example 1 is slightly darker than those of Examples 2 or 3.

The solutions are applied to 90NW or AS20 locks according to the following protocol:
wet the 1 g lock by treating five times with water at 37° C.; and then
place it in a glass spout;
next, add 2 g of dye solution at 0.5% by mass and then apply to the lock for 20 minutes at room temperature (RT);
wring the lock dry;

Perform Brazilian straightening according to the following protocol:
comb the lock;
blow-dry five times on setting 2 of the hair dryer with a medium-diameter round brush;
optionally, depending on the protocol, treat 10 times with a straightening iron using a comb (or steampod with or without steam);
wash with a mild shampoo; and
dry flat.

The locks treated with Examples 1 to 3 have a more pronounced yellow colour than that of (A′), irrespective of the type of lock treated.

### Study of the Dye of Example 2 on Various Types of Hair

The locks of hair were treated with the following compounds:

The tests are performed on two types of hair:
natural Caucasian hair containing 90% natural white hairs (90NW);
medium bleached Caucasian hair of alkaline solubility 20 (AS20).

### Visual Observation

The visual observation shows that after treatment on the 90NW or AS hair, all the locks have a chromatic yellow colouring, which does not vary significantly after 5 and 10 shampoo washes. This is corroborated by the colorimetric measurements.

### Comparison of the Intensity on Natural White Hairs:

| Process according | | Process with | |
|---|---|---|---|
| to the invention | L* | comparative | L* |
| 5% Example 2 + | 43.97 | 5% Example (A' ) + | 54.44 |
| blow-drying + iron | | blow-drying + iron | |
| 210° C. + 1 sh. wash | | 210° C. + 1 sh. wash | |
| 5% Example 2 + | 55.62 | 5% Example (A') + | 59.9 |
| blow-drying + iron | | blow-drying + iron | |
| 210° C. + 5 sh. washes | | 210° C. + 5 sh. washes | |
| 5% Example 2 + | 55.67 | 5% Example (A') + | 58.53 |
| steampod + | | steampod + | |
| 1 sh. wash | | 1 sh. wash | |
| 5% Example 2 + | 53.22 | 5% Example (A') + | 61.45 |
| steampod + | | steampod + | |
| 10 sh. washes | | 10 sh. washes | |

### Comparison of the Intensity on AS20 Hair:

| Process according | | Process with | |
|---|---|---|---|
| to the invention | L* | comparative | L* |
| 5% Example 2 + | 56.59 | 5% Example (A') + | 60.04 |
| steampod + 1 sh. wash | | steampod + 1 sh. wash | |
| 5% Example 2 + | 57.88 | 5% Example (A') + | 59.09 |
| steampod + 10 sh. washes | | steampod + 10 sh. washes | |

### Comparison of the Chromaticity on AS20 Hair:

| Process | | | | | | | |
|---|---|---|---|---|---|---|---|
| according to the | | | | Process with | | | |
| invention | a* | b* | C* | comparative | a* | b* | C* |
| 5% Example 2 + | 6.94 | 40.73 | 41.31 | 5% Example (A′) + | 3.46 | 39.65 | 39.80 |
| room temperature | | | | RT + 1 sh. wash | | | |
| (RT) + 1 sh. wash | | | | | | | |
| 5% Example 2 + | 6.44 | 37.71 | 38.25 | 5% Example (A′) + | 3.95 | 36.59 | 36.80 |
| RT + 5 sh. washes | | | | RT + 5 sh. washes | | | |

### Comparison of the Selectivity at 10 Shampoo Washes Between AS20 Bleached Hair and 90NW Natural Hair:

| Process according | | Process with | |
|---|---|---|---|
| to the invention | ΔE | comparative | ΔE |
| 5% Example 2 | 11.49 | 5% Example | 15.4 |
| at RT + | | (A') at RT + | |
| 10 sh. washes | | 10 sh. washes | |

It is seen that the aliphatic chain borne by the dye of the invention makes it possible to significantly reduce the selectivity of the colouring between the root and the end, even after 10 shampoo washes, while at the same time improving the intensity of the colouring and the chromaticity.

### Persistence Study—Colorimetric Measurements of the Colour Build-Up ΔE′

One lock was treated for each dye, and the colour was then measured by spectrocolorimetry after 5 and 10 shampoo washes. The colour of a 90NW lock without treatment was also measured so as to have a reference. The colour differences were then calculated relative to the control lock without treatment.

The variation in colouring between the locks of dyed and washed hair and the undyed hair is measured by (ΔE′) according to the following equation:

ΔE=\sqrt{(L*−Lₒ*)²+(a*−a₀*)²+(b*−b₀*)²}

In this equation, L*, a* and b* represent the values measured after dyeing and shampooing, and L₀*, a₀* and b₀* represent the values measured before dyeing.

Results of the spectrocolorimetric measurements for the various Examples 1 (C14), 2 (C16) and 3 (C18) relative to the control (lock without treatment):

First, it is seen that the build-up of the colour is very good for Example 1, 2 or 3 of the invention. The decrease in colour is virtually non-existent after five shampoo washes and particularly small after 10 shampoo washes, which demonstrates the efficient colour persistence on shampooing of the hair with the dyes of the invention.

Specifically, after 10 shampoo washes, Examples 1 to 3 of the invention clearly show a coating effect of the treated lock in comparison with the control lock. There is virtually no difference between the three examples of the invention.

### Durability Study

In order to study the durability of the care effect that may be afforded by the dyes of the invention, tests are performed on various types of hair of different sensitivity and curliness. Thus, bleached type II locks were treated with Example 2 according to the protocols with straightening using an iron and using a steampod, and were then subjected to shampooing so as to evaluate the long-term persistence of the care active agent. From the visual and L*a*b spectrocolorimetry results, it is seen that the colouring is very persistent when the keratin fibres are treated with the dye of Example 2 according to the invention in the presence of an iron (standard or steam iron).

It is also seen that the dyes of the invention (Example 2) make it possible to obtain keratin fibre colourings with very good care persistence afforded to degraded keratin fibres (AS2).

### Sensory Evaluation

### Evaluation Score:

| Effects | Process | Process |
|---|---|---|
| evaluated on | using a | using a steam |
| hair after | straightening | iron of steampod |
| treatment with | iron + | type + |
| example 2 | 40 sh. washes | 40 sh. Washes |
| Softness | 4 | 4 |
| Coating | 3 | 4 |
| Persistence | 4 | 4 |
| Sheen | 3 | 3 |
| Manageability | 4 | 4 |
| Disentangling | 4 | 4 |

The results of the sensory evaluation (evaluated by a score ranging from 1 to 4 with 1 being the poorest score and 4 the best) corroborate the visual observations and the spectroscopic colour measurements: the care effect is persistent even after 40 shampoo washes.

It is seen from this evaluation that the process which uses the step with a steam iron of steampod type is better than the process using a standard straightening iron.

1.-20. (canceled)

21. A process for dyeing keratin materials, comprising applying to the keratin materials at least one cationic, anionic, neutral, or zwitterionic fluorescent direct dye bearing a disulfide function or a thiol or protected-thiol function chosen from compounds of formula (I):
A-(X)ₚ—Cₛₐₜ—S—U (I),
salts thereof with organic or mineral acids or bases, optical or geometric isomers thereof, tautomers thereof, or solvates thereof;
wherein in formula (I):
U represents a radical chosen from: a) —S—C′ₛₐₜ—(X′)_{p′}-A′; and b) —Y;
A and A′, which may be identical or different, represent a cationic, anionic, nonionic or zwitterionic fluorescent chromophore, it being understood that at least one of the two chromophores bears at least one linear or branched, saturated or unsaturated C₁₀-C₃₀ aliphatic chain;
Y represents i) a hydrogen atom; or ii) a thiol-function protecting group;
X and X′, which may be identical or different, represent a linear or branched, saturated or unsaturated divalent C₁-C₃₀ hydrocarbon-based chain, optionally interrupted and/or optionally terminated at one or both of its ends with one or more divalent groups or combinations thereof chosen from:
—N(R)—, —N⁺(R)(R^{o})—, —O—, —C(O)—, —S(O)— and —S(O)₂—, with R and R^{o}, which may be identical or different, chosen from a hydrogen, a C₁-C₄ alkyl, hydroxyalkyl, or aminoalkyl radical;
an aromatic or non-aromatic, saturated or unsaturated, fused or non-fused (hetero)cyclic radical optionally comprising one or more identical or different, optionally substituted heteroatoms;
p and p′, which may be identical or different, are equal to 0 or 1;
Cₛₐₜ and C′ₛₐₜ, which may be identical or different, represent an optionally cyclic, optionally substituted, linear or branched C₁-C₁₈ alkylene chain;
wherein the electrical neutrality of the compounds of formula (I) may be ensured by one or more anionic or, respectively, cationic counterions depending on whether the dye is cationic or, respectively, anionic.

22. The process according to claim 21, wherein the dye(s) of formula (I) are disulfide dyes with U representing a radical a) —S—C′ₛₐₜ—(X′)_{p′}-A′; wherein formula (I) has the following formula:
A-(X)ₚ—Cₛₐₜ—S—S—C′ₛₐₜ—(X′)_{p′}-A′ with A=A′,X=X′,p=p′,Cₛₐₜ=C′ₛₐₜ.

23. The process according to claim 21, wherein the at least one dye of formula (I) is a dye bearing a thiol or protected-thiol function, wherein U represents the radical b) Y, wherein Y represents a hydrogen atom.

24. The process according to claim 21, wherein the at least one dye of formula (I) is a dye with Cₛₐₜ and C′ₛₐₜ, which may be identical or different, representing a chain —(CH₂)ₖ— with k being an integer between 1 and 8 inclusive.

25. The process according to claim 21, wherein the at least one dye of formula (I) is a dye which, when p and p′ is equal to 1, X and X′, which may be identical or different, represent the following sequence:
-(T)ₜ-(Z)_{z}-(T′)_{t′}-
said sequence being linked in formula (I) symmetrically as follows:
—Cₛₐₜ-(T)ₜ-(Z)_{z}-(T′)_{t′}-A or —C′ₛₐₜ-(T)ₜ-(Z)_{z}-(T′)_{t′}-A′;
wherein:
T and T′, which may be identical or different, represent one or more radicals or combinations thereof chosen from: —O—; —S—; —N(R)—; —N⁺(R)(R^{o})—; —S(O)—; —S(O)₂—; —C(O)—; with R, R^{o}, which may be identical or different, representing a hydrogen atom, a C₁-C₄ alkyl radical, C₁-C₄ hydroxyalkyl radical, or an aryl(C₁-C₄)alkyl radical; and a cationic or non-cationic heterocycloalkyl or heteroaryl radical;
t and t′, which may be identical or different, are equal to 0 or 1;
Z represents:
—(CH₂)ₘ— with m being an integer between 1 and 8;
—(CH₂CH₂O)_{q}— or —(OCH₂CH₂)_{q}— wherein q is an integer between 1 and 5 inclusive;
an aryl, alkylaryl, or arylalkyl radical in which the alkyl radical is C₁-C₄, being optionally substituted with at least one group SO₃M with M representing a hydrogen atom, an alkali metal, or an ammonium group substituted with one or more identical or different, linear or branched C₁-C₈ alkyl radicals optionally bearing at least one hydroxyl;
z is 0 or 1.

26. The process according to claim 21, wherein the at least one dye of formula (I) comprises a cationic, anionic, neutral, or zwitterionic fluorescent chromophore, A and/or A′, which may be identical or different, derived from dyes of the following types: acridines; acridones; anthranthrones; anthrapyrimidines; anthraquinones; azines; (poly)azos, hydrazono or hydrazones; benzanthrones; benzimidazoles; benzimidazolones; benzindoles; benzoxazoles; benzopyrans; benzothiazoles; benzoquinones; bisazines; bis-isoindolines; carboxanilides; coumarins; cyanines; (poly)methines; diazines; diketopyrrolopyrroles; dioxazines; diphenylamines; diphenylmethanes; dithiazines; flavonoids; fluorindines; formazans; indamines; indanthrones; indigoids; pseudo-indigoids; indophenols; indoanilines; isoindolines; isoindolinones; isoviolanthrones; naphthalimides; naphthanilides; naphtholactams; naphthoquinones; nitro aromatics; oxadiazoles; oxazines; perilones; perinones; perylenes; phenazines; phenoxazine; phenothiazines; phthalocyanine; polyenes/carotenoids; porphyrins; pyranthrones; pyrazolanthrones; pyrazolones; pyrimidinoanthrones; pyronines; quinacridones; quinolines; quinophthalones; squaranes; tetrazoliums; thiazines, thioindigo; thiopyronines; triarylmethanes; or xanthenes.

27. The process according to claim 21, wherein the keratin materials have a tone depth of less than or equal to 6 before the application.

28. The process according to claim 21, wherein the at least one dye of formula (I) comprises a cationic fluorescent chromophore A and/or A′ which contains at least one quaternary ammonium radical bearing an aliphatic chain chosen from:
a) the polymethine radicals of formulae (II) and (II′) below: 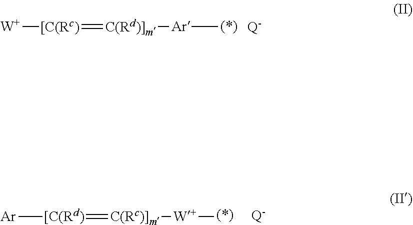
wherein formula (II) or (II′):
W⁺ represents a cationic heterocyclic or heteroaryl group, comprising a quaternary ammonium bearing an aliphatic chain, said heterocyclic or heteroaryl group being optionally substituted with one or more (C₁-C₈)alkyl groups optionally substituted with one or more hydroxyl groups;
W′⁺ represents a cationic heterocyclic or heteroaryl divalent radical comprising a quaternary ammonium, said heterocyclic or heteroaryl group being optionally substituted with one or more (C₁-C₈)alkyl groups optionally substituted with one or more hydroxyl groups; Ar represents an aryl group bearing an exocyclic ammonium charge, with an aliphatic chain, optionally substituted with i) one or more halogen atoms; ii) one or more (C₁-C₈)alkyl groups; iii) one or more hydroxyl groups; iv) one or more (C₁-C₈)alkoxy groups; v) one or more hydroxy(C₁-C₈)alkyl groups, vi) one or more amino or (di)(C₁-C₈)alkylamino groups; vii) with one or more acylamino groups; viii) one or more heterocycloalkyl groups;
Ar′ is a divalent aryl radical chosen from phenyl or naphthyl, optionally substituted with i) one or more halogen atoms; ii) one or more (C₁-C₈)alkyl; iii) one or more hydroxyl groups; iv) one or more (C₁-C₈)alkoxy groups; v) one or more hydroxy(C₁-C₈)alkyl groups, vi) one or more amino or (di)(C₁-C₈)alkylamino groups, vii) with one or more acylamino groups; viii) one or more heterocycloalkyl groups chosen from piperazinyl, piperidyl, or 5- or 6-membered heteroaryl;
m′ represents an integer between 1 and 4 inclusive;
R^{c} and R^{d}, which may be identical or different, represent a hydrogen atom or an optionally substituted (C₁-C₈)alkyl group, or alternatively R^{c} contiguous with W⁺ or W′⁺ and/or R^{d} contiguous with Ar or Ar′ form, with the atoms that bear them, a (hetero)cycloalkyl;
Q⁻, which may be present or absent, is an anionic counterion which serves to ensure the electrical neutrality of the molecule;
(*) represents the part of the chromophore linked to the rest of formula (I);
wherein at least one of the groups W⁺, W′⁺, Ar, or Ar′ bears at least one ammonium group bearing a linear or branched, saturated or unsaturated C₁₀-C₃₀ aliphatic chain;
b) the naphthalimidyl radicals of formula (III) or (III′) below: 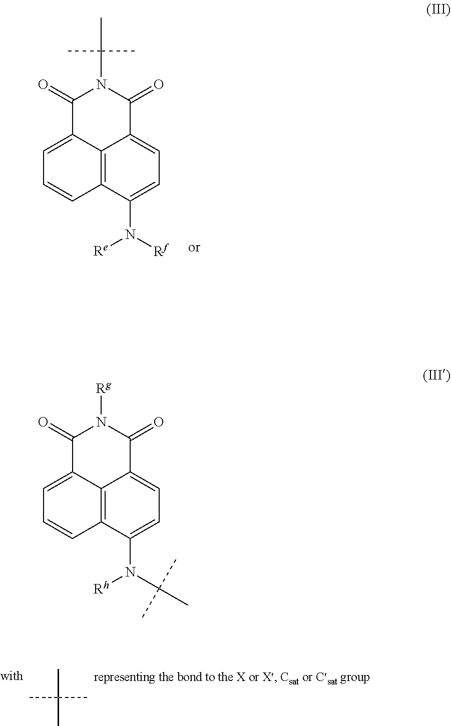
in which formulae (III) and (III′) R^{e}, R^{f}, R^{g} and R^{h}, which may be identical or different, represent a hydrogen atom or a C₁-C₆ alkyl group, of which at least one of the groups R^{e} or R^{f} of the naphthalimidyl (III), or alternatively R^{g} or R^{h} of the naphthalimidyl (IV) is substituted with a trialkylammonium group RₐR_{b}R_{c}N⁺—, An⁻, with Rₐ and R_{b}, which may be identical or different, representing a (C₁-C₃₀)alkyl group and R_{c} representing a C₁₀-C₃₀, and An⁻, which may be present or absent, representing an anionic counterion which ensures the electrical neutrality of the molecule.

29. The process according to claim 21, wherein the at least one dye of formula (I) is a disulfide dye chosen from those of formulae (IV) to (VIII) and thiol or protected-thiol dyes chosen from those of formulae (IV′) to (VIII′) below: 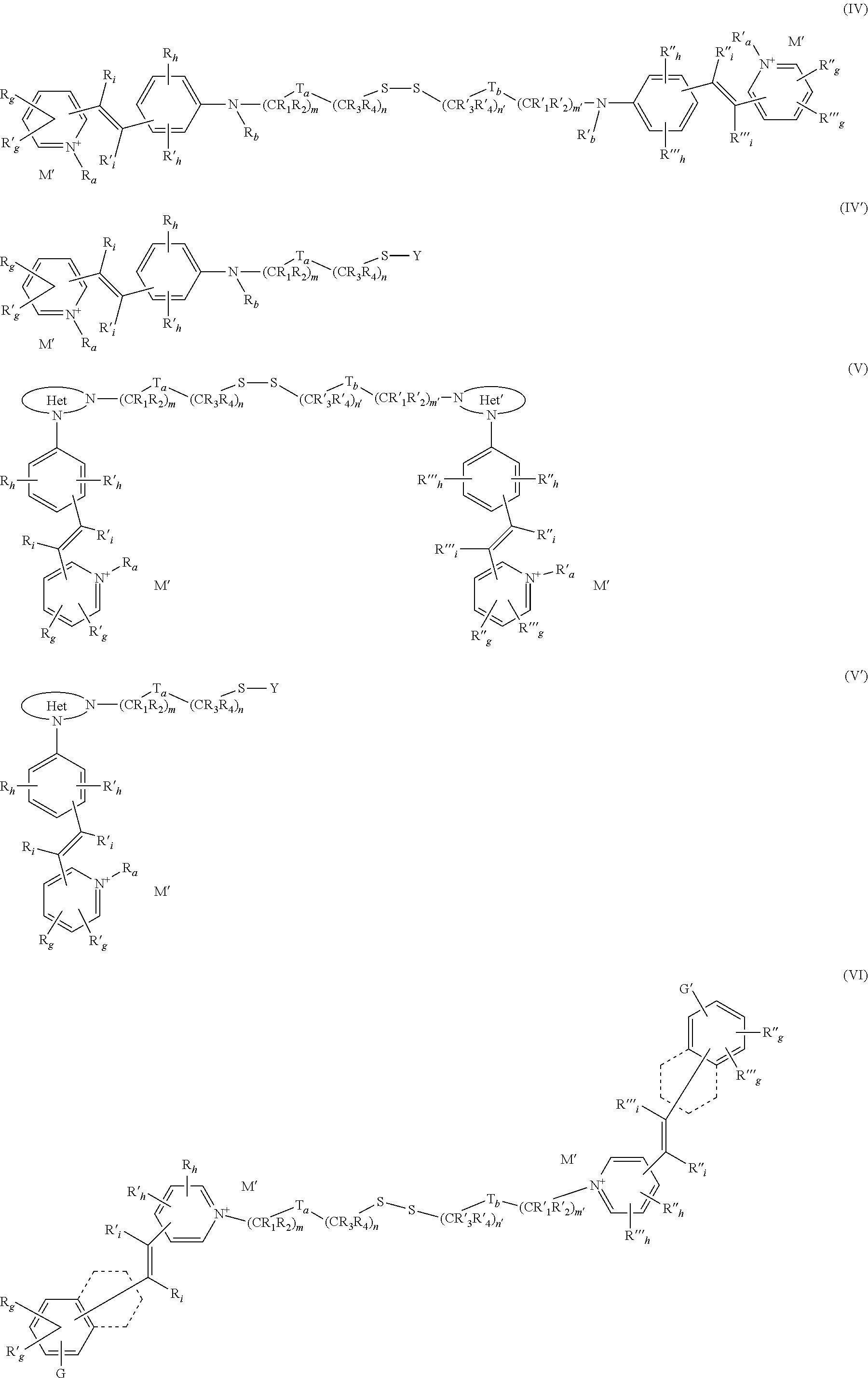 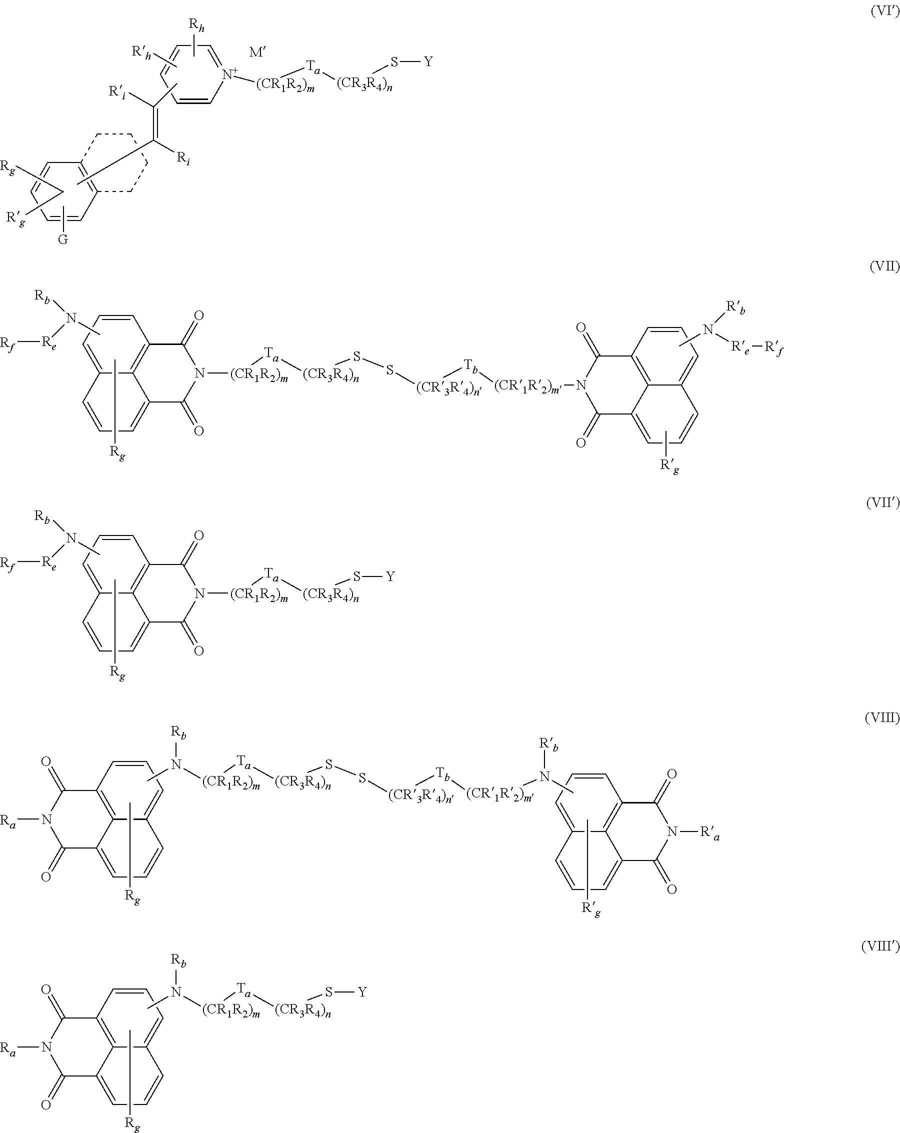
organic or mineral acid salts, optical isomers and geometrical isomers thereof, or solvates thereof;
in which formulae (IV) to (VIII) and (IV′) to (VIII′):
G and G′, which may be identical or different, represent i) a group —NR_{c}R_{d}, —NR′_{c}R′_{d}, ii) C₁-C₆ alkoxy which is optionally substituted; or iii) a group R¹R²R³N⁺—, An⁻ with R¹ representing a linear or branched (C₁₀-C₃₀)alkyl; and R² and R³, which may be identical or different, represent a linear or branched (C₁-C₃₀) alkyl group, and An⁻, which may be present or absent, represents an organic or mineral anionic counterion;
Rₐ and R′ₐ, which may be identical or different, represent an aryl(C₁-C₄)alkyl group or a C₁-C₆ alkyl group optionally substituted with a hydroxyl or amino, C₁-C₄ alkylamino or C₁-C₄ dialkylamino group, said alkyl radicals optionally forming, with the nitrogen atom that bears them, a 5- to 7-membered heterocycle, optionally comprising another nitrogen or non-nitrogen heteroatom;
R_{b} and R′_{b}, which may be identical or different, represent a hydrogen atom, an aryl(C₁-C₄)alkyl group, or a C₁-C₆ alkyl group that is optionally substituted;
R_{c}, R′_{c}, R_{d} and R′_{d}, which may be identical or different, represent a hydrogen atom, an aryl(C₁-C₄)alkyl, C₁-C₆ alkoxy group, or a C₁-C₆ alkyl group that is optionally substituted;
or alternatively two adjacent radicals R_{c} and R_{d}, R′_{c} and R′_{d} borne by the same nitrogen atom together form a heterocyclic or heteroaryl group;
Rₑ and R′ₑ, which may be identical or different, represent a linear or branched, optionally unsaturated, divalent C₁-C₆ alkylenyl hydrocarbon-based chain;
R_{f} and R′_{f}, which may be identical or different, represent a quaternary ammonium group R¹R²R³N⁺—;
R_{g}, R′_{g}, R″_{g}, R′″_{g}, Rₕ, R′ₕ, R″ₕ and R′″ₕ, which may be identical or different, represent a hydrogen atom, a halogen atom, an amino, C₁-C₄ alkylamino, C₁-C₄ dialkylamino, cyano, carboxyl, hydroxyl group, trifluoromethyl group, an acylamino, C₁-C₄ alkoxy, (poly)hydroxy(C₂-C₄)alkoxy, alkylcarbonyloxy, alkoxycarbonyl radical, alkylcarbonylamino radical, an acylamino, carbamoyl or alkylsulfonylamino radical, an aminosulfonyl radical, or a C₁-C₁₆ alkyl radical optionally substituted with a group chosen from C₁-C₁₂ alkoxy, hydroxyl, cyano, carboxyl, amino, C₁-C₄ alkylamino, or C₁-C₄ dialkylamino, or alternatively the two alkyl radicals borne by the nitrogen atom of the amino group form a 5- to 7-membered heterocycle optionally comprising another heteroatom identical to or different from that of the nitrogen atom;
or alternatively two groups R_{g} and R′_{g}; R″_{g} and R′″_{g}; Rₕ and R′ₕ; R″ₕ and R′″ₕ borne by two adjacent carbon atoms together form a benzo or indeno ring, a fused heterocycloalkyl or fused heteroaryl group; the benzo, indeno, heterocycloalkyl or heteroaryl ring being optionally substituted with a halogen atom, an amino, C₁-C₄ alkylamino, C₁-C₄ dialkylamino, nitro, cyano, carboxyl, hydroxyl or trifluoromethyl group, an acylamino, C₁-C₄ alkoxy, (poly)hydroxy(C₂-C₄)alkoxy, alkylcarbonyloxy, alkoxycarbonyl or alkylcarbonylamino radical, an acylamino, carbamoyl or alkylsulfonylamino radical, an aminosulfonyl radical, or a C₁-C₁₆ alkyl radical optionally substituted with: a group chosen from C₁-C₁₂ alkoxy, hydroxyl, cyano, carboxyl, amino, C₁-C₄ alkylamino, C₁-C₄ dialkylamino, or alternatively the two alkyl radicals borne by the nitrogen atom of the amino group form a 5- to 7-membered heterocycle optionally comprising another heteroatom identical to or different from that of the nitrogen atom;
or alternatively when G represents —NR_{c}R_{d} and G′ represents —NR′_{c}R′_{d}, two groups R_{c} and R′_{g}; R′_{c} and R″_{g}; R_{d} and R_{g}; R′_{d} and R′″_{g} together form a saturated heteroaryl or heterocycle, optionally substituted with one or more groups C₁-C₆ alkyl;
Rᵢ, R′ᵢ, R″ᵢ and R′″ᵢ, which may be identical or different, represent a hydrogen atom or a C₁-C₄ alkyl group;
R₁, R₂, R₃, R₄, R′₁, R′₂, R′₃ and R′₄, which may be identical or different, represent a hydrogen atom or a C₁-C₄ alkyl, C₁-C₁₂ alkoxy, hydroxyl, cyano, carboxyl, amino, C₁-C₄ alkylamino or C₁-C₄ dialkylamino group, said alkyl radicals possibly forming with the nitrogen atom that bears them a 5- to 7-membered heterocycle optionally comprising another nitrogen or non-nitrogen heteroatom;
Tₐ and T_{b}, which may be identical or different, represent i) a σ covalent bond, ii) or one or more radicals or combinations thereof chosen from —SO₂—, —O—, —S—,
—N(R)—, —N⁺(R)(R^{o})— and —CO—, with R and R^{o}, which may be identical or different, representing a hydrogen atom, a C₁-C₄ alkyl radical or a C₁-C₄ hydroxyalkyl radical, or an aryl(C₁-C₄)alkyl radical; 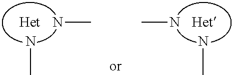
which may be identical or different, represent a heterocyclic group, which is optionally substituted with a quaternary ammonium group R¹R²R³N⁺—; 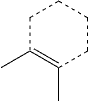
represents an aryl or heteroaryl group fused to the imidazolium or phenyl ring; or alternatively is absent from the imidazolium or phenyl ring;
m, m′, n and n′, which may be identical or different, represent an integer between 0 and 6 inclusive, with m+n and m′+n′, which may be identical or different, representing an integer between 1 and 10 inclusive;
Y represents a hydrogen atom or a protecting group chosen from:
(C₁-C₄)alkylcarbonyl, for instance methylcarbonyl or ethylcarbonyl;
arylcarbonyl, for instance phenylcarbonyl;
(C₁-C₄)alkoxycarbonyl;
aryloxycarbonyl;
aryl(C₁-C₄)alkoxycarbonyl;
(di)(C₁-C₄)(alkyl)aminocarbonyl;
(C₁-C₄)(alkyl)arylaminocarbonyl;
optionally substituted aryl;
5- or 6-membered monocyclic heteroaryl;
cationic 5- or 6-membered monocyclic heteroaryl; these groups being optionally substituted with one or more identical or different (C₁-C₄)alkyl groups;
cationic 8- to 11-membered bicyclic heteroaryl; these groups being optionally substituted with one or more identical or different (C₁-C₄)alkyl groups;
cationic heterocycle having the following formula: 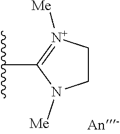 [SMILES: *C1=[N+](C)CCN1C]
—C(NH₂)═N⁺H₂; An′″⁻;
—C(NH₂)═NH;
SO₃⁻, M⁺ with M⁺ representing an alkali metal; and
M′ represents an anionic counterion, derived from a salt of an organic or mineral acid, or from an organic or mineral base that ensures the electrical neutrality of the dye molecule;
wherein at least one of the groups G, G′, Rₐ, R′ₐ, R_{c}, R′_{c}, R_{d}, R′_{d}, R_{f} or R′_{f} bears at least one group R¹R²R³N⁺—.

30. The process according to claim 21, wherein the at least one dye of formula (I) is chosen from those of formulae (IX) to (X′): 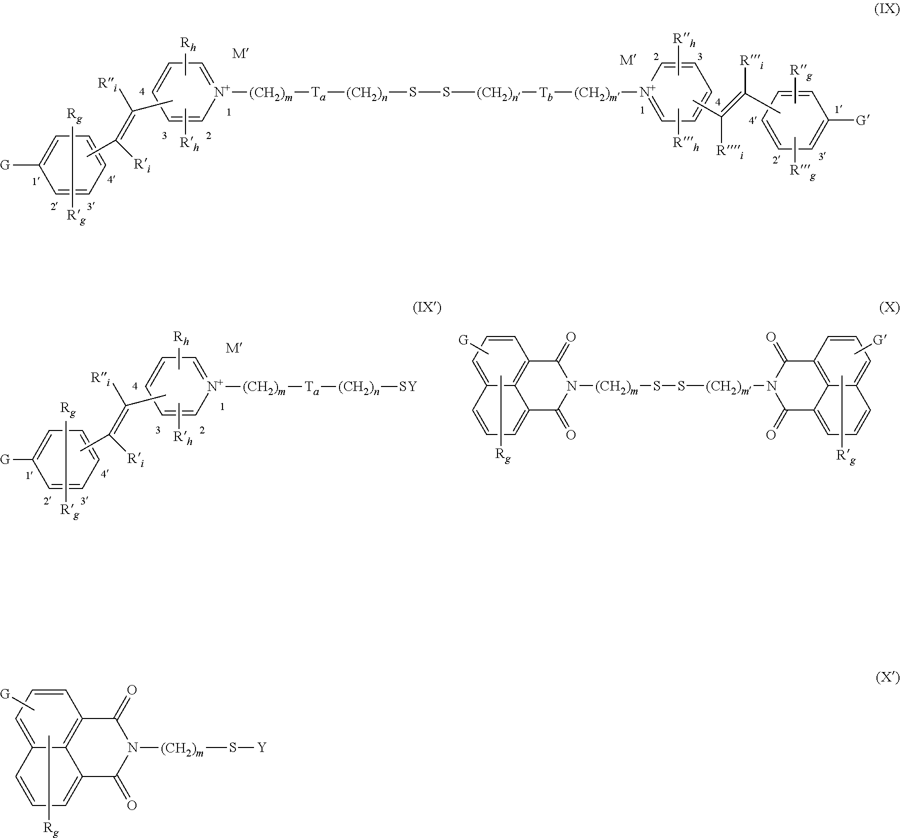
organic or mineral acid salts, optical isomers or geometrical isomers thereof, or the solvates;
wherein formulae (IX) to (X′):
G and G′, which may be identical or different, represent i) a (di)(C₁-C₆)alkylamine group with at least one of the alkyl groups substituted with an ammonium group R¹R²R³N⁺— or ii) an ammonium group R¹R²R³N⁺—;
R_{g}, R′_{g}, R″_{g}, R′″_{g}, Rₕ, R′ₕ, R″ₕ and R′″ₕ, which may be identical or different, represent a hydrogen or halogen atom, an amino, (di)(C₁-C₄)alkylamino, cyano, carboxyl, hydroxyl, trifluoromethyl, acylamino, C₁-C₄ alkoxy, C₂-C₄ (poly)hydroxyalkoxy, (C₁-C₄)alkylcarbonyloxy, (C₁-C₄)alkoxycarbonyl, (C₁-C₄)alkylcarbonylamino, acylamino, carbamoyl or (C₁-C₄)alkylsulfonylamino group, an aminosulfonyl radical or a (C₁-C₁₆)alkyl radical optionally substituted with a group chosen from (C₁-C₁₂)alkoxy, hydroxyl, cyano, carboxyl, amino and (di)(C₁-C₄)alkylamino, or alternatively the two alkyl radicals borne by the nitrogen atom of the amino group form a 5- to 7-membered heterocycle optionally comprising another nitrogen or non-nitrogen heteroatom;
R′ᵢ, R″ᵢ, R′″ᵢ and R″″ᵢ, which may be identical or different, represent a hydrogen atom or a (C₁-C₄)alkyl group; in particular R′ᵢ, R″ᵢ, R′″ᵢ, and R″″ᵢ represent a hydrogen atom;
Tₐ and T_{b}, which may be identical or different, represent i) either a σ covalent bond, ii) or one or more radicals or combinations thereof chosen from —O—, —N(R)— and —C(O)—, with R representing a hydrogen atom or a C₁-C₄ alkyl radical
m, m′, n and n′, which may be identical or different, represent an integer between 0 and 6 inclusive, with m+n and m′+n′, which may be identical or different, representing an integer between 1 and 10 inclusive;
wherein the bond between the pyridinium ring and the double bond of the ethylene or styryl group is located in position 2 or 4 of the pyridinium.

31. The process according to claim 21, wherein the at least one dye of formula (I) is chosen from those below: 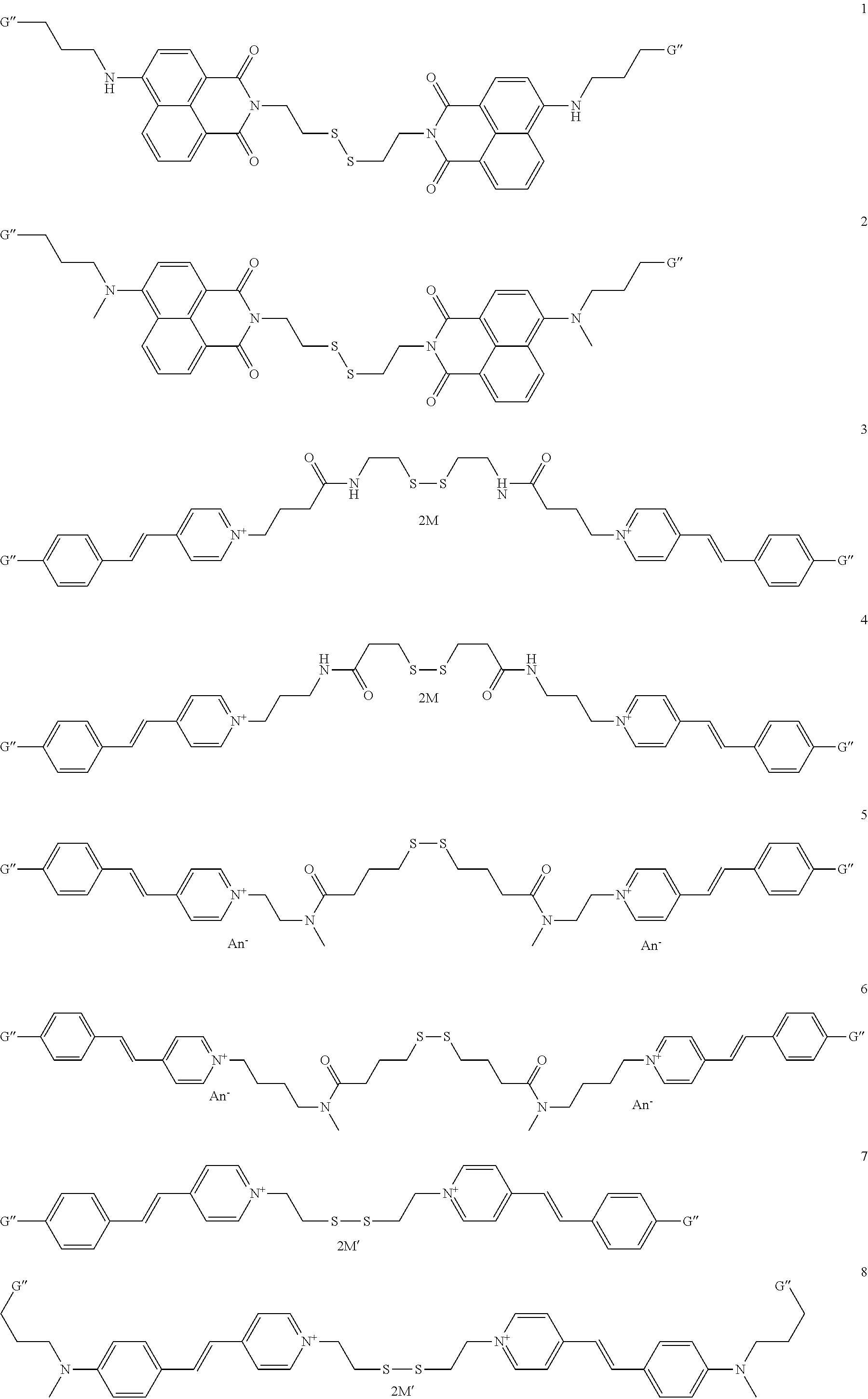 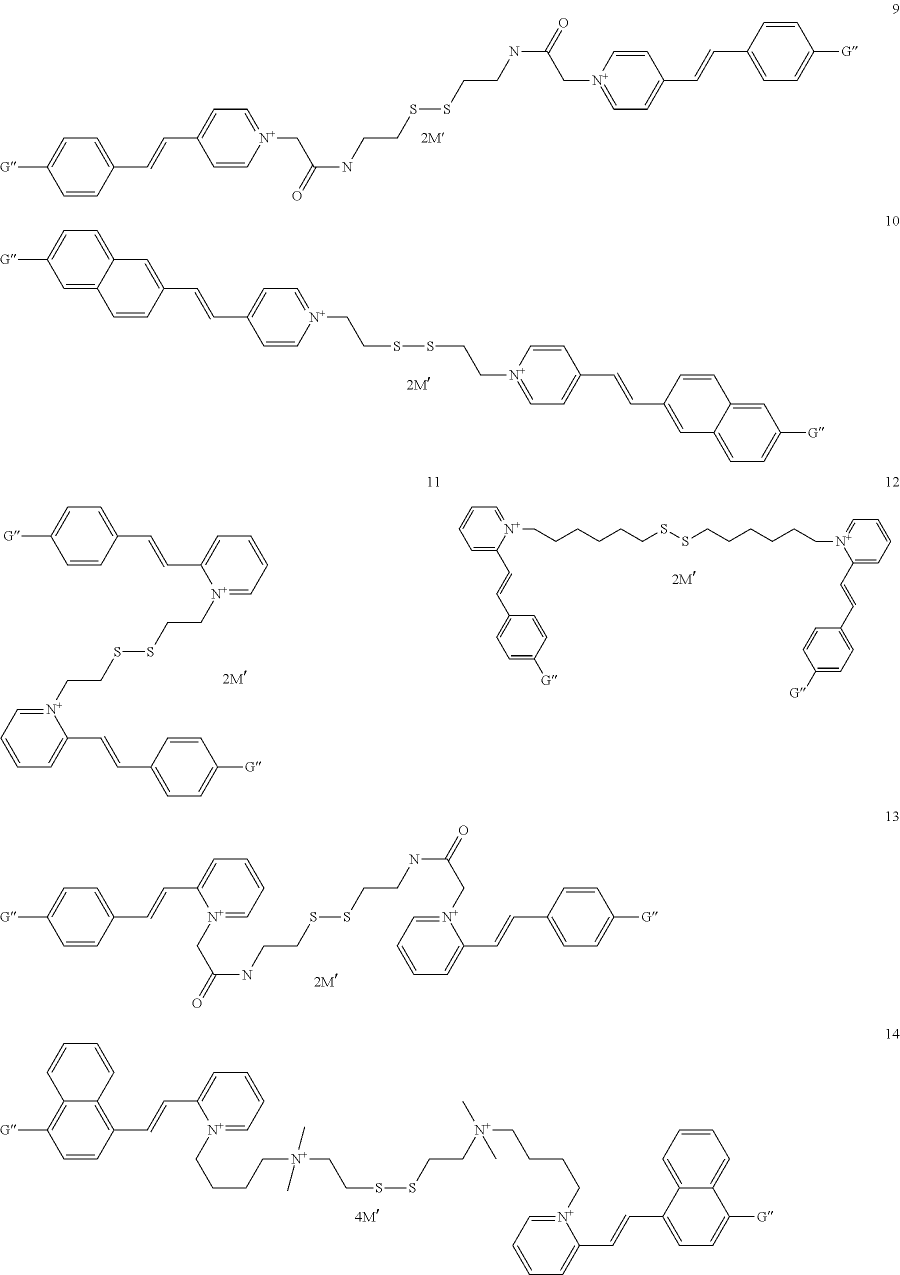 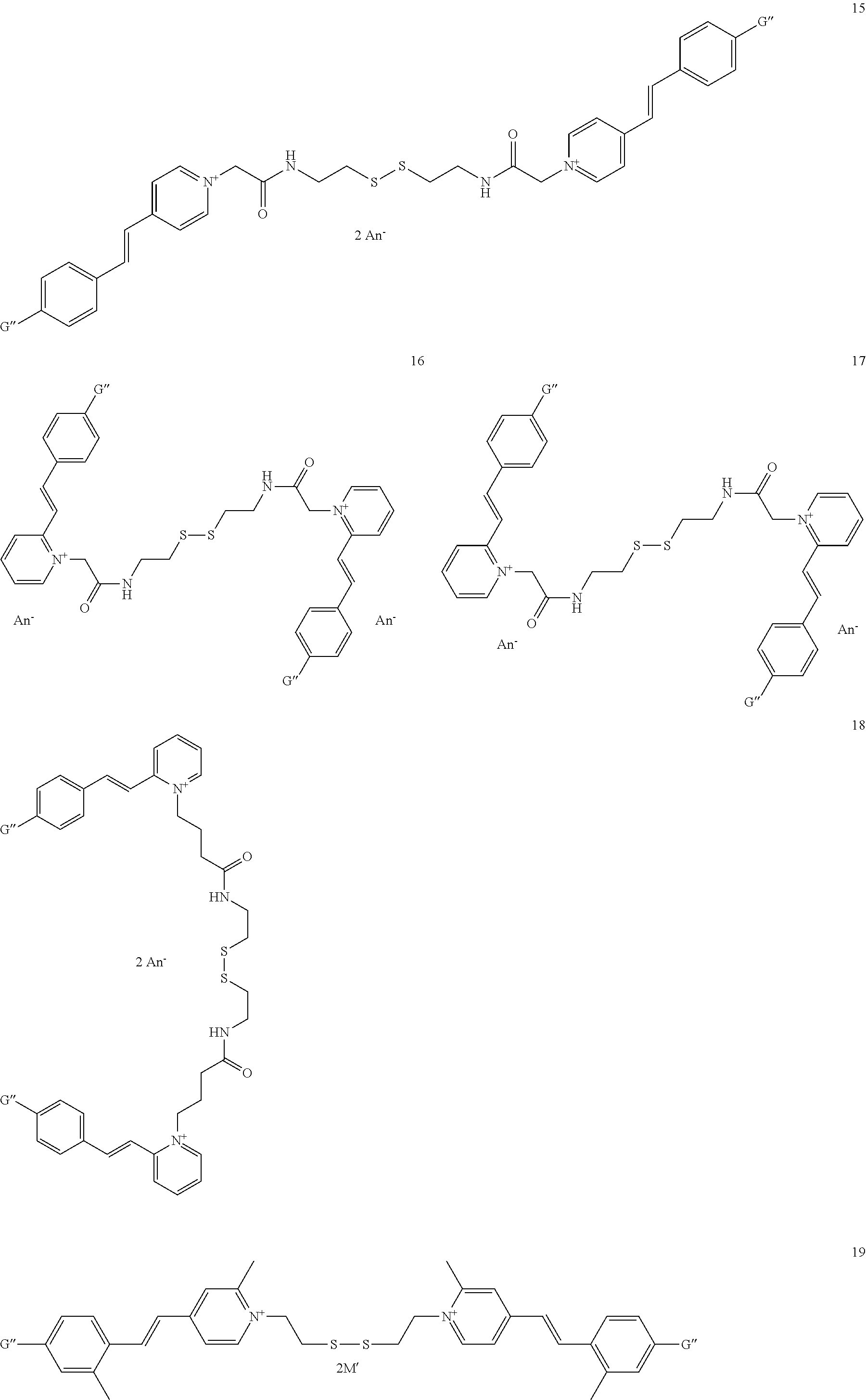 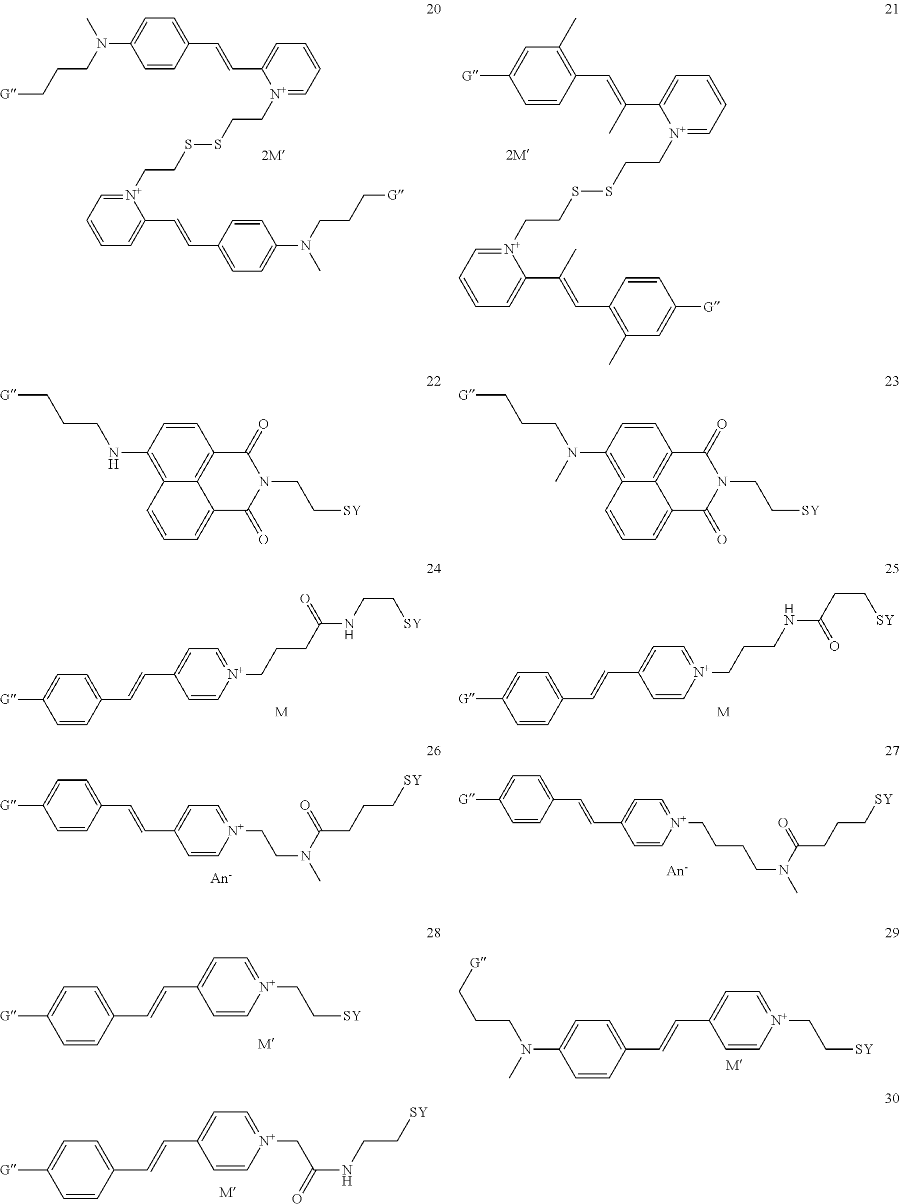
and also organic or mineral acid salts and geometrical isomers thereof, and solvates thereof; with An⁻, M′, which may be identical or different, representing anionic counterions.

32. The process according to claim 21, wherein the at least one dye of formula (I) is chosen from (A) and (B) below: 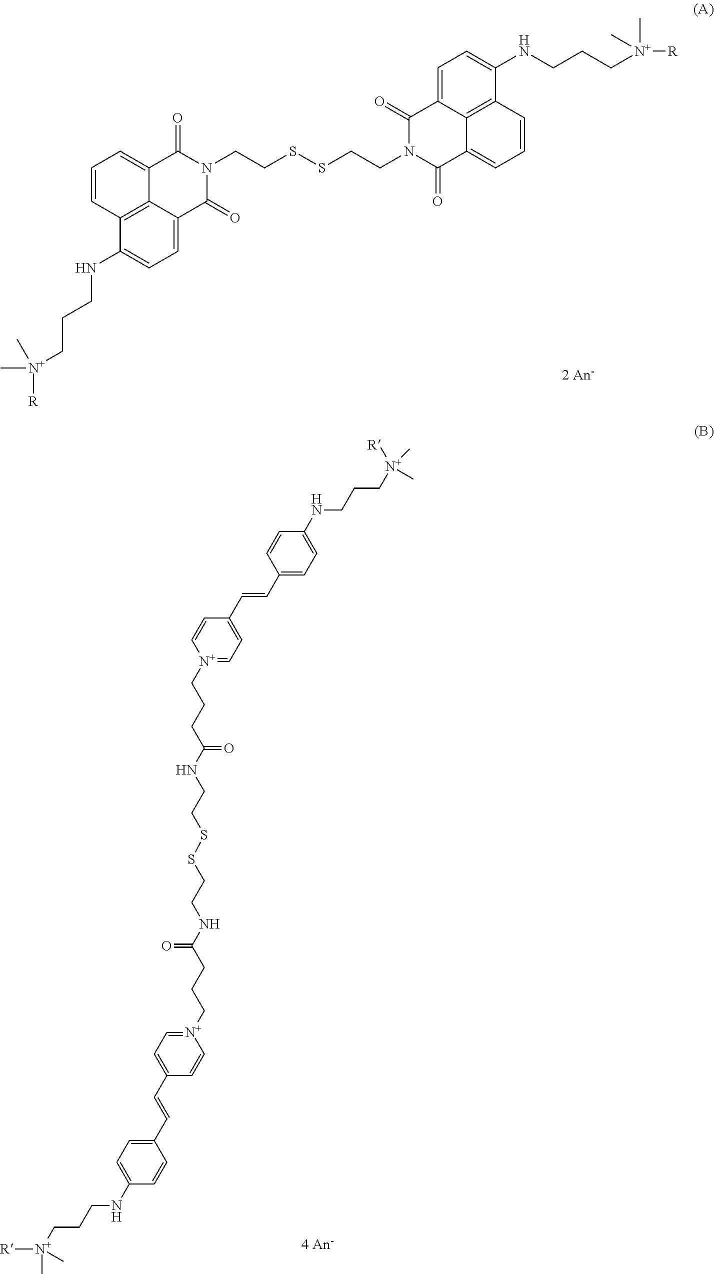
and also organic or mineral acid salts thereof, geometrical isomers thereof, or solvates thereof;
in which formulae (A) and (B):
R, which are identical, represent a linear (C₁₀-C₃₀)alkyl group;
R′, which are identical, represent a linear (C₁₀-C₃₀)alkyl group; and
An⁻, which may be identical or different, is chosen from a halide.

33. The process according to claim 21, wherein the process comprises i) applying to the keratin materials at least one fluorescent dye bearing a disulfide, thiol or protected-thiol function of formula (I) and ii) applying to the keratin materials a heat source chosen from steam or a straightening iron.

34. The process according to claim 33, wherein applying steam to the keratin materials is performed after applying to the keratin materials i) at least one fluorescent direct dye bearing a disulfide function, a thiol, or protected-thiol function.

35. The process according to claim 21, wherein the process does not use a reducing agent.

36. The process according to claim 21, wherein the process does not use a chemical oxidizing agent.

37. The process according to claim 21, wherein the tone depth of the keratin materials is less than or equal to 4.

38. A compound chosen from compounds of formula (I):
A-(X)ₚ—Cₛₐₜ—S—U (I),
salts thereof with organic or mineral acids or bases, optical or geometric isomers thereof, tautomers thereof, or solvates thereof;
wherein in formula (I):
U represents a radical chosen from: a) —S—C′ₛₐₜ—(X′)_{p′}-A′; and b) —Y;
A and A′, which may be identical or different, represent a cationic, anionic, nonionic or zwitterionic fluorescent chromophore, it being understood that at least one of the two chromophores bears at least one linear or branched, saturated or unsaturated C₁₀-C₃₀ aliphatic chain;
Y represents i) a hydrogen atom; or ii) a thiol-function protecting group;
X and X′, which may be identical or different, represent a linear or branched, saturated or unsaturated divalent C₁-C₃₀ hydrocarbon-based chain, optionally interrupted and/or optionally terminated at one or both of its ends with one or more divalent groups or combinations thereof chosen from:
—N(R)—, —N⁺(R)(R^{o})—, —O—, —C(O)—, —S(O)— and —S(O)₂—, with R and R^{o}, which may be identical or different, chosen from a hydrogen, a C₁-C₄ alkyl, hydroxyalkyl, or aminoalkyl radical;
an aromatic or non-aromatic, saturated or unsaturated, fused or non-fused (hetero)cyclic radical optionally comprising one or more identical or different, optionally substituted heteroatoms;
p and p′, which may be identical or different, are equal to 0 or 1;
Cₛₐₜ and C′ₛₐₜ, which may be identical or different, represent an optionally cyclic, optionally substituted, linear or branched C₁-C₁₈ alkylene chain;
wherein the electrical neutrality of the compounds of formula (I) may be ensured by one or more counterions.

39. A composition comprising at least one compound of claim 38.
